(19) United States
(12) Patent Application Publication
(10) Pub. No.: US 2025/0041312 A1
(43) Pub. Date: Feb. 6, 2025

(54) METHODS OF ADMINISTERING A MODULATOR OF HEMOGLOBIN

(71) Applicant: Global Blood Therapeutics, Inc., South San Francisco, CA (US)

(72) Inventors: Aline Barth, Collegeville, PA (US); Ken Duchin, Delray Beach, FL (US); Carla B. Washington, San Francisco, CA (US); Arthur Lo, San Francisco, CA (US)

(73) Assignee: Global Blood Therapeutics, Inc., South San Francisco, CA (US)

(21) Appl. No.: 18/717,181

(22) PCT Filed: Dec. 9, 2022
(86) PCT No.: PCT/US2022/052411

§ 371 Date: Jun. 6, 2024

Related U.S. Application Data
(60) Provisional application No. 63/288,377, filed on Dec. 10, 2021.
(60) Provisional application No. 63/421,524, filed on Nov. 1, 2022.
(60) Provisional application No. 63/429,376, filed on Dec. 1, 2022.

(51) Int. Cl. A61K 31/5377; A61K 31/17; A61P 7/06

(52) U.S. Cl. CPC A61K 31/5377; A61K 31/17; A61P 7/06

(57) ABSTRACT

Provided herein are methods for treating sickle cell disease comprising administering Compound (I), a modulator of hemoglobin, according to certain dosing regimens.

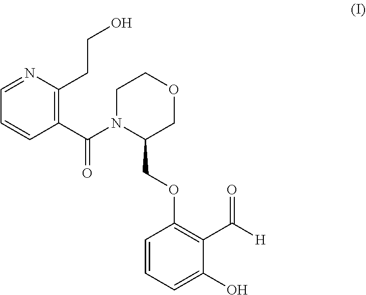 [SMILES: O=Cc1c(O)cccc1OC[C@@H]1COCCN1C(=O)c1cccnc1CCO]

### CROSS-REFERENCE TO RELATED APPLICATIONS

This application claims the benefit under 35 U.S.C. § 119(e) of U.S. Provisional Application No. 63/288,377, filed Dec. 10, 2021, U.S. Provisional Application No. 63/421,524, filed Nov. 1, 2022, and U.S. Provisional Application No. 63/429,376, filed Dec. 1, 2022, each of which is hereby incorporated by reference in its entirety.

### FIELD

The present disclosure provides for methods for treating sickle cell disease comprising administering Compound I, (S)-2-hydroxy-6-((4-(2-(2-hydroxyethyl)nicotinoyl)morpholin-3-yl)methoxy)benzaldehyde, or a pharmaceutically acceptable salt thereof, a modulator of hemoglobin, according to certain dosing regimens.

### BACKGROUND

Sickle cell disease (SCD) is a disorder of the red blood cells, found particularly among those of African and Mediterranean descent. The basis for SCD is found in sickle hemoglobin (HbS), which contains a point mutation relative to the prevalent peptide sequence of hemoglobin A (HbA).

Hemoglobin (Hb) transports oxygen molecules from the lungs to various tissues and organs throughout the body. Hemoglobin binds and releases oxygen through conformational changes. Sickle hemoglobin (HbS) contains a point mutation where glutamic acid is replaced with valine, making HbS susceptible to polymerization under hypoxic conditions to give the HbS containing red blood cells (RBCs) their characteristic sickle shape. The sickled cells are also more rigid than normal RBCs, and their lack of flexibility can lead to blockage of blood vessels.

Compounds, such as Compound I, that modulate hemoglobin and are useful in treating disorders mediated by abnormal Hb (such as HbS) are disclosed in U.S. Pat. No. 10,683,285, the disclosure of which is hereby incorporated by reference in its entirety.

### SUMMARY

The present disclosure provides for methods for treating sickle cell disease in a subject in need thereof, comprising administering to the subject a first dose of Compound I of formula:

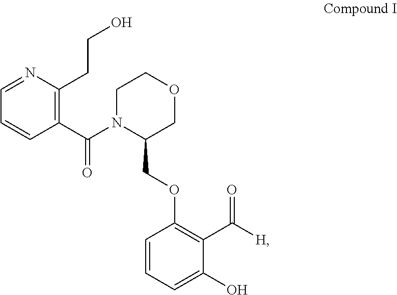

or a pharmaceutically acceptable salt thereof, followed by a second dose of Compound I, or a pharmaceutically acceptable salt thereof.

Some embodiments provide for a method for treating sickle cell disease in a subject in need thereof, comprising administering to the subject a first dose of Compound I, or a pharmaceutically acceptable salt thereof, for one, two, three, four, five, six, or seven days, followed thereafter by a second dose of Compound I, or a pharmaceutically acceptable salt thereof, wherein the administrations of the first dose and the second dose provide about 25% to about 60% hemoglobin occupancy of Compound I.

Some embodiments provide for a method for treating sickle cell disease in a subject in need thereof, comprising administering to the subject a first dose of about 200 mg per day to about 1600 mg per day of Compound I, or a pharmaceutically acceptable salt thereof, for one, two, three, four, five, six, or seven days, followed thereafter by a second dose of about 25 mg per day to about 500 mg per day of Compound I, or a pharmaceutically acceptable salt thereof.

Some embodiments provide for a method for treating sickle cell disease in a subject in need thereof, comprising administering to the subject a first dose of about 200 mg twice daily (BID) of Compound I, or a pharmaceutically acceptable salt thereof, for four days, followed thereafter by a second dose of 100 mg per day of Compound I, or a pharmaceutically acceptable salt thereof.

Some embodiments provide for a method for treating sickle cell disease in a subject in need thereof, comprising administering to the subject a first dose of about 300 mg twice daily (BID) of Compound I, or a pharmaceutically acceptable salt thereof, for four days, followed thereafter by a second dose of 150 mg per day of Compound I, or a pharmaceutically acceptable salt thereof.

Some embodiments provide for a method for treating sickle cell disease in a subject in need thereof, comprising administering to the subject a first dose of about 400 mg twice daily (BID) of Compound I, or a pharmaceutically acceptable salt thereof, for four days, followed thereafter by a second dose of 200 mg per day of Compound I, or a pharmaceutically acceptable salt thereof.

Some embodiments provide for a compound for use in treating sickle cell disease in a subject in need thereof, comprising administering to the subject a first dose of Compound I, or a pharmaceutically acceptable salt thereof, for one, two, three, four, five, six, or seven days, followed thereafter by a second dose of Compound I, or a pharmaceutically acceptable salt thereof, wherein the administrations of the first dose and the second dose provide about 25% to about 60% hemoglobin occupancy by Compound I.

Some embodiments provide for a compound for use in treating sickle cell disease in a subject in need thereof, comprising administering to the subject a first dose of about 200 mg per day to about 1600 mg per day of Compound I, or a pharmaceutically acceptable salt thereof, for one, two, three, four, five, six, or seven days, followed thereafter by a second dose of about 25 mg per day to about 500 mg per day of Compound I, or a pharmaceutically acceptable salt thereof.

Some embodiments provide for a compound for use in treating sickle cell disease in a subject in need thereof, comprising administering to the subject a first dose of about 200 mg twice daily (BID) of Compound I, or a pharmaceutically acceptable salt thereof, for four days, followed thereafter by a second dose of 100 mg per day of Compound I, or a pharmaceutically acceptable salt thereof.

Some embodiments provide for a compound for use in treating sickle cell disease in a subject in need thereof, comprising administering to the subject a first dose of about 300 mg twice daily (BID) of Compound I, or a pharmaceutically acceptable salt thereof, for four days, followed thereafter by a second dose of 150 mg per day of Compound I, or a pharmaceutically acceptable salt thereof.

Some embodiments provide for a compound for use in treating sickle cell disease in a subject in need thereof, comprising administering to the subject a first dose of about 400 mg twice daily (BID) of Compound I, or a pharmaceutically acceptable salt thereof, for four days, followed thereafter by a second dose of 200 mg per day of Compound I, or a pharmaceutically acceptable salt thereof.

### BRIEF DESCRIPTION OF THE DRAWINGS

FIG. 1 shows that single doses of Compound I (top line) exhibit a dose-dependent increase in percent Hb occupancy in healthy volunteers. These Hb occupancies, after single doses of Compound I, exceed those reported in healthy volunteers receiving single doses of voxelotor (bottom line) over a similar range as reported in Br J Clin Pharmacol. 2019; 28(6):1290-1302.

FIG. 2 shows the percent hemoglobin occupancy of Compound I in healthy volunteers after receiving first (loading) daily doses of 300 mg of Compound I for 3 days followed by second (maintenance) daily doses of 15 mg, 25 mg, 50 mg, or 75 mg for 14 days.

FIG. 3 shows a general outline for single dose (SD) of 100 mg and multiple ascending doses (MAD) at second daily dose levels of 50 mg (MAD-1), 100 mg (MAD-2), and 150 mg (MAD-3) in SCD patients as detailed in Example 3.

FIG. 4 shows the change in hemoglobin following both MAD-1 and MAD-2 dosing (i.e. at 8 weeks following these two rounds of treatment) of Compound I in six SCD patients.

FIG. 5 shows the change in reticulocytes at baseline and after both MAD-1 and MAD-2 dosing (i.e. after 8 weeks of treatment) of Compound I in six SCD patients.

FIG. 6 shows the change in absolute reticulocytes at baseline and after both MAD-1 and MAD-2 dosing (i.e. after 8 weeks of treatment) of Compound I in six SCD patients.

FIG. 7 shows the change in lactate dehydrogenase (LDH) at baseline and after both MAD-1 and MAD-2 dosing (i.e. after 8 weeks of treatment) of Compound I in six SCD patients.

FIG. 8 shows the change in indirect bilirubin at baseline and after both MAD-1 and MAD-2 dosing (i.e. after 8 weeks of treatment) of Compound I in six SCD patients.

FIG. 9 shows Oxygenscan parameters of SCD patients at baseline (week 8) and after both MAD-1 and MAD-2 dosing (i.e. after 8 weeks and at week 16) of Compound I according to the present disclosure. Elₘₐₓ is the maximal deformability or elongation index (informs red blood cell (“RBC”) cytoskeletal mechanics); Elₘᵢₙ corresponds to when RBCs can least elongate; and PoS refers to the point of sickling or point on the curve during deoxygenation when sickling begins.

FIG. 10 shows hemoximetry box plots of the partial pressure of O₂ (mm Hg) at which Hb is 20% saturated (p20) and 50% saturated (p50) with O₂ in blood samples of SCD patients after both MAD-1 and MAD-2 dosing of Compound I. Statistical significance was determined using Tukey's test. *P <0.05; **P<0.01; ****P<0.0001.

FIG. 11 shows the mean % Hb occupancy by Compound I in patients following MAD-1, MAD-2, and MAD-3 dosing. Hb occupancy data from Patient 0003 at 150 mg data point was excluded due to lack of adherence.

FIG. 12 shows hemoximetry box plots of the partial pressure of O₂ (mm Hg) at which Hb is 20% saturated (p20) and 50% saturated (p50) with O₂ in blood samples of SCD patients after MAD-3 dosing of Compound I. Statistical significance was determined using Tukey's test.

FIG. 13 shows Oxygenscan parameters of SCD patients at baseline and after 6 weeks of Compound I treatment according to the MAD-3 dosing regimen described in Example 3. Elₘₐₓ is the maximal deformability or elongation index (informs red blood cell (“RBC”) cytoskeletal mechanics); Elₘ in corresponds to when RBCs can least elongate. Point of sickling or point on the curve during deoxygenation refers to when sickling begins. AA blood is from HbAA genotype control.

FIG. 14 shows percentage of sickled red blood cells in SCD patients at baseline and after 6 weeks of MAD-3 dosing of Compound I. Baseline was at day 1 of MAD-3; patients were not on treatment. Patient 0001 (Patient #1) had a lower percentage of sickled cells at baseline.

FIG. 15 shows the observed percentage of hemoglobin occupancy at trough (mean and SD) compared with model predictions (shaded area) following a series of two first (loading) dose regimens administered over 2 days, followed by a daily second (maintenance) dose of Compound I (daily dose regimens of 50 mg QD and 100 mg QD, each administered over several weeks).

FIG. 16 shows the Study Schema for Part A of the study described in Example 5.

FIG. 17 shows the Study Schema for Part B of the study described in Example 5.

FIG. 18 shows the Study Schema for Part C of the study described in Example 5.

### DETAILED DESCRIPTION

### 1. Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as if it were individually recited herein.

Forms of Compound I or salts or solvates thereof are provided herein. In some embodiments, reference to a form of Compound I or a salt or a solvate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a salt or a solvate thereof present in a composition is in the designated form. For instance, in some embodiments, reference to Compound I Form I means that at least 50%, at least 55%, at least 60%, at least 65%, at least 70%, at least 75%, at least 80%, at least 85%, at least 90%, at least 95%, or at least 99% of Compound I present in a composition is in Form I.

The term “solid form” refers to a type of solid-state material that includes amorphous as well as crystalline forms. The term “crystalline form” refers to polymorphs as well as solvates, etc. The term “polymorph” refers to a particular crystal structure having particular physical properties such as X-ray diffraction, melting point, and the like.

The term “solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “desolvated” refers to a Compound I form that is a solvate as described herein, and from which solvent molecules have been partially or completely removed. Desolvation techniques to produce desolvated forms include, without limitation, exposure of a Compound I form (solvate) to a vacuum, subjecting the solvate to elevated temperature, exposing the solvate to a stream of gas, such as air or nitrogen, or any combination thereof. Thus, a desolvated Compound I form can be anhydrous, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

Any formula or structure given herein, including Compound I, is also intended to represent unlabeled forms as well as isotopically labeled forms of the compounds. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C, ¹³C, ¹⁴C; oxygen includes for example ¹⁶O, ¹⁷O, ¹⁸O; nitrogen includes for example ¹³N, ¹⁴N, ¹⁵N; sulfur includes for example 32S, 33S, 34S, ³⁵S, 365, 375, 385; fluoro includes for example ¹⁷F, ¹⁸F, ¹⁹F; chloro includes for example ³⁵Cl, ³⁶Cl, ³⁷Cl, ³⁸Cl, ³⁹Cl; and the like.

“Pharmaceutically acceptable” or “physiologically acceptable” refer to forms as described herein, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the forms described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like. In some embodiments, a pharmaceutically acceptable salt does not include a salt of a primary amine.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. A compound described herein, solid form described herein, or a salt or a solvate thereof, may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In some embodiments, the subject is a human.

The term “therapeutically effective amount” or “effective amount” of a compound or solid form described herein, or a salt or a solvate thereof, means an amount sufficient to effect treatment when administered to a subject, to provide a therapeutic benefit such as amelioration of symptoms or slowing of disease progression. For example, a therapeutically effective amount may be an amount sufficient to decrease a symptom of a sickle cell disease. The therapeutically effective amount may vary depending on the subject, and disease or condition being treated, the weight and age of the subject, the severity of the disease or condition, and the manner of administering, which can readily be determined by one of ordinary skill in the art.

In some embodiments, a therapeutically effective amount can also be ascertained by evaluating biomarkers of the disease or disorder to be treated. In the case of SCD, biomarkers may include measuring blood Hb levels, hemolysis (e.g., via blood bilirubin levels), percent Hb occupancy by compound, partial pressure of O₂ at which Hb is 20% saturated (p20) or 50% saturated (p50) with O₂, and RBC deformability as a function of oxygen tension (p02) including determination of maximum RBC deformability under normoxic conditions (EIₘₐₓ), minimum RBC deformability under hypoxic conditions (Elₘᵢₙ), and the specific PO₂ level at which RBC sickling occurs, i.e., the point of sickling (PoS). Therapeutic efficacy in SCD may also include measuring oxygen deliver to tissues including, e.g., microvasculature oxygen saturation, cerebral blood flow, oxygen extraction fraction, and cerebral metabolic rate for oxygen, which may be measured by, e.g., diffuse correlation spectroscopy (DSC) and/or frequency-domain near-infrared spectroscopy (FDNIRS).

In some embodiments, the therapeutically effective amount is determined by measuring the percent of hemoglobin occupied by the compound, i.e., the percent Hb occupancy. The percent Hb occupancy is a measure of target engagement defined as the percentage of hemoglobin bound by a compound (e.g., Compound I) calculated as the molar concentration of the compound (e.g., Compound I) in RBCs divided by the molar concentration of hemoglobin in RBCs (the mean corpuscular hemoglobin concentration calculated from the hematology panel). The values for concentration of a compound (e.g., Compound I) in RBCs is calculated from the concentrations in whole blood and plasma, and the hematocrit.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the forms described herein and compositions described herein may be used for a variety of purposes, including therapeutic and experimental purposes. For example, the forms described herein and compositions described herein may be used ex vivo to determine the optimal schedule and/or dosing of administration of a form of the present disclosure for a given indication, cell type, individual, and other parameters. Information gleaned from such use may be used for experimental purposes or in the clinic to set protocols for in vivo treatment. Other ex vivo uses for which the forms described herein and compositions described herein may be suited are described below or will become apparent to those skilled in the art. The selected forms described herein may be further characterized to examine the safety or tolerance dosage in human or non-human subjects. Such properties may be examined using commonly known methods to those skilled in the art.

The term “hemoglobin” as used herein refers to any hemoglobin protein, including normal hemoglobin (HbA) and abnormal hemoglobin, such as sickle hemoglobin (HbS).

The term “sickle cell disease” refers to diseases mediated by sickle hemoglobin (HbS) that results from a single point mutation in the hemoglobin (Hb). Sickle cell diseases include sickle cell anemia (HbSS), hemoglobin SC disease (HbSC), hemoglobin S beta-plus-thalassemia (HbS/β+) and hemoglobin S beta-zero-thalassemia (HbS/β0).

“First dose” refers to an initial dose of a compound as described herein, or series of such doses, given to achieve a target therapeutic level in the subject in a desired amount of time. In some embodiments, a “first dose” refers to a “loading dose”. In some embodiments, a “loading dose” refers to a “first dose”.

“Second dose” refers to a dose of a compound as described herein, or series of such doses, administered to maintain a desired therapeutic level of the compound in the subject. In some embodiments, a “second dose” refers to a “maintenance dose”. In some embodiments, a “maintenance dose” refers to a “second dose”.

A target therapeutic level can be assessed based on the percent hemoglobin occupancy or on parameters related thereto such as concentration of Compound I in the subject's blood relative to the subject's hematocrit.

### 2. Compound I and Methods of Use Thereof

Provided herein are methods for treating sickle cell disease (SCD). Sickle hemoglobin (HbS) contains a point mutation where glutamic acid is replaced with valine, making HbS susceptible to polymerization under hypoxic conditions to give the HbS containing red blood cells their characteristic sickle shape. The sickled cells are also more rigid than normal red blood cells, and their lack of flexibility can lead to blockage of blood vessels. It is contemplated that an approach to therapy would be to maintain the HbS in the oxygenated state, as polymerization occurs only in the deoxygenated state under hypoxic conditions.

Provided herein are methods for treating sickle cell disease (SCD) comprising administering (S)-2-hydroxy-6-((4-(2-(2-hydroxyethyl)nicotinoyl)morpholin-3-yl)methoxy)benzaldehyde (Compound I) or a pharmaceutically acceptable salt thereof. Compound I has the following formula:

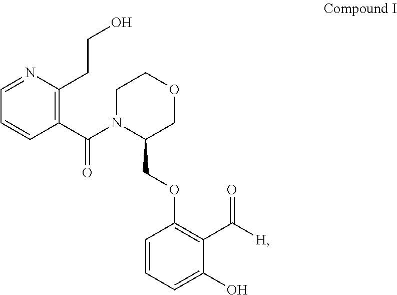

Compound I is a modulator of hemoglobin, and its synthesis and method of use thereof is described in U.S. Pat. No. 10,683,285.

In some embodiments, Compound I is a solid form.

In some embodiments, Compound I is a crystalline form. In some embodiments, Compound I is a crystalline form characterized by an X-ray powder diffractogram comprising the following peaks: 18.3, 23.4, and 26.1 °2θ±0.2 °2θ (Compound I Form I), as determined on a diffractometer using Cu-Kα radiation. In some embodiments, the diffractogram further comprises one or more peaks at: 10.8 or 17.3 °2θ±0.2 °2θ. In some embodiments, the crystalline form is characterized by a differential scanning calorimetry (DSC) curve that comprises an endotherm at about 111° C. (onset temperature).

Amorphous Compound I may be made according to methods known in the art. A solution of amorphous Compound I in acetonitrile (>540 mg/mL) was refrigerated for 4 days and then placed in a freezer for 1 day. The solids were filtered and dried under nitrogen to provide Compound I Form I.

Compound I Form I was also prepared as follows: Amorphous Compound I was slurried in ether with seeding with Compound I Form I from another experiment (prepared as described herein) at ambient temperature for 1 day, providing Compound I Form I.

X-ray Powder Diffraction (XRPD): XRPD figures were generated using SSCI Pattern Match 3.0.4, unvalidated software. XRPD patterns were collected with a PANalytical X′Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e or NIST SRM 640f) was analyzed to verify the observed position of the Si 111 peak is consistent with the NIST-certified position. A specimen of the sample was sandwiched between 3-gm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X′Celerator) located 240 mm from the specimen and Data Collector software v. 2.2b or v. 5.5.

Differential Scanning Calorimetry (DSC): DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to sample analysis. Samples were analyzed from −30° C. to 250° C. @10°/min.

Some embodiments provided herein comprise methods of administering Compound I or a pharmaceutically acceptable salt thereof. Some embodiments provided herein comprise methods of administering Compound I. Some embodiments provided herein comprise methods of administering a pharmaceutically acceptable salt of Compound I. In some embodiments, the dose of a pharmaceutically acceptable salt of Compound I is adjusted to be equivalent to the dose of Compound I.

Some embodiments provide for methods for treating sickle cell disease by administration of Compound I, or a pharmaceutically acceptable salt thereof, wherein the administration provides about 25% to about 80% hemoglobin occupancy. Some embodiments provide for methods for treating sickle cell disease by administration of Compound I, or a pharmaceutically acceptable salt thereof, wherein the administration provides about 20% to about 60% hemoglobin occupancy.

Some embodiments provide for methods for treating sickle cell disease by administration of Compound I, or a pharmaceutically acceptable salt thereof, wherein the administration provides about 30% to about 50% hemoglobin occupancy.

It is contemplated that the methods described herein can achieve targeted hemoglobin occupancy and attain desired hematological effects, therefore improving clinical outcomes (such as numerically fewer vaso-occlusive crises) for individuals living with SCD.

Some embodiments provide for methods for treating sickle cell disease in a subject in need thereof, comprising administering to the subject a first dose of Compound I of formula:

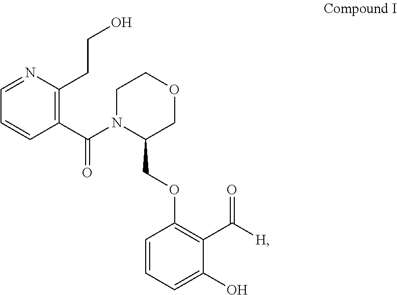

or a pharmaceutically acceptable salt thereof, for one, two, three, four, five, six, or seven days, followed by a second dose of Compound I, or a pharmaceutically acceptable salt thereof, thereafter, wherein the administrations of the first dose and the second dose provide about 25% to about 80% hemoglobin occupancy by Compound I.

Some embodiments provide for methods for treating sickle cell disease in a subject in need thereof, comprising administering to the subject a first dose of Compound I of formula:

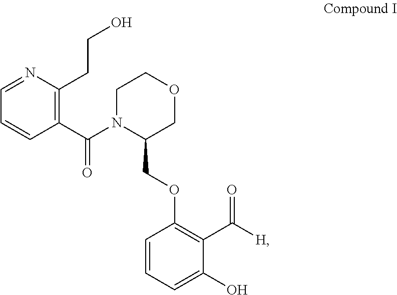

or a pharmaceutically acceptable salt thereof, for one, two, three, four, five, six, or seven days, followed by a second dose of Compound I, or a pharmaceutically acceptable salt thereof, thereafter, wherein the administrations of the first dose and the second dose provide about 25% to about 60% hemoglobin occupancy by Compound I.

Some embodiments provide for methods for treating sickle cell disease in a subject in need thereof, comprising administering to the subject a first dose of Compound I of formula:

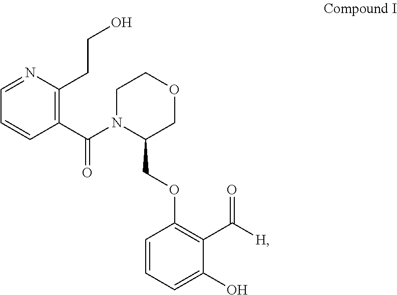

or a pharmaceutically acceptable salt thereof, for one, two, three, four, five, six, or seven days, followed by a second dose of Compound I, or a pharmaceutically acceptable salt thereof, wherein the administration of the first dose and the second dose provide about 30% to about 50% hemoglobin occupancy by Compound I.

In some embodiments, the percent hemoglobin occupancy, as provided by the administration of Compound I, is about 25% to about 80%. In some embodiments, the percent hemoglobin occupancy, as provided by the administration of Compound I, is about 25% to about 75%. In some embodiments, the percent hemoglobin occupancy, as provided by the administration of Compound I, is about 25% to about 70%. In some embodiments, the percent hemoglobin occupancy, as provided by the administration of Compound I, is about 25% to about 65%. In some embodiments, the percent hemoglobin occupancy, as provided by the administration of Compound I, is about 25% to about 60%. In some embodiments, the percent hemoglobin occupancy is about 25% to about 55%.

In some embodiments, the percent hemoglobin occupancy (as provided by the administration of Compound I) is about 25% to about 50%. In some embodiments, the percent hemoglobin occupancy is about 25% to about 45%. In some embodiments, the percent hemoglobin occupancy is about 30% to about 60%. In some embodiments, the percent hemoglobin occupancy is about 30% to about 55%. In some embodiments, the percent hemoglobin occupancy is about 30% to about 50%. In some embodiments, the percent hemoglobin occupancy is about 30% to about 45%. In some embodiments, the percent hemoglobin occupancy is about 30% to about 40%. In some embodiments, the percent hemoglobin occupancy is about 30% to about 35%. In some embodiments, the percent hemoglobin occupancy is about 35% to about 60%. In some embodiments, the percent hemoglobin occupancy is about 35% to about 55%. In some embodiments, the percent hemoglobin occupancy is about 35% to about 50%. In some embodiments, the percent hemoglobin occupancy is about 35% to about 40%. In some embodiments, the percent hemoglobin occupancy is about 40% to about 60%. In some embodiments, the percent hemoglobin occupancy is about 40% to about 55%. In some embodiments, the percent hemoglobin occupancy is about 40% to about 50%. In some embodiments, the percent hemoglobin occupancy is about 40% to about 45%.

In some embodiments, the percent hemoglobin occupancy may be calculated as described herein. In some embodiments, percent hemoglobin occupancy is the molar ratio of Compound I concentration to the Hb concentration in red blood cells.

Some embodiments provide for methods for treating sickle cell disease in a subject in need thereof, comprising administering to the subject a first dose of Compound I, or a pharmaceutically acceptable salt thereof, for one, two, three, four, five, six, or seven days to achieve a target therapeutic level in the subject, followed by a second dose of Compound I, or a pharmaceutically acceptable salt thereof, thereafter to maintain the target therapeutic level in the subject, wherein on a daily dose basis the first dose is greater than the second dose.

Some embodiments provide for methods for treating sickle cell disease in a subject in need thereof, comprising administering to the subject a first dose of Compound I, or a pharmaceutically acceptable salt thereof, for one, two, three, four, five, six, or seven days to achieve a target therapeutic level in the subject, followed thereafter by a second dose of Compound I, or a pharmaceutically acceptable salt thereof, to maintain the target therapeutic level in the subject, wherein on a daily dose basis the first dose is greater than the second dose (i.e. the first dose per day is greater than the second dose per day).

In some embodiments, the ratio of first dose to second dose is about 88:1 to about 2:1. In some embodiments, the ratio of first dose to second dose is about 60:1 to about 2:1. In some embodiments, the ratio of first dose to second dose is about 40:1 to about 2:1. In some embodiments, the ratio of first dose to second dose is about 20:1 to about 2:1. In some embodiments, the ratio of first dose to second dose is about to second dose 10:1 to about 2:1.

In some embodiments, the ratio of first dose per day to second dose per day is about 88:1 to about 2:1. In some embodiments, the ratio of first dose per day to second dose per day is about 60:1 to about 2:1. In some embodiments, the ratio of first dose per day to second dose per day is about 40:1 to about 2:1. In some embodiments, the ratio of first dose per day to second dose per day is about 20:1 to about 2:1. In some embodiments, the ratio of first dose per day to second dose per day is about 10:1 to about 2:1. In some embodiments, the ratio of first dose per day to second dose per day is about 4:1 to about 2:1. In some embodiments, the ratio of first dose per day to second dose per day is about 9:1 to about 4:1.

Some embodiments provide for methods for treating sickle cell disease in a subject in need thereof, comprising administering to the subject a first dose of about 100 mg per day to about 2200 mg per day of Compound I, or a pharmaceutically acceptable salt thereof, for one, two, three, four, five, six, or seven days, followed thereafter by a second dose of about 25 mg per day to about 500 mg per day of Compound I, or a pharmaceutically acceptable salt thereof.

Some embodiments provide for methods for treating sickle cell disease in a subject in need thereof, comprising administering to the subject a first dose of about 200 mg per day to about 1600 mg per day of Compound I, or a pharmaceutically acceptable salt thereof, for one, two, three, four, five, six, or seven days, followed thereafter by a second dose of about 25 mg per day to about 500 mg per day of Compound I, or a pharmaceutically acceptable salt thereof.

Some embodiments provide for methods for treating sickle cell disease in a subject in need thereof, comprising administering to the subject a first dose of about 300 mg per day to about 1500 mg per day of Compound I, or a pharmaceutically acceptable salt thereof, for one, two, three, four, five, six, or seven days, followed thereafter by a second dose of about 25 mg per day to about 500 mg per day of Compound I, or a pharmaceutically acceptable salt thereof.

Some embodiments provide for methods for treating sickle cell disease in a subject in need thereof, comprising administering to the subject a first dose of about 300 mg per day to about 1500 mg per day of Compound I, or a pharmaceutically acceptable salt thereof, for one, two, three, four, five, six, or seven days, followed thereafter by a second dose of about 25 mg per day to about 250 mg per day of Compound I, or a pharmaceutically acceptable salt thereof.

Some embodiments provide for methods for treating sickle cell disease in a subject in need thereof, comprising administering to the subject a first dose of about 300 mg per day to about 900 mg per day of Compound I, or a pharmaceutically acceptable salt thereof, for one, two, three, four, five, six, or seven days, followed thereafter by a second dose of about 25 mg per day to about 250 mg per day of Compound I, or a pharmaceutically acceptable salt thereof.

Some embodiments provide for methods for treating sickle cell disease in a subject in need thereof, comprising administering to the subject a first dose of about 200 mg per day to about 900 mg per day of Compound I, or a pharmaceutically acceptable salt thereof, for one, two, three, four, five, six, or seven days, followed by a second dose of about 25 mg per day to about 250 mg per day of Compound I, or a pharmaceutically acceptable salt thereof, thereafter.

Some embodiments provide for methods for treating sickle cell disease in a subject in need thereof, comprising administering to the subject a first dose of about 200 mg per day to about 900 mg per day of Compound I, or a pharmaceutically acceptable salt thereof, for one, two, three, four, five, six, or seven days, followed thereafter by a second dose of about 25 mg per day to about 200 mg per day of Compound I, or a pharmaceutically acceptable salt thereof.

Some embodiments provide for methods for treating sickle cell disease in a subject in need thereof, comprising administering to the subject a first dose of about 200 mg per day to about 900 mg per day of Compound I, or a pharmaceutically acceptable salt thereof, for one, two, three, four, five, six, or seven days, followed thereafter by a second dose of about 25 mg per day to about 150 mg per day of Compound I, or a pharmaceutically acceptable salt thereof.

Some embodiments provide for methods for treating sickle cell disease in a subject in need thereof, comprising administering to the subject a first dose of about 200 mg per day to about 1600 mg per day of Compound I, or a pharmaceutically acceptable salt thereof, for one to five days, followed by a second dose of about 50 mg per day to about 500 mg per day of Compound I, or a pharmaceutically acceptable salt thereof.

Some embodiments provide for methods for treating sickle cell disease in a subject in need thereof, comprising administering to the subject a first dose of about 300 mg per day to about 1500 mg per day of Compound I, or a pharmaceutically acceptable salt thereof, for one to five days, followed by a second dose of about 50 mg per day to about 500 mg per day of Compound I, or a pharmaceutically acceptable salt thereof.

Some embodiments provide for methods for treating sickle cell disease in a subject in need thereof, comprising administering to the subject a first dose of about 200 mg per day to about 900 mg per day of Compound I, or a pharmaceutically acceptable salt thereof, for one to five days, followed by a second dose of about 50 mg per day to about 250 mg per day of Compound I, or a pharmaceutically acceptable salt thereof.

In some embodiments, the first dose is administered for one to seven days. In some embodiments, the first dose is administered for one to six days. In some embodiments, the first dose is administered for one to five days. In some embodiments, the first dose is administered for one to four days. In some embodiments, the first dose is administered for one to three days. In some embodiments, the first dose is administered for one to two days.

Some embodiments provide for methods for treating sickle cell disease in a subject in need thereof, comprising administering to the subject a first dose of about 300 mg per day to about 900 mg per day of Compound I, or a pharmaceutically acceptable salt thereof, for one to three days, followed by a second dose of about 50 mg per day to about 250 mg per day of Compound I, or a pharmaceutically acceptable salt thereof.

In some embodiments, the first dose comprises about 100 mg per day to about 2200 mg per day of Compound I, or a pharmaceutically acceptable salt thereof. In some embodiments, the first dose comprises about 200 mg per day to about 2000 mg per day of Compound I, or a pharmaceutically acceptable salt thereof. In some embodiments, the first dose comprises about 200 mg per day to about 1800 mg per day of Compound I, or a pharmaceutically acceptable salt thereof.

In some embodiments, the first dose comprises about 200 mg per day to about 1600 mg per day of Compound I, or a pharmaceutically acceptable salt thereof. In some embodiments, the first dose comprises about 300 mg per day to about 1500 mg per day of Compound I, or a pharmaceutically acceptable salt thereof. In some embodiments, the first dose comprises about 200 mg per day to about 1500 mg per day of Compound I, or a pharmaceutically acceptable salt thereof. In some embodiments, the first dose comprises about 200 mg per day to about 1000 mg per day of Compound I, or a pharmaceutically acceptable salt thereof. In some embodiments, the first dose comprises about 200 mg per day to about 900 mg per day of Compound I, or a pharmaceutically acceptable salt thereof.

In some embodiments, the first dose comprises about 1500 mg per day of Compound I, or a pharmaceutically acceptable salt thereof. In some embodiments, the first dose comprises about 1400 mg per day of Compound I, or a pharmaceutically acceptable salt thereof. In some embodiments, the first dose comprises about 1300 mg per day of Compound I, or a pharmaceutically acceptable salt thereof. In some embodiments, the first dose comprises about 1200 mg per day of Compound I, or a pharmaceutically acceptable salt thereof. In some embodiments, the first dose comprises about 1100 mg per day of Compound I, or a pharmaceutically acceptable salt thereof. In some embodiments, the first dose comprises about 1000 mg per day of Compound I, or a pharmaceutically acceptable salt thereof. In some embodiments, the first dose comprises about 900 mg per day of Compound I, or a pharmaceutically acceptable salt thereof. In some embodiments, the first dose comprises about 800 mg per day of Compound I, or a pharmaceutically acceptable salt thereof. In some embodiments, the first dose comprises about 700 mg per day of Compound I, or a pharmaceutically acceptable salt thereof. In some embodiments, the first dose comprises about 600 mg per day of Compound I, or a pharmaceutically acceptable salt thereof. In some embodiments, the first dose comprises about 500 mg per day of Compound I, or a pharmaceutically acceptable salt thereof. In some embodiments, the first dose comprises about 400 mg per day of Compound I, or a pharmaceutically acceptable salt thereof. In some embodiments, the first dose comprises about 300 mg per day of Compound I, or a pharmaceutically acceptable salt thereof. In some embodiments, the first dose comprises about 200 mg per day of Compound I, or a pharmaceutically acceptable salt thereof. In some embodiments, the first dose comprises about 100 mg per day of Compound I, or a pharmaceutically acceptable salt thereof.

In some embodiments, the first dose is administered for one to seven days. In some embodiments, the first dose is administered for one to six days. In some embodiments, the first dose is administered for one to five days. In some embodiments, the first dose is administered for one to four days. In some embodiments, the first dose is administered for one to three days. In some embodiments, the first dose is administered for one to two days.

In some embodiments, the first dose is administered for seven days. In some embodiments, the first dose is administered for six days. In some embodiments, the first dose is administered for five days. In some embodiments, the first dose is administered for four days. In some embodiments, the first dose is administered for three days. In some embodiments, the first dose is administered for two days. In some embodiments, the first dose is administered for one day.

In some embodiments, the first dose is administered for one, two, three, or four days. In some embodiments, the first dose is administered for one, two, or three days.

In some embodiments, the first dose comprises:
about 600 mg per day of Compound I, or a pharmaceutically acceptable salt thereof, administered for one day;
followed thereafter by about 500 mg per day of Compound I, or a pharmaceutically acceptable salt thereof, administered for one day or two days.

In some embodiments, the first dose is:
about 700 mg per day of Compound I, or a pharmaceutically acceptable salt thereof, administered for one day;
followed thereafter by about 700 mg per day of Compound I, or a pharmaceutically acceptable salt thereof, administered for one day or two days.

In some embodiments, the first dose is:
about 800 mg per day of Compound I, or a pharmaceutically acceptable salt thereof, administered for one day;
followed thereafter by about 700 mg per day of Compound I, or a pharmaceutically acceptable salt thereof, administered for one day or two days.

In some embodiments, the first dose is: about 900 mg per day of Compound I, or a pharmaceutically acceptable salt thereof, administered for one day;
followed thereafter by about 800 mg per day of Compound I, or a pharmaceutically acceptable salt thereof, administered for one day or two days.

In some embodiments, the first dose is:
about 900 mg per day of Compound I, or a pharmaceutically acceptable salt thereof, administered for at least one day;
followed thereafter by about 800 mg per day of Compound I, or a pharmaceutically acceptable salt thereof, administered for one, two, three, or four days.

In some embodiments, the first dose is:
about 900 mg per day of Compound I, or a pharmaceutically acceptable salt thereof, administered for one day;
followed thereafter by about 800 mg per day of Compound I, or a pharmaceutically acceptable salt thereof, administered for three days.

In some embodiments, the first dose is:
about 500 mg per day of Compound I, or a pharmaceutically acceptable salt thereof, administered for one day;
followed thereafter by about 400 mg per day of Compound I, or a pharmaceutically acceptable salt thereof, administered for one day or two days.

In some embodiments, the first dose is:
about 1000 mg per day of Compound I, or a pharmaceutically acceptable salt thereof, administered for one day;
followed thereafter by about 900 mg per day of Compound I, or a pharmaceutically acceptable salt thereof, administered for one day or two days.

In some embodiments, the first dose is:
about 300 mg per day of Compound I, or a pharmaceutically acceptable salt thereof, administered for one day;
followed thereafter by about 200 mg per day of Compound I, or a pharmaceutically acceptable salt thereof, administered for one day or two days.

In some embodiments, the first dose is:
about 600 mg per day of Compound I, or a pharmaceutically acceptable salt thereof, administered for one day;
followed thereafter by about 500 mg per day of Compound I, or a pharmaceutically acceptable salt thereof, administered for one day.

In some embodiments, the first dose is:
about 700 mg per day of Compound I, or a pharmaceutically acceptable salt thereof, administered for one day;
followed thereafter by about 700 mg per day of Compound I, or a pharmaceutically acceptable salt thereof, administered for one day.

In some embodiments, the first dose is:
about 800 mg per day of Compound I, or a pharmaceutically acceptable salt thereof, administered for one day;
followed thereafter by about 700 mg per day of Compound I, or a pharmaceutically acceptable salt thereof, administered for one day.

In some embodiments, the first dose is:
about 900 mg per day of Compound I, or a pharmaceutically acceptable salt thereof, administered for one day;
followed thereafter by about 800 mg per day of Compound I, or a pharmaceutically acceptable salt thereof, administered for one day.

In some embodiments, the first dose is:
about 900 mg per day of Compound I, or a pharmaceutically acceptable salt thereof, administered for one day;
followed by about 800 mg per day of Compound I, or a pharmaceutically acceptable salt thereof, administered for one, two, or three days.

In some embodiments, the first dose is:
about 500 mg per day of Compound I, or a pharmaceutically acceptable salt thereof, administered for one day;
followed thereafter by about 400 mg per day of Compound I, or a pharmaceutically acceptable salt thereof, administered for one day.

In some embodiments, the first dose is:
about 1000 mg per day of Compound I, or a pharmaceutically acceptable salt thereof, administered for one day;
followed thereafter by about 900 mg per day of Compound I, or a pharmaceutically acceptable salt thereof, administered for one day.

In some embodiments, the first dose is:
about 300 mg per day of Compound I, or a pharmaceutically acceptable salt thereof, administered for one day;
followed thereafter by about 200 mg per day of Compound I, or a pharmaceutically acceptable salt thereof, administered for one day.

In some embodiments, the first dose is:
about 600 mg per day of Compound I, or a pharmaceutically acceptable salt thereof, for one day;
followed by about 500 mg per day of Compound I, or a pharmaceutically acceptable salt thereof, for one day.

In some embodiments, the first dose is:
about 700 mg per day of Compound I, or a pharmaceutically acceptable salt thereof, for one day;
followed by about 700 mg per day of Compound I, or a pharmaceutically acceptable salt thereof, for one day.

In some embodiments, the first dose is:
about 800 mg per day of Compound I, or a pharmaceutically acceptable salt thereof, for one day;
followed by about 700 mg per day of Compound I, or a pharmaceutically acceptable salt thereof, for one day.

In some embodiments, the first dose is:
about 900 mg per day of Compound I, or a pharmaceutically acceptable salt thereof, for one day;
followed by about 800 mg per day of Compound I, or a pharmaceutically acceptable salt thereof, for one day.

In some embodiments, the first dose is:
about 500 mg per day of Compound I, or a pharmaceutically acceptable salt thereof, for one day;
followed by about 400 mg per day of Compound I, or a pharmaceutically acceptable salt thereof, for one day.

In some embodiments, the first dose is:
about 1000 mg per day of Compound I, or a pharmaceutically acceptable salt thereof, for one day;
followed by about 900 mg per day of Compound I, or a pharmaceutically acceptable salt thereof, for one day.

In some embodiments, the first dose is:
about 300 mg per day of Compound I, or a pharmaceutically acceptable salt thereof, for one day;
followed by about 200 mg per day of Compound I, or a pharmaceutically acceptable salt thereof, for one day.

In some embodiments of the methods described herein, the method comprises administering a first dose of about 50 mg twice daily (BID) of Compound I, or a pharmaceutically acceptable salt thereof, for one, two, three, four, five, six, or seven days. In some embodiments, the method comprises administering a first dose of about 100 mg per day of Compound I, or a pharmaceutically acceptable salt thereof, for one, two, three, four, five, six, or seven days.

In some embodiments, the method comprises administering a first dose of about 75 mg twice daily (BID) of Compound I, or a pharmaceutically acceptable salt thereof, for one, two, three, four, five, six, or seven days. In some embodiments, the method comprises administering a first dose of about 150 mg per day of Compound I, or a pharmaceutically acceptable salt thereof, for one, two, three, four, five, six, or seven days.

In some embodiments, the method comprises administering a first dose of about 100 mg twice daily (BID) of Compound I, or a pharmaceutically acceptable salt thereof, for one, two, three, four, five, six, or seven days. In some embodiments, the method comprises administering a first dose of about 200 mg per day of Compound I, or a pharmaceutically acceptable salt thereof, for one, two, three, four, five, six, or seven days.

In some embodiments, the method comprises administering a first dose of about 150 mg twice daily (BID) of Compound I, or a pharmaceutically acceptable salt thereof, for one, two, three, four, five, six, or seven days. In some embodiments, the method comprises administering a first dose of about 300 mg per day of Compound I, or a pharmaceutically acceptable salt thereof, for one, two, three, four, five, six, or seven days.

In some embodiments, the method comprises administering a first dose of about 200 mg twice daily (BID) of Compound I, or a pharmaceutically acceptable salt thereof, for one, two, three, four, five, six, or seven days. In some embodiments, the method comprises administering a first dose of about 400 mg per day of Compound I, or a pharmaceutically acceptable salt thereof, for one, two, three, four, five, six, or seven days.

In some embodiments, the method comprises administering a first dose of about 250 mg twice daily (BID) of Compound I, or a pharmaceutically acceptable salt thereof, for one, two, three, four, five, six, or seven days. In some embodiments, the method comprises administering a first dose of about 500 mg per day of Compound I, or a pharmaceutically acceptable salt thereof, for one, two, three, four, five, six, or seven days.

In some embodiments, the method comprises administering a first dose of about 150 mg four times daily (QID) of Compound I, or a pharmaceutically acceptable salt thereof, for one, two, three, four, five, six, or seven days. In some embodiments, the method comprises administering a first dose of about 150 mg every six hours (Q6H) of Compound I, or a pharmaceutically acceptable salt thereof, for one, two, three, four, five, six, or seven days. In some embodiments, the method comprises administering a first dose of about 200 mg three times a day (TID) of Compound I, or a pharmaceutically acceptable salt thereof, for one, two, three, four, five, six, or seven days. In some embodiments, the method comprises administering a first dose of about 300 mg twice a day (BID) of Compound I, or a pharmaceutically acceptable salt thereof, for one, two, three, four, five, six, or seven days. In some embodiments, the method comprises administering a first dose of about 600 mg per day of Compound I, or a pharmaceutically acceptable salt thereof, for one, two, three, four, five, six, or seven days.

In some embodiments, the method comprises administering a first dose of about 150 mg four times daily (QID) of Compound I, or a pharmaceutically acceptable salt thereof, for four days. In some embodiments, the method comprises administering a first dose of about 150 mg every six hours (Q6H) of Compound I, or a pharmaceutically acceptable salt thereof, for four days. In some embodiments, the method comprises administering a first dose of about 200 mg three times a day (TID) of Compound I, or a pharmaceutically acceptable salt thereof, for four days. In some embodiments, the method comprises administering a first dose of about 300 mg twice a day (BID) of Compound I, or a pharmaceutically acceptable salt thereof, for four days. In some embodiments, the method comprises administering a first dose of about 600 mg per day of Compound I, or a pharmaceutically acceptable salt thereof, for four days.

In some embodiments, the method comprises administering a first dose of about 350 mg twice daily (BID) of Compound I, or a pharmaceutically acceptable salt thereof, for one, two, three, four, five, six, or seven days. In some embodiments, the method comprises administering a first dose of about 700 mg per day of Compound I, or a pharmaceutically acceptable salt thereof, for one, two, three, four, five, six, or seven days.

In some embodiments, the method comprises administering a first dose of about 400 mg twice daily (BID) of Compound I, or a pharmaceutically acceptable salt thereof, for one, two, three, four, five, six, or seven days. In some embodiments, the method comprises administering a first dose of about 800 mg per day of Compound I, or a pharmaceutically acceptable salt thereof, for one, two, three, four, five, six, or seven days.

In some embodiments, the method comprises administering a first dose of about 450 mg twice daily (BID) of Compound I, or a pharmaceutically acceptable salt thereof, for one, two, three, four, five, six, or seven days. In some embodiments, the method comprises administering a first dose of about 900 mg per day of Compound I, or a pharmaceutically acceptable salt thereof, for one, two, three, four, five, six, or seven days.

In some embodiments, the method comprises administering a first dose of about 500 mg twice daily (BID) of Compound I, or a pharmaceutically acceptable salt thereof, for one, two, three, four, five, six, or seven days. In some embodiments, the method comprises administering a first dose of about 1000 mg per day of Compound I, or a pharmaceutically acceptable salt thereof, for one, two, three, four, five, six, or seven days.

Following a first dose administered over one, two, three, four, five, six, or seven days to achieve a target therapeutic level of Compound I, or a pharmaceutically acceptable salt thereof, in the subject, a second dose of Compound I, or a pharmaceutically acceptable salt thereof, is administered thereafter to maintain the target therapeutic level in the subject. In some embodiments, the therapeutic level of Compound I is the determined by measuring the percent hemoglobin occupied, or % Hb occupancy, by Compound I.

In some embodiments, the second dose is about 25 mg per day to about 300 mg per day of Compound I, or a pharmaceutically acceptable salt thereof.

In some embodiments, the second dose is about 25 mg per day to about 275 mg per day of Compound I, or a pharmaceutically acceptable salt thereof.

In some embodiments, the second dose is about 25 mg per day to about 250 mg per day of Compound I, or a pharmaceutically acceptable salt thereof.

In some embodiments, the second dose is about 25 mg per day to about 225 mg per day of Compound I, or a pharmaceutically acceptable salt thereof.

In some embodiments, the second dose is about 25 mg per day to about 200 mg per day of Compound I, or a pharmaceutically acceptable salt thereof.

In some embodiments, the second dose is about 25 mg per day to about 175 mg per day of Compound I, or a pharmaceutically acceptable salt thereof.

In some embodiments, the second dose is about 25 mg per day to about 150 mg per day of Compound I, or a pharmaceutically acceptable salt thereof.

In some embodiments, the second dose is about 25 mg per day to about 100 mg per day of Compound I, or a pharmaceutically acceptable salt thereof.

In some embodiments, the second dose is about 25 mg per day to about 75 mg per day of Compound I, or a pharmaceutically acceptable salt thereof.

In some embodiments, the second dose is about 25 mg per day to about 50 mg per day of Compound I, or a pharmaceutically acceptable salt thereof.

In some embodiments, the second dose is about 50 mg per day to about 150 mg per day of Compound I, or a pharmaceutically acceptable salt thereof.

In some embodiments, the second dose is about 50 mg per day to about 100 mg per day of Compound I, or a pharmaceutically acceptable salt thereof.

In some embodiments, the second dose is about 25 mg per day to about 600 mg per day of Compound I, or a pharmaceutically acceptable salt thereof. In some embodiments, the second dose is about 25 mg per day to about 550 mg per day of Compound I, or a pharmaceutically acceptable salt thereof. In some embodiments, the second dose is about 25 mg per day to about 500 mg per day of Compound I, or a pharmaceutically acceptable salt thereof. In some embodiments, the second dose is about 25 mg per day to about 450 mg per day of Compound I, or a pharmaceutically acceptable salt thereof. In some embodiments, the second dose is about 25 mg per day to about 400 mg per day of Compound I, or a pharmaceutically acceptable salt thereof. In some embodiments, the second dose is about 25 mg per day to about 350 mg per day of Compound I, or a pharmaceutically acceptable salt thereof. In some embodiments, the second dose is about 25 mg per day to about 300 mg per day of Compound I, or a pharmaceutically acceptable salt thereof.

In some embodiments, the second dose is about 25 mg per day of Compound I, or a pharmaceutically acceptable salt thereof. In some embodiments, the second dose is about 50 mg per day of Compound I, or a pharmaceutically acceptable salt thereof. In some embodiments, the second dose is about 75 mg per day of Compound I, or a pharmaceutically acceptable salt thereof. In some embodiments, the second dose is about 100 mg per day of Compound I, or a pharmaceutically acceptable salt thereof. In some embodiments, the second dose is about 125 mg per day of Compound I, or a pharmaceutically acceptable salt thereof. In some embodiments, the second dose is about 150 mg per day of Compound I, or a pharmaceutically acceptable salt thereof. In some embodiments, the second dose is about 175 mg per day of Compound I, or a pharmaceutically acceptable salt thereof. In some embodiments, the second dose is about 200 mg per day of Compound I, or a pharmaceutically acceptable salt thereof. In some embodiments, the second dose is about 225 mg per day of Compound I, or a pharmaceutically acceptable salt thereof. In some embodiments, the second dose is about 250 mg per day of Compound I, or a pharmaceutically acceptable salt thereof. In some embodiments, the second dose is about 275 mg per day of Compound I, or a pharmaceutically acceptable salt thereof. In some embodiments, the second dose is about 300 mg per day of Compound I, or a pharmaceutically acceptable salt thereof. In some embodiments, the second dose is about 350 mg per day of Compound I, or a pharmaceutically acceptable salt thereof. In some embodiments, the second dose is about 400 mg per day of Compound I, or a pharmaceutically acceptable salt thereof. In some embodiments, the second dose is about 450 mg per day of Compound I, or a pharmaceutically acceptable salt thereof. In some embodiments, the second dose is about 500 mg per day of Compound I, or a pharmaceutically acceptable salt thereof.

In some embodiments, the second dose is administered once daily (QD).

In some embodiments, the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 100 mg per day of Compound I, or a pharmaceutically acceptable salt thereof.

In some embodiments, the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 150 mg per day of Compound I, or a pharmaceutically acceptable salt thereof.

In some embodiments, the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 200 mg per day of Compound I, or a pharmaceutically acceptable salt thereof.

Some embodiments provide for methods for treating sickle cell disease in a subject in need thereof, comprising administering to the subject a first dose of Compound I, or a pharmaceutically acceptable salt thereof, wherein:
the first dose is:
   about 500 mg per day of Compound I, or a pharmaceutically acceptable salt thereof, administered for one day;
   followed by about 400 mg per day of Compound I, or a pharmaceutically acceptable salt thereof, administered for one day; and
   followed thereafter by a second dose of about 150 mg per day of Compound I, or a pharmaceutically acceptable salt thereof.

In some embodiments, the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 175 mg per day of Compound I, or a pharmaceutically acceptable salt thereof.

In some embodiments, the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 125 mg per day of Compound I, or a pharmaceutically acceptable salt thereof.

In some embodiments, the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 75 mg per day of Compound I, or a pharmaceutically acceptable salt thereof.

In some embodiments, the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 50 mg per day of Compound I, or a pharmaceutically acceptable salt thereof.

In some embodiments, the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 200 mg per day of Compound I, or a pharmaceutically acceptable salt thereof.

In some embodiments, the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 175 mg per day of Compound I, or a pharmaceutically acceptable salt thereof.

In some embodiments, the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 150 mg per day of Compound I, or a pharmaceutically acceptable salt thereof.

In some embodiments, the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 125 mg per day of Compound I, or a pharmaceutically acceptable salt thereof.

In some embodiments, the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 100 mg per day of Compound I, or a pharmaceutically acceptable salt thereof.

Some embodiments provide for methods for treating sickle cell disease in a subject in need thereof, comprising administering to the subject a first dose of Compound I, or a pharmaceutically acceptable salt thereof, wherein:
the first dose is:
   about 500 mg per day of Compound I, or a pharmaceutically acceptable salt thereof, administered for one day;
   followed by about 400 mg per day of Compound I, or a pharmaceutically acceptable salt thereof, administered for one day; and
   followed thereafter by a second dose of about 100 mg per day of Compound I, or a pharmaceutically acceptable salt thereof.

In some embodiments, the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 75 mg per day of Compound I, or a pharmaceutically acceptable salt thereof.

In some embodiments, the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 50 mg per day of Compound I, or a pharmaceutically acceptable salt thereof.

Some embodiments provide for methods for treating sickle cell disease in a subject in need thereof, comprising administering to the subject a first dose of Compound I, or a pharmaceutically acceptable salt thereof, wherein:
the first dose is:
   about 300 mg per day of Compound I, or a pharmaceutically acceptable salt thereof, administered for one day;
   followed by about 200 mg per day of Compound I, or a pharmaceutically acceptable salt thereof, administered for one day; and
   followed thereafter by a second dose of about 50 mg per day of Compound I, or a pharmaceutically acceptable salt thereof.

In some embodiments, the first dose is:
about 600 mg per day of Compound I, or a pharmaceutically acceptable salt thereof, administered for one day;
followed by about 500 mg per day of Compound I, or a pharmaceutically acceptable salt thereof, administered for one day; and
followed thereafter by a second dose of about 75 mg per day of Compound I, or a pharmaceutically acceptable salt thereof.

In some embodiments, the first dose is:
about 700 mg per day of Compound I, or a pharmaceutically acceptable salt thereof, administered for one day;
followed by about 700 mg per day of Compound I, or a pharmaceutically acceptable salt thereof, administered for one day; and
followed thereafter by a second dose of about 100 mg per day of Compound I, or a pharmaceutically acceptable salt thereof.

In some embodiments, the first dose is:
about 800 mg per day of Compound I, or a pharmaceutically acceptable salt thereof, administered for one day;
followed by about 700 mg per day of Compound I, or a pharmaceutically acceptable salt thereof, administered for one day; and
followed thereafter by a second dose of about 150 mg per day of Compound I, or a pharmaceutically acceptable salt thereof.

In some embodiments, the first dose is:
about 800 mg per day of Compound I, or a pharmaceutically acceptable salt thereof, administered for one day;
followed by about 700 mg per day of Compound I, or a pharmaceutically acceptable salt thereof, administered for one day; and
followed thereafter by a second dose of about 125 mg per day of Compound I, or a pharmaceutically acceptable salt thereof.

In some embodiments, the first dose is:
about 100 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 100 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 100 mg per day of Compound I, or a pharmaceutically acceptable salt thereof, administered for four, five, six, or seven days; and
followed thereafter by a second dose of about 25 mg per day of Compound I, or a pharmaceutically acceptable salt thereof.

In some embodiments, the first dose is:
about 150 mg per day of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 150 mg per day of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 150 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 150 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 150 mg per day of Compound I, or a pharmaceutically acceptable salt thereof, administered for four, five, six, or seven days; and
followed thereafter by a second dose of about 25 mg per day of Compound I, or a pharmaceutically acceptable salt thereof.

In some embodiments, the first dose is:
about 200 mg per day of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 200 mg per day of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 200 mg per day of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 200 mg per day of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 200 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 200 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 200 mg per day of Compound I, or a pharmaceutically acceptable salt thereof, administered for four, five, six, or seven days; and
followed thereafter by a second dose of about 25 mg per day of Compound I, or a pharmaceutically acceptable salt thereof.

In some embodiments, the first dose is:
about 400 mg per day of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 400 mg per day of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 400 mg per day of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 400 mg per day of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 400 mg per day of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 400 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 400 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 200 mg twice daily (BID) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 200 mg twice daily (BID) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 200 mg twice daily (BID) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 200 mg twice daily (BID) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 200 mg twice daily (BID) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 200 mg twice daily (BID) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 200 mg twice daily (BID) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 600 mg per day of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 600 mg per day of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 600 mg per day of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 600 mg per day of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 600 mg per day of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 600 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 600 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 300 mg twice daily (BID) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 300 mg twice daily (BID) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 300 mg twice daily (BID) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 300 mg twice daily (BID) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 300 mg twice daily (BID) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 300 mg twice daily (BID) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 300 mg twice daily (BID) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is
about 200 mg three times daily (TID) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 200 mg three times daily (TID) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 200 mg three times daily (TID) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 200 mg three times daily (TID) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 200 mg three times daily (TID) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 200 mg three times daily (TID) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 200 mg three times daily (TID) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 150 mg four times daily (QID) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 150 mg four times daily (QID) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 150 mg four times daily (QID) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 150 mg four times daily (QID) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 150 mg four times daily (QID) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 150 mg four times daily (QID) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 150 mg four times daily (QID) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 150 mg every six hours (Q6H) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 150 mg every six hours (Q6H) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 150 mg every six hours (Q6H) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 150 mg every six hours (Q6H) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 150 mg every six hours (Q6H) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 150 mg every six hours (Q6H) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 150 mg every six hours (Q6H) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 800 mg per day of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 800 mg per day of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 800 mg per day of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 800 mg per day of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 800 mg per day of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 800 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 800 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 400 mg twice daily (BID) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 400 mg twice daily (BID) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 400 mg twice daily (BID) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 400 mg twice daily (BID) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 400 mg twice daily (BID) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 400 mg twice daily (BID) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 400 mg twice daily (BID) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 900 mg per day of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 900 mg per day of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 900 mg per day of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 900 mg per day of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 900 mg per day of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 900 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 900 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 450 mg twice daily (BID) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 450 mg twice daily (BID) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 450 mg twice daily (BID) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 450 mg twice daily (BID) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 450 mg twice daily (BID) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 450 mg twice daily (BID) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 450 mg twice daily (BID) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 1000 mg per day of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 1000 mg per day of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 1000 mg per day of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 1000 mg per day of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 1000 mg per day of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 1000 mg per day of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 1000 mg per day of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is:
about 500 mg twice daily (BID) of Compound I, or a pharmaceutically acceptable salt thereof, administered for four, five, six, or seven days; and
followed thereafter by a second dose of about 200 mg per day of Compound I, or a pharmaceutically acceptable salt thereof.

In some embodiments, the first dose is:
about 500 mg twice daily (BID) of Compound I, or a pharmaceutically acceptable salt thereof, administered for four, five, six, or seven days; and
followed thereafter by a second dose of about 175 mg per day of Compound I, or a pharmaceutically acceptable salt thereof.

In some embodiments, the first dose is:
about 500 mg twice daily (BID) of Compound I, or a pharmaceutically acceptable salt thereof, administered for four, five, six, or seven days; and
followed thereafter by a second dose of about 150 mg per day of Compound I, or a pharmaceutically acceptable salt thereof.

In some embodiments, the first dose is:
about 500 mg twice daily (BID) of Compound I, or a pharmaceutically acceptable salt thereof, administered for four, five, six, or seven days; and
followed thereafter by a second dose of about 125 mg per day of Compound I, or a pharmaceutically acceptable salt thereof.

In some embodiments, the first dose is:
about 500 mg twice daily (BID) of Compound I, or a pharmaceutically acceptable salt thereof, administered for four, five, six, or seven days; and
followed thereafter by a second dose of about 100 mg per day of Compound I, or a pharmaceutically acceptable salt thereof.

In some embodiments, the first dose is:
about 500 mg twice daily (BID) of Compound I, or a pharmaceutically acceptable salt thereof, administered for four, five, six, or seven days; and
followed thereafter by a second dose of about 75 mg per day of Compound I, or a pharmaceutically acceptable salt thereof.

In some embodiments, the first dose is:
about 500 mg twice daily (BID) of Compound I, or a pharmaceutically acceptable salt thereof, administered for four, five, six, or seven days; and
followed thereafter by a second dose of about 50 mg per day of Compound I, or a pharmaceutically acceptable salt thereof.

In some embodiments, the first dose is administered for four days. In some embodiments, the first dose is administered for five days. In some embodiments, the first dose is administered for six days. In some embodiments, the first dose is administered for seven days.

In some embodiments, the first dose of Compound I, or a pharmaceutically acceptable salt thereof, is administered twice daily (BID), and the second dose of Compound I is administered once daily (QD) thereafter.

In some embodiments, the first dose of Compound I, or a pharmaceutically acceptable salt thereof, is administered three times daily (TID), and the second dose of Compound I is administered once daily (QD) thereafter.

In some embodiments, the first dose of Compound I, or a pharmaceutically acceptable salt thereof, is administered four times daily (QID), and the second dose of Compound I is administered once daily (QD) thereafter.

In some embodiments, the first dose of Compound I, or a pharmaceutically acceptable salt thereof, is administered every six hours (Q6H), and the second dose of Compound I is administered once daily (QD) thereafter.

In some embodiments, the second dose of Compound I, or a pharmaceutically acceptable salt thereof, is administered once daily (QD). In some embodiments, the second dose of Compound I, or a pharmaceutically acceptable salt thereof, is administered twice daily (BID). In some embodiments, the second dose of Compound I, or a pharmaceutically acceptable salt thereof, is administered three times daily (TID). In some embodiments, the second dose of Compound I, or a pharmaceutically acceptable salt thereof, is administered four times daily (QID). In some embodiments, the second dose of Compound I, or a pharmaceutically acceptable salt thereof, is administered every six hours (Q6H).

In some embodiments, the first dose of Compound I, or a pharmaceutically acceptable salt thereof, is administered twice daily (BID), and the second dose of Compound I, or a pharmaceutically acceptable salt thereof, is administered once daily (QD) thereafter.

In embodiments disclosed herein, the second dose is administered for as long as the patient requires or benefits from the therapeutic effect provided by such administration.

In some embodiments, the second dose is administered for one to four weeks. In some embodiments, the second dose is administered for one to five weeks. In some embodiments, the second dose is administered for one to six weeks. In some embodiments, the second dose is administered for one to seven weeks. In some embodiments, the second dose is administered for one to eight weeks. In some embodiments, the second dose is administered for one to nine weeks. In some embodiments, the second dose is administered for one to ten weeks. In some embodiments, the second dose is administered for one to twenty weeks. In some embodiments, the second dose is administered for one to thirty weeks. In some embodiments, the second dose is administered for one to forty weeks. In some embodiments, the second dose is administered for one to fifty weeks. In some embodiments, the second dose is administered for one to sixty weeks. In some embodiments, the second dose is administered for one to seventy weeks. In some embodiments, the second dose is administered for one to eighty weeks. In some embodiments, the second dose is administered for one to ninety weeks. In some embodiments, the second dose is administered for one to one hundred weeks.

In some embodiments, the second dose is administered for at least one week. In some embodiments, the second dose is administered for at least two weeks. In some embodiments, the second dose is administered for at least three weeks. In some embodiments, the second dose is administered for at least four weeks. In some embodiments, the second dose is administered for at least five weeks. In some embodiments, the second dose is administered for at least six weeks. In some embodiments, the second dose is administered for at least seven weeks. In some embodiments, the second dose is administered for at least eight weeks. In some embodiments, the second dose is administered for at least nine weeks. In some embodiments, the second dose is administered for at least ten weeks. In some embodiments, the second dose is administered for at least fifteen weeks. In some embodiments, the second dose is administered for at least twenty weeks. In some embodiments, the second dose is administered for at least thirty weeks. In some embodiments, the second dose is administered for at least forty weeks. In some embodiments, the second dose is administered for at least fifty weeks. In some embodiments, the second dose is administered for at least sixty weeks. In some embodiments, the second dose is administered for at least seventy weeks. In some embodiments, the second dose is administered for at least eight weeks. In some embodiments, the second dose is administered for at least ninety weeks. In some embodiments, the second dose is administered for at least one hundred weeks.

In some embodiments, the second dose is administered for at least one year. In some embodiments, the second dose is administered for at least two years. In some embodiments, the second dose is administered for at least three years. In some embodiments, the second dose is administered for at least four years. In some embodiments, the second dose is administered for at least five years. In some embodiments, the second dose is administered for at least six years. In some embodiments, the second dose is administered for at least seven years. In some embodiments, the second dose is administered for at least eight years. In some embodiments, the second dose is administered for at least nine years. In some embodiments, the second dose is administered for at least ten years.

In some embodiments, the second dose is administered for the lifetime of the subject. In some embodiments, the second dose is administered until the subject dies.

In some embodiments, the first dose and second dose are administered consecutively. For example, in some embodiments, the first dose is administered consecutively over four days, and the second dose is first administered on the fifth day.

In some embodiments, the percent hemoglobin occupancy in the subject, as provided by the administration of Compound I, or a pharmaceutically acceptable salt thereof, reaches about 25% to about 60% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 60% after 8 hours to about 20 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 60% after 8 hours to about 16 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 60% after 8 hours to about 12 hours following the completion of the first dose (or loading dose) of Compound I, or a pharmaceutically acceptable salt thereof.

In some embodiments, the percent hemoglobin occupancy in the subject, as provided by the administration of Compound I, or a pharmaceutically acceptable salt thereof, reaches about 25% to about 55% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 50% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 45% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 40% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 35% after about 8 hours to about 24 hours following the completion of the first dose (or loading dose) of Compound I, or a pharmaceutically acceptable salt thereof.

In some embodiments, the percent hemoglobin occupancy in the subject, as provided by the administration of Compound I, or a pharmaceutically acceptable salt thereof, reaches about 25% to about 60% following a loading dose regimen as described herein. For example, in some embodiments, the percent hemoglobin occupancy in the subject, as provided by administration of Compound I, or a pharmaceutically acceptable salt thereof, reaches about 25% to about 60% following a loading dose regimen that comprises administration of 300 mg of Compound I, or a pharmaceutically acceptable salt thereof, twice daily (BID) for 4 days.

In some embodiments, the hemoglobin occupancy in the subject, as provided by the administration of Compound I, or a pharmaceutically acceptable salt thereof, reaches about 25% to about 60% prior to administration of the second dose (or maintenance dose) of Compound I, or a pharmaceutically acceptable salt thereof. For example, in some embodiments, the percent hemoglobin occupancy in the subject, as provided by the administration of Compound I, or a pharmaceutically acceptable salt thereof, reaches about 25% to about 60% following a loading dose regimen that comprises administration of 300 mg of Compound I, or a pharmaceutically acceptable salt thereof, twice daily (BID) for 4 days, and prior to administration of the second dose (or maintenance dose) of Compound I, or a pharmaceutically acceptable salt thereof.

In some embodiments, the administration of Compound I, or a pharmaceutically acceptable salt thereof, provides a hematocrit value of about 20% to about 50% in the subject.

In some embodiment, the administration of Compound I, or a pharmaceutically acceptable salt thereof, provides a hematocrit value of at least about 20% in the subject. In some embodiment, the administration of Compound I, or a pharmaceutically acceptable salt thereof, provides a hematocrit value of at least about 30% in the subject. In some embodiment, the administration of Compound I, or a pharmaceutically acceptable salt thereof, provides a hematocrit value of about 40% in the subject. In some embodiment, the administration of Compound I, or a pharmaceutically acceptable salt thereof, provides a hematocrit value of about 45% in the subject.

In some embodiments, the administration of Compound I, or a pharmaceutically acceptable salt thereof, provides a blood concentration of Compound I of about 50 μg/mL to about 800 μg/mL. In some embodiments, the administration of Compound I, or a pharmaceutically acceptable salt thereof, provides a blood concentration of Compound I of about 95 μg/mL to about 775 μg/mL. In some embodiments, the administration of Compound I, or a pharmaceutically acceptable salt thereof, provides a blood concentration of Compound I of about 95 μg/mL to about 750 μg/mL. In some embodiments, the administration of Compound I, or a pharmaceutically acceptable salt thereof, provides a blood concentration of Compound I of about 140 μg/mL to about 700 μg/mL. In some embodiments, the administration of Compound I, or a pharmaceutically acceptable salt thereof, provides a blood concentration of Compound I of about 190 μg/mL to about 650 μg/mL. In some embodiments, the administration of Compound I, or a pharmaceutically acceptable salt thereof, provides a blood concentration of Compound I of about 250 μg/mL to about 475 μg/mL. In some embodiments, the administration of Compound I, or a pharmaceutically acceptable salt thereof, provides a blood concentration of Compound I of about 300 μg/mL to about 450 μg/mL. In some embodiments, the administration of Compound I, or a pharmaceutically acceptable salt thereof, provides a blood concentration of Compound I of about 300 μg/mL to about 475 μg/mL.

In some embodiments, the administration of Compound I, or a pharmaceutically acceptable salt thereof, provides an AUC₀₋₂₄ of Compound I of about 1,200 μg*h/mL to about 19,200 μg*h/mL. In some embodiments, the administration of Compound I, or a pharmaceutically acceptable salt thereof, provides an AUC₀₋₂₄ of Compound I of about 2,280 μg*h/mL to about 18,600 μg*h/mL. In some embodiments, the administration of Compound I, or a pharmaceutically acceptable salt thereof, provides an AUC₀₋₂₄ of Compound I of about 2,280 μg*h/mL to about 18,000 μg*h/mL. In some embodiments, the administration of Compound I, or a pharmaceutically acceptable salt thereof, provides an AUC₀₋₂₄ of Compound I of about 3,360 μg*h/mL to about 16,800 μg*h/mL. In some embodiments, the administration of Compound I, or a pharmaceutically acceptable salt thereof, provides an AUC₀₋₂₄ of Compound I of about 4,560 μg*h/mL to about 15,600 μg*h/mL. In some embodiments, the administration of Compound I, or a pharmaceutically acceptable salt thereof, provides an AUC₀₋₂₄ of Compound I of about 6,000 μg*h/mL to about 11,400 μg*h/mL. In some embodiments, the administration of Compound I, or a pharmaceutically acceptable salt thereof, provides an AUC₀₋₂₄ of Compound I of about 7,200 μg*h/mL to about 10,800 μg*h/mL. In some embodiments, the administration of Compound I, or a pharmaceutically acceptable salt thereof, provides an AUC₀₋₂₄ of Compound I of about 7,200 μg*h/mL to about 11,400 μg*h/mL.

In some embodiments, the administration of Compound I, or a pharmaceutically acceptable salt thereof, provides an AUC₀₋₂₄ of Compound I of more than about 1,200 μg*h/mL. In some embodiments, the administration of Compound I, or a pharmaceutically acceptable salt thereof, provides an AUC₀₋₂₄ of Compound I of more than about 2,280 μg*h/mL. In some embodiments, the administration of Compound I, or a pharmaceutically acceptable salt thereof, provides an AUC₀₋₂₄ of Compound I of more than about 3,360 μg*h/mL. In some embodiments, the administration of Compound I, or a pharmaceutically acceptable salt thereof, provides an AUC₀₋₂₄ of Compound I of more than about 4,560 μg*h/mL. In some embodiments, the administration of Compound I, or a pharmaceutically acceptable salt thereof, provides an AUC₀₋₂₄ of Compound I of more than about 6,000 g*h/mL. In some embodiments, the administration of Compound I, or a pharmaceutically acceptable salt thereof, provides an AUC₀₋₂₄ of Compound I of more than about 7,200 μg*h/mL. In some embodiments, the administration of Compound I, or a pharmaceutically acceptable salt thereof, provides an AUC₀₋₂₄ of Compound I of more than about 10,800 μg*h/mL. In some embodiments, the administration of Compound I, or a pharmaceutically acceptable salt thereof, provides an AUC₀₋₂₄ of Compound I of more than about 11,400 μg*h/mL. In some embodiments, the administration of Compound I, or a pharmaceutically acceptable salt thereof, provides an AUC₀₋₂₄ of Compound I of more than about 15,600 μg*h/mL. In some embodiments, the administration of Compound I, or a pharmaceutically acceptable salt thereof, provides an AUC₀₋₂₄ of Compound I of more than about 16,800 g*h/mL. In some embodiments, the administration of Compound I, or a pharmaceutically acceptable salt thereof, provides an AUC₀₋₂₄ of Compound I of more than about 18,000 μg*h/mL. In some embodiments, the administration of Compound I, or a pharmaceutically acceptable salt thereof, provides an AUC₀₋₂₄ of Compound I of more than about 18,600 μg*h/mL. In some embodiments, the administration of Compound I, or a pharmaceutically acceptable salt thereof, provides an AUC₀₋₂₄ of Compound I of more than about 19,000 μg*h/mL.

In some embodiments, Compound I, or a pharmaceutically acceptable salt thereof, is administered as a pharmaceutical composition. In some embodiments, Compound I is administered as a pharmaceutical composition.

In some embodiments, Compound I, or a pharmaceutically acceptable salt thereof, is administered orally.

Some embodiments provided herein further comprise administering an additional therapeutic agent.

In some embodiments, the additional therapeutic agent is a modulator of hemoglobin. In some embodiments, the additional therapeutic agent is useful for treating sickle cell disease. In some embodiments, the additional therapeutic agent is useful for treating a complication of sickle cell disease. Non-limiting examples of a complication of sickle cell disease include iron overload, pain, infections, acute chest syndrome, stroke, and pulmonary hypertension. In some embodiments, the additional therapeutic agent is hydroxyurea, L-glutamine, crizanlizumab, or deferiprone. In some embodiments, the additional therapeutic agent is hydroxyurea.

It is to be understood that the method of treatment embodiments of the present disclosure can be presented in a use type format.

Accordingly, the present disclosure provides a compound for use in a method of treating sickle cell disease in a subject in need thereof, wherein the compound is Compound I, or a pharmaceutically acceptable salt thereof, and wherein the method is as described herein.

Some embodiments of the present disclosure provide a compound for use in a method of treating sickle cell disease in a subject in need thereof, wherein the compound is Compound I of formula:

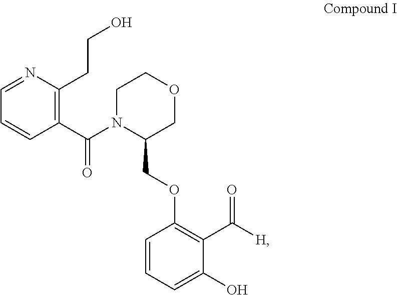

or a pharmaceutically acceptable salt thereof, and wherein the method comprises administering to the subject a first dose of Compound I, or a pharmaceutically acceptable salt thereof, for one, two, three, four, five, six, or seven days, followed thereafter by a second dose of Compound I, or a pharmaceutically acceptable salt thereof, wherein the administrations of the first dose and the second dose provide about 25% to about 60% hemoglobin occupancy by Compound I.

In some embodiments of the compound for use, the administration of the first dose and the second dose of Compound I, or a pharmaceutically acceptable salt thereof, provide about 30% to about 50% hemoglobin occupancy by Compound I, or a pharmaceutically acceptable salt thereof.

Some embodiments of the present disclosure provide a compound for use in a method of treating sickle cell disease in a subject in need thereof, wherein the compound is Compound I of formula:

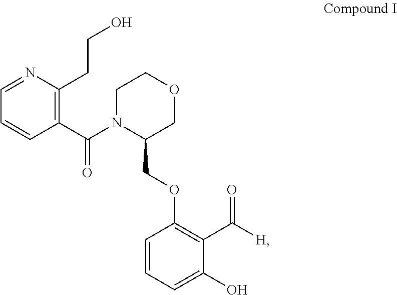

or a pharmaceutically acceptable salt thereof, and wherein the method comprises administering to the subject a first dose of Compound I, or a pharmaceutically acceptable salt thereof, for one, two, three, four, five, six, or seven days, followed thereafter by a second dose of about 25 mg per day to about 500 mg per day of Compound I, or a pharmaceutically acceptable salt thereof.

Some embodiments of the present disclosure provide a compound for use in a method of treating sickle cell disease in a subject in need thereof, wherein the compound is Compound I of formula:

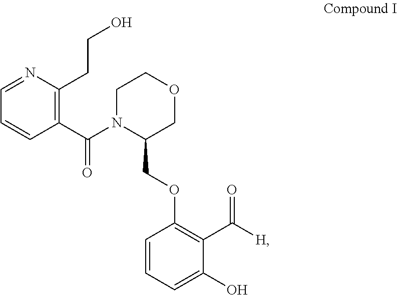

or a pharmaceutically acceptable salt thereof, and wherein the method comprises administering to the subject a first dose of about 200 mg twice daily (BID) of Compound I, or a pharmaceutically acceptable salt thereof, for four days, followed thereafter by a second dose of 100 mg per day of Compound I, or a pharmaceutically acceptable salt thereof.

Some embodiments of the present disclosure provide a compound for use in a method of treating sickle cell disease in a subject in need thereof, wherein the compound is Compound I of formula:

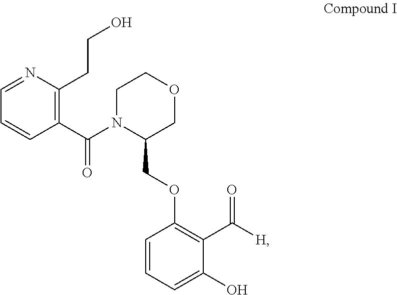

or a pharmaceutically acceptable salt thereof, and wherein the method comprises administering to the subject a first dose of about 300 mg twice daily (BID) of Compound I, or a pharmaceutically acceptable salt thereof, for four days, followed thereafter by a second dose of 150 mg per day of Compound I, or a pharmaceutically acceptable salt thereof.

Some embodiments of the present disclosure provide a compound for use in a method of treating sickle cell disease in a subject in need thereof, wherein the compound is Compound I of formula:

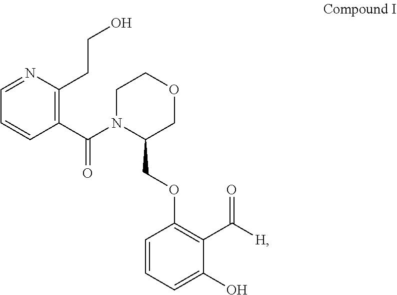

or a pharmaceutically acceptable salt thereof, and wherein the method comprises administering to the subject a first dose of about 400 mg twice daily (BID) of Compound I, or a pharmaceutically acceptable salt thereof, for four days, followed thereafter by a second dose of 200 mg per day of Compound I, or a pharmaceutically acceptable salt thereof. In some embodiments of the compound for use in a method of treating sickle cell disease, the method comprises administering to the subject a first dose of about 300 mg per day to about 1500 mg per day of Compound I, or a pharmaceutically acceptable salt thereof, for one, two, three, four, five, six, or seven days, followed thereafter by a second dose of about 25 mg per day to about 250 mg per day of Compound I, or a pharmaceutically acceptable salt thereof.

In some embodiments of the compound for use in a method of treating sickle cell disease, the method comprises administering to the subject a first dose of about 200 mg per day to about 900 mg per day of Compound I, or a pharmaceutically acceptable salt thereof, for one, two, three, four, five, six, or seven days, followed thereafter by a second dose of about 25 mg per day to about 250 mg per day of Compound I, or a pharmaceutically acceptable salt thereof.

In some embodiments of the compound for use in a method of treating sickle cell disease, the second dose is about 25 mg per day, 50 mg per day, 100 mg per day, 125 mg per day, 150 mg per day, 200 mg per day, or 250 mg per day of Compound I, or a pharmaceutically acceptable salt thereof.

In some embodiments of the compound for use in a method of treating sickle cell disease, the first dose is about 400 mg per day of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose is about 600 mg per day of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose is about 800 mg per day of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the methods described herein, or some embodiments of the compound for use in a method of treating sickle cell disease, the first dose of Compound I, or a pharmaceutically acceptable salt thereof, is administered once daily (QD), twice daily (BID), three times daily (TID), four times daily (QID), or every six hours (Q4H). In some embodiments of the methods described herein, or some embodiments of the compound for use, the first dose of Compound I, or a pharmaceutically acceptable salt thereof, is administered once daily (QD). In some embodiments of the methods described herein, or some embodiments of the compound for use, the first dose of Compound I, or a pharmaceutically acceptable salt thereof, is administered twice daily (BID). In some embodiments of the methods described herein, or some embodiments of the compound for use, the first dose of Compound I, or a pharmaceutically acceptable salt thereof, is administered three times daily (TID). In some embodiments of the methods described herein, or some embodiments of the compound for use, the first dose of Compound I, or a pharmaceutically acceptable salt thereof, is administered four times daily (QID). In some embodiments of the methods described herein, or some embodiments of the compound for use, the first dose of Compound I, or a pharmaceutically acceptable salt thereof, is administered every six hours (Q4H).

In some embodiments of the compound for use in a method of treating sickle cell disease, the first dose is about 200 mg twice daily (BID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose is about 300 mg twice daily (BID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose is about 200 mg three times daily (TID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose is about 150 mg four times daily (QID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose is about 150 mg every six hours (Q6H) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose is about 400 mg twice daily (BID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

In some embodiments of the compound for use in a method of treating sickle cell disease, the first dose of Compound I, or a pharmaceutically acceptable salt thereof, is administered for four days.

In some embodiments of the compound for use in a method of treating sickle cell disease, the second dose of Compound I, or a pharmaceutically acceptable salt thereof, is administered for at least one week, at least ten weeks, or at least fifty weeks. In some embodiments of the compound for use, the second dose of Compound I, or a pharmaceutically acceptable salt thereof, is administered for at least one week. In some embodiments of the compound for use, the second dose of Compound I, or a pharmaceutically acceptable salt thereof, is administered for at least ten weeks. In some embodiments of the compound for use, the second dose of Compound I, or a pharmaceutically acceptable salt thereof, is administered for at least fifty weeks.

In some embodiments of the compound for use in a method of treating sickle cell disease, the second dose of Compound I, or a pharmaceutically acceptable salt thereof, is administered for at least one year, at least five years, or at least ten years. In some embodiments of the compound for use, the second dose of Compound I, or a pharmaceutically acceptable salt thereof, is administered for at least one year. In some embodiments of the compound for use, the second dose of Compound I, or a pharmaceutically acceptable salt thereof, is administered for at least five years. In some embodiments of the compound for use, the second dose of Compound I, or a pharmaceutically acceptable salt thereof, is administered for at least ten years.

In some embodiments of the compound for use in a method of treating sickle cell disease, the second dose of Compound I, or a pharmaceutically acceptable salt thereof, is administered for the lifetime of the subject. In some embodiments of the compound for use, the second dose of Compound I, or a pharmaceutically acceptable salt thereof, is administered until the subject dies.

In some embodiments of the compound for use, the second dose of Compound I, or a pharmaceutically acceptable salt thereof, is administered once daily (QD).

In some embodiments of the compound for use in a method of treating sickle cell disease, the percent hemoglobin occupancy in the subject, as provided by the administration of Compound I, or a pharmaceutically acceptable salt thereof, reaches about 25% to about 60% after about 8 hours to about 24 hours following the completion of the first dose (or loading dose) of Compound I, or a pharmaceutically acceptable salt thereof. In some embodiments of the compound for use, the percent hemoglobin occupancy in the subject, as provided by the administration of Compound I, or a pharmaceutically acceptable salt thereof, reaches about 25% to about 60% after 8 hours to about 20 hours following the completion of the first dose (or loading dose) of Compound I, or a pharmaceutically acceptable salt thereof. In some embodiments of the compound for use, the percent hemoglobin occupancy in the subject, as provided by the administration of Compound I, or a pharmaceutically acceptable salt thereof, reaches about 25% to about 60% after 8 hours to about 16 hours following the completion of the first dose (or loading dose) of Compound I, or a pharmaceutically acceptable salt thereof. In some embodiments of the compound for use, the percent hemoglobin occupancy in the subject, as provided by the administration of Compound I, or a pharmaceutically acceptable salt thereof, reaches about 25% to about 60% after 8 hours to about 12 hours following the completion of the first dose (or loading dose) of Compound I, or a pharmaceutically acceptable salt thereof. In some embodiments of the compound for use, the administration of the first dose and the second dose of Compound I provide about 30% to about 50% hemoglobin occupancy by Compound I

In some embodiments of the compound for use in a method of treating sickle cell disease, the percent hemoglobin occupancy in the subject, as provided by the administration of Compound I, or a pharmaceutically acceptable salt thereof, reaches about 25% to about 60% following a loading dose regimen as described herein. For example, in some embodiments of the compound for use, the percent hemoglobin occupancy in the subject, as provided by administration of Compound I, or a pharmaceutically acceptable salt thereof, reaches about 25% to about 60% following a loading dose regimen that comprises administration of 300 mg of Compound I, or a pharmaceutically acceptable salt thereof, twice daily (BID) for 4 days.

In some embodiments of the compound for use in a method of treating sickle cell disease, the percent hemoglobin occupancy in the subject, as provided by the administration of Compound I, or a pharmaceutically acceptable salt thereof, reaches about 25% to about 60% prior to administration of the second dose (or maintenance dose) of Compound I, or a pharmaceutically acceptable salt thereof. For example, in some embodiments of the compound for use, the percent hemoglobin occupancy in the subject, as provided by the administration of Compound I, or a pharmaceutically acceptable salt thereof, reaches about 25% to about 60% following a loading dose regimen that comprises administration of 300 mg of Compound I, or a pharmaceutically acceptable salt thereof, twice daily (BID) for 4 days, and prior to administration of the second dose (or maintenance dose) of Compound I, or a pharmaceutically acceptable salt thereof.

In some embodiments of the compound for use in a method of treating sickle cell disease, the administration of Compound I, or a pharmaceutically acceptable salt thereof, provides a hematocrit value of about 20% to about 50% in the subject.

In some embodiments of the compound for use in a method of treating sickle cell disease, the administration of Compound I, or a pharmaceutically acceptable salt thereof, provides a hematocrit value of at least about 20% in the subject. In some embodiment, the administration of Compound I, or a pharmaceutically acceptable salt thereof, provides a hematocrit value of at least about 30% in the subject. In some embodiment of the compound for use, the administration of Compound I, or a pharmaceutically acceptable salt thereof, provides a hematocrit value of about 40% in the subject. In some embodiment, the administration of Compound I, or a pharmaceutically acceptable salt thereof, provides a hematocrit value of about 45% in the subject.

In some embodiments of the compound for use in a method of treating sickle cell disease, the administration of Compound I, or a pharmaceutically acceptable salt thereof, provides a blood concentration of Compound I of about 50 μg/mL to about 800 μg/mL. In some embodiments of the compound for use in a method of treating sickle cell disease, the administration of Compound I, or a pharmaceutically acceptable salt thereof, provides a blood concentration of Compound I of about 95 g/mL to about 775 μg/mL. In some embodiments of the compound for use in a method of treating sickle cell disease, the administration of Compound I, or a pharmaceutically acceptable salt thereof, provides a blood concentration of Compound I of about 95 μg/mL to about 750 μg/mL. In some embodiments of the compound for use in a method of treating sickle cell disease, the administration of Compound I, or a pharmaceutically acceptable salt thereof, provides a blood concentration of Compound I of about 140 μg/mL to about 700 μg/mL. In some embodiments of the compound for use in a method of treating sickle cell disease, the administration of Compound I, or a pharmaceutically acceptable salt thereof, provides a blood concentration of Compound I of about 190 μg/mL to about 650 μg/mL. In some embodiments of the compound for use in a method of treating sickle cell disease, the administration of Compound I, or a pharmaceutically acceptable salt thereof, provides a blood concentration of Compound I of about 250 μg/mL to about 475 μg/mL. In some embodiments of the compound for use in a method of treating sickle cell disease, the administration of Compound I, or a pharmaceutically acceptable salt thereof, provides a blood concentration of Compound I of about 300 μg/mL to about 450 μg/mL. In some embodiments of the compound for use in a method of treating sickle cell disease, the administration of Compound I, or a pharmaceutically acceptable salt thereof, provides a blood concentration of Compound I of about 300 μg/mL to about 475 μg/mL.

In some embodiments of the compound for use in a method of treating sickle cell disease, the administration of Compound I, or a pharmaceutically acceptable salt thereof, provides an AUC₀₋₂₄ of Compound I of about 1,200 μg*h/mL to about 19,200 μg*h/mL. In some embodiments of the compound for use in a method of treating sickle cell disease, the administration of Compound I, or a pharmaceutically acceptable salt thereof, provides an AUC₀₋₂₄ of Compound I of about 2,280 μg*h/mL to about 18,600 μg*h/mL. In some embodiments of the compound for use in a method of treating sickle cell disease, the administration of Compound I, or a pharmaceutically acceptable salt thereof, provides an AUC₀₋₂₄ of Compound I of about 2,280 μg*h/mL to about 18,000 μg*h/mL. In some embodiments of the compound for use in a method of treating sickle cell disease, the administration of Compound I, or a pharmaceutically acceptable salt thereof, provides an AUC₀₋₂₄ of Compound I of about 3,360 μg*h/mL to about 16,800 μg*h/mL. In some embodiments of the compound for use in a method of treating sickle cell disease, the administration of Compound I, or a pharmaceutically acceptable salt thereof, provides an AUC₀₋₂₄ of Compound I of about 4,560 μg*h/mL to about 15,600 μg*h/mL. In some embodiments of the compound for use in a method of treating sickle cell disease, the administration of Compound I, or a pharmaceutically acceptable salt thereof, provides an AUC₀₋₂₄ of Compound I of about 6,000 μg*h/mL to about 11,400 μg*h/mL. In some embodiments of the compound for use in a method of treating sickle cell disease, the administration of Compound I, or a pharmaceutically acceptable salt thereof, provides an AUC₀₋₂₄ of Compound I of about 7,200 μg*h/mL to about 10,800 μg*h/mL. In some embodiments of the compound for use in a method of treating sickle cell disease, the administration of Compound I, or a pharmaceutically acceptable salt thereof, provides an AUC₀₋₂₄ of Compound I of about 7,200 μg*h/mL to about 11,400 μg*h/mL.

In some embodiments of the compound for use in a method of treating sickle cell disease, the administration of Compound I, or a pharmaceutically acceptable salt thereof, provides an AUC₀₋₂₄ of Compound I of more than about 1,200 μg*h/mL. In some embodiments of the compound for use in a method of treating sickle cell disease, the administration of Compound I, or a pharmaceutically acceptable salt thereof, provides an AUC₀₋₂₄ of Compound I of more than about 2,280 μg*h/mL. In some embodiments of the compound for use in a method of treating sickle cell disease, the administration of Compound I, or a pharmaceutically acceptable salt thereof, provides an AUC₀₋₂₄ of Compound I of more than about 3,360 μg*h/mL. In some embodiments of the compound for use in a method of treating sickle cell disease, the administration of Compound I, or a pharmaceutically acceptable salt thereof, provides an AUC₀₋₂₄ of Compound I of more than about 4,560 μg*h/mL. In some embodiments of the compound for use in a method of treating sickle cell disease, the administration of Compound I, or a pharmaceutically acceptable salt thereof, provides an AUC₀₋₂₄ of Compound I of more than about 6,000 μg*h/mL. In some embodiments of the compound for use in a method of treating sickle cell disease, the administration of Compound I, or a pharmaceutically acceptable salt thereof, provides an AUC₀₋₂₄ of Compound I of more than about 7,200 μg*h/mL. In some embodiments of the compound for use in a method of treating sickle cell disease, the administration of Compound I, or a pharmaceutically acceptable salt thereof, provides an AUC₀₋₂₄ of Compound I of more than about 10,800 μg*h/mL. In some embodiments of the compound for use in a method of treating sickle cell disease, the administration of Compound I, or a pharmaceutically acceptable salt thereof, provides an AUC₀₋₂₄ of Compound I of more than about 11,400 μg*h/mL. In some embodiments of the compound for use in a method of treating sickle cell disease, the administration of Compound I, or a pharmaceutically acceptable salt thereof, provides an AUC₀₋₂₄ of Compound I of more than about 15,600 μg*h/mL. In some embodiments of the compound for use in a method of treating sickle cell disease, the administration of Compound I, or a pharmaceutically acceptable salt thereof, provides an AUC₀₋₂₄ of Compound I of more than about 16,800 μg*h/mL. In some embodiments of the compound for use in a method of treating sickle cell disease, the administration of Compound I, or a pharmaceutically acceptable salt thereof, provides an AUC₀₋₂₄ of Compound I of more than about 18,000 g*h/mL. In some embodiments of the compound for use in a method of treating sickle cell disease, the administration of Compound I, or a pharmaceutically acceptable salt thereof, provides an AUC₀₋₂₄ of Compound I of more than about 18,600 μg*h/mL. In some embodiments of the compound for use in a method of treating sickle cell disease, the administration of Compound I, or a pharmaceutically acceptable salt thereof, provides an AUC₀₋₂₄ of Compound I of more than about 19,000 μg*h/mL.

In some embodiments of the compound for use in a method of treating sickle cell disease, the first dose of Compound I, or a pharmaceutically acceptable salt thereof, is administered as a pharmaceutical composition. In some embodiments of the compound for use in a method of treating sickle cell disease, the second dose of Compound I, or a pharmaceutically acceptable salt thereof, is administered as a pharmaceutical composition.

In some embodiments of the compound for use in a method of treating sickle cell disease, Compound I, or a pharmaceutically acceptable salt thereof, is administered orally.

In some embodiments of the compound for use in a method of treating sickle cell disease, Compound I is a crystalline form characterized by an X-ray powder diffractogram comprising the following peaks: 18.3°, 23.4°, and 26.1 °2θ±0.2 °2θ (Compound I Form I), as determined on a diffractometer using Cu-Kα radiation.

In some embodiments of the compound for use in a method of treating sickle cell disease, the diffractogram further comprises one or more peaks at: 10.8° or 17.3 °2θ±0.2 °2θ.

In some embodiments of the compound for use in a method of treating sickle cell disease, the crystalline form is characterized by a differential scanning calorimetry (DSC) curve comprising an endotherm at about 111° C. (onset temperature).

In some embodiments of the compound for use in a method of treating sickle cell disease, the use in treating sickle cell disease further comprises administering an additional therapeutic agent.

In some embodiments of the compound for use in a method of treating sickle cell disease, the additional therapeutic agent is a modulator of hemoglobin. In some embodiments, the additional therapeutic agent is useful for treating sickle cell disease. In some embodiments, the additional therapeutic agent is useful for treating a complication of sickle cell disease. Non-limiting examples of a complication of sickle cell disease include iron overload, pain, infections, acute chest syndrome, stroke, and pulmonary hypertension. In some embodiments, the additional therapeutic agent is hydroxyurea, L-glutamine, crizanlizumab, or deferiprone. In some embodiments, the additional therapeutic agent is hydroxyurea.

In some embodiments of the compound for use in a method of treating sickle cell disease, the additional therapeutic agent is hydroxyurea.

### 3. Pharmaceutical Compositions and Modes of Administration

Compound I, or a pharmaceutically acceptable salt thereof, or a crystalline form thereof, as described herein may be administered in a pharmaceutical composition. Thus, provided herein are pharmaceutical compositions comprising one or more of the forms of Compound I described herein, or salts or solvates thereof, and one or more pharmaceutically acceptable vehicles such as carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17^{th} Ed. (1985); and Modern Pharmaceutics, Marcel Dekker, Inc. 3^{rd} Ed. (G. S. Banker & C. T. Rhodes, Eds.).

The pharmaceutical compositions may be administered alone or in combination with other therapeutic agents.

The pharmaceutical compositions may be administered in either single or multiple first and/or second doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein, solid forms described herein, or salts or solvates thereof. Administration may be via, for example, capsule or enteric coated tablets. In making the pharmaceutical compositions that include at least one compound described herein, solid form described herein, or salt or solvate thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active ingredient, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound or solid form described herein, or salts or solvates thereof, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds or solid forms described herein, or salts or solvates thereof, in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on-demand delivery of pharmaceutical agents.

For preparing solid compositions such as tablets, the principal active ingredient may be mixed with a pharmaceutical excipient to form a solid preformulation composition containing a homogeneous mixture of a compound or solid form described herein, or salts or solvates thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds or solid forms described herein, or salts or solvates thereof, may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, acetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device, or the nebulizing device may be attached to a facemask tent, or intermittent positive pressure breathing machine. Solution, suspension, or powder compositions may be administered, preferably orally or nasally, from devices that deliver the formulation in an appropriate manner.

### EXAMPLES

### Example 1: Healthy Volunteers and Single Ascending Dose (SAD) Study

To evaluate the safety and tolerability of single ascending doses of Compound I, 48 healthy volunteers (42 in the fasted state and 6 in the fed state) aged 18 to 55 years old were administered single ascending doses (50 mg to 2200 mg) of Compound I, while 16 healthy volunteers (14 in the fasted state and 2 in the fed state) aged 18 to 55 years old were administered placebo in a double blind, randomized, sequential manner.

Primary endpoints were safety and tolerability; key secondary endpoints included PK and effect of food (high-fat meal).

Demographics and baseline characteristics of the healthy volunteers in the blinded study are summarized in Table 1 and were similar across groups; details of the same but unblinded study are included in Table 2. All groups in the blinded study consisted of 6 healthy volunteers who received Compound I and 2 healthy volunteers who received placebo.

TABLE 1
Demographics of the Subjects in Single Ascending Dose and Blinded Study

| | | | | | | | 200 mg | | | | | | | | | |
|---|---|---|---|---|---|---|---|---|---|---|---|---|---|---|---|---|
| | 50 mg | | 100 mg | | 200 mg | | (fed) | | 400 mg | | 800 mg | | 1400 mg | | 2200 mg | |
| | (n = 8) | | (n = 8) | | (n = 8) | | (n = 8)* | | (n = 8) | | (n = 8) | | (n = 8) | | (n = 8) | |
| Mean age, | 33.3 | (5.1) | 28.5 | (10.5) | 26.5 | (9.6) | 26.5 | (9.8) | 34.4 | (11.3) | 34.1 | (10.9) | 29.6 | (8.0) | 25.4 | (4.7) |
| years (SD) | | | | | | | | | | | | | | | | |
| Male, n (%) | 8 | (100) | 7 | (87.5) | 8 | (100) | 8 | (100) | 6 | (75.0) | 7 | (87.5) | 7 | (87.5) | 8 | (100) |

Race, n (%)

| White | 4 | (50.0) | 5 | (62.5) | 6 | (75.0) | 6 | (75.0) | 2 | (25.0) | 6 | (75.0) | 7 | (87.5) | 5 | (62.5) |
|---|---|---|---|---|---|---|---|---|---|---|---|---|---|---|---|---|
| Asian | 3 | (37.5) | 2 | (25.0) | 1 | (12.5) | 2 | (25.0) | 5 | (62.5) | 0 | | 1 | (12.5) | 1 | (12.5) |
| Black or | 0 | | 0 | | 0 | | 0 | | 0 | | 1 | (12.5) | 0 | | 0 | |
| African | | | | | | | | | | | | | | | | |
| American | | | | | | | | | | | | | | | | |

| | | | | | | | | | | | | | | |
|---|---|---|---|---|---|---|---|---|---|---|---|---|---|---|
| Other | 1 | (12.5) | 1 | (12.5) | 1 | (12.5) | 0 | 1 | (12.5) | 1 | (12.5) | 0 | 2 | (25.0) |

*To determine food effect, 200 mg dose was given in fasting state and after a high-fat meal.

TABLE 2
Demographics of the Subjects in Single Ascending Dose and Unblinded Study

| | | | | | | | 200 mg | | | | | | | |
|---|---|---|---|---|---|---|---|---|---|---|---|---|---|---|
| | 50 mg | | 100 mg | | 200 mg | | (fed) | | 400 mg | | 800 mg | | 1400 mg | |
| | (n = 6) | | (n = 6) | | (n = 6) | | (n = 6)* | | (n = 6) | | (n = 6) | | (n = 6) | |
| Mean age, | 34.0 | (5.69) | 28.0 | (10.66) | 24.2 | (8.95) | 26.5 | (11.36) | 35.5 | (12.94) | 30.7 | (8.89) | 27.2 | (7.73) |
| years (SD) | | | | | | | | | | | | | | |
| Male, n (%) | 6 | (100.0) | 5 | (83.3) | 6 | (100.0) | 6 | (100.0) | 4 | (66.7) | 5 | (83.3) | 6 | (100.0) |

Race, n (%)

| White | 3 | (50.0) | 3 | (50.0) | 4 | (66.7) | 4 | (66.7) | 2 | (33.3) | 6 | (75.0) | 7 | (87.5) |
|---|---|---|---|---|---|---|---|---|---|---|---|---|---|---|
| Asian | 3 | (50.5) | 2 | (33.0) | 1 | (16.7) | 2 | (33.3) | 4 | (66.7) | 0 | | 1 | (12.5) |
| Black or | 0 | | 0 | | 1 | (16.7) | 0 | | 0 | | 1 | (12.5) | 0 | |
| African | | | | | | | | | | | | | | |
| American | | | | | | | | | | | | | | |

| | | | | | | | | | | | | |
|---|---|---|---|---|---|---|---|---|---|---|---|---|
| Other | 1 | (12.5) | 1 | (16.7) | 1 | (12.5) | 0 | 1 | (12.5) | 1 | (12.5) | 0 |

Pooled

| | |
|---|---|
| Placebo | Placebo |

| | | |
|---|---|---|
| 2200 mg | (Fasted, | (Fed, |
| (n = 6) | n = 14) | n = 2) |

| Mean age, | 24.8 | (5.12) | 33.4 | (8.36) | 26.5 | (4.95) |
|---|---|---|---|---|---|---|
| years (SD) | | | | | | |
| Male, n (%) | 6 | (100.0) | 13 | (92.9) | 2 | (100.0) |
| Race, n (%) | | | | | | |
| White | 5 | (62.5) | 10 | (7.14) | 2 | (100.0) |
| Asian | 1 | (12.5) | 0 | | 0 | |
| Black or | 0 | | 0 | | 0 | |
| African | | | | | | |
| American | | | | | | |

| | | | | | |
|---|---|---|---|---|---|
| Other | 2 | (25.0) | 3 | (21.4) | 0 |

*To determine food effect, 200 mg dose was given in fasting state and after a high-fat meal.

Adverse events that occurred following treatment in the blinded study were mostly grade 1 or 2 using the Common Terminology Criteria for Adverse Events (CTCAE) v5.0, as shown in Table 3; adverse events from the same but unblinded study are shown in Table 4. The few that were considered drug-related resolved with little to no intervention. There were no adverse events indicative of tissue hypoxia.

TABLE 3
Safety Profile in the Single Ascending Dose and Blinded Study

| | | | | 200 mg | | | | |
|---|---|---|---|---|---|---|---|---|
| Overview of | 50 mg | 100 mg | 200 mg | (fed)^{b} | 400 mg | 800 mg | 1400 mg | 2200 mg |
| TEAEs^{a} | (n = 8) | (n = 8) | (n = 8) | (n = 8) | (n = 8) | (n = 8) | (n = 8) | (n = 8) |
| Number of | 14 | 13 | 16 | 8 | 23 | 14 | 29 | 17 |
| TEAEs | | | | | | | | |
| Number of | 7 | 6 | 7 | 6 | 8 | 6 | 8 | 6 |
| participants with | (87.5) | (75.0) | (87.5) | (75.0) | (100) | (75.0) | (100) | (75.0) |
| at least 1 TEAE, | | | | | | | | |
| n (%) | | | | | | | | |
| Number of | 0 | 0 | 0 | 1 | 1 | 0 | 0 | 0 |
| TEAEs grade ≥ 3 | | | | | | | | |
| Number of | 2 | 1 | 0 | 0 | 1 | 3 | 4 | 4 |
| drug-related | | | | | | | | |
| TEAEs | | | | | | | | |
| Number of | 2 | 1 | 0 | 0 | 1 | 2 | 4 | 2 |
| participants with | (25.0) | (12.5) | | | (12.5) | (25.0) | (50.0) | (25.0) |
| at least 1 drug- | | | | | | | | |
| related TEAE, n | | | | | | | | |
| (%) | | | | | | | | |
| Number of | 0 | 0 | 0 | 0 | 0 | 0 | 0 | 0 |
| TEAEs leading | | | | | | | | |
| to study drug | | | | | | | | |
| discontinuation | | | | | | | | |
| Number of | 0 | 0 | 0 | 0 | 1^{c} | 0 | 0 | 0 |
| SAEs | | | | | | | | |

^{a}Data presented are still blinded.
^{b}To determine food effect, 200 mg dose was given in fasting state and after a high-fat meal.
^{c}The 1 SAE was a traumatic brain injury and was not drug related.
SAE, serious adverse event;
TEAE, treatment-emergent adverse event.

TABLE 4
Safety Profile in the Single Ascending Dose and Unblinded Study

| Over- | Compound I Dose (mg) | | | | | | | | Pooled | |
|---|---|---|---|---|---|---|---|---|---|---|
| view | | | | 200 mg | | | | | Placebo | |
| of | 50 mg | 100 mg | 200 mg | (n = 6 | 400 mg | 800 mg | 1400 mg | 2200 mg | Fasted | Fed |
| TEAEs^{a} | (n = 6) | (n = 6) | (n = 6) | fed)^{b} | (n = 6) | (n = 6) | (n = 6) | (n = 6) | (n = 14) | (n = 2) |
| Num- | 11 | 10 | 15 | 7 | 16 | 10 | 26 | 18 | 28 | 1 |
| ber of | | | | | | | | | | |
| TEAEs | | | | | | | | | | |
| Num- | 5 | 5 | 6 | 5 | 6 | 5 | 6 | 5 | 10 | 1 |
| ber of | (83.3) | (83.3 | (100) | (83.3) | (100) | (83.3) | (100) | (83.3) | (71.4) | (50) |
| partic- | | | | | | | | | | |
| ipants | | | | | | | | | | |
| with | | | | | | | | | | |
| at | | | | | | | | | | |
| least 1 | | | | | | | | | | |
| TEAE, | | | | | | | | | | |
| n (%) | | | | | | | | | | |
| Num- | 0 | 0 | 0 | 1 | 1 | 0 | 0 | 0 | 0 | 0 |
| ber of | | | | | | | | | | |
| TEAEs | | | | | | | | | | |
| grade ≥ | | | | | | | | | | |
| 3 | | | | | | | | | | |
| Num- | 1 | 1 | 0 | 0 | 1 | 3 | 4 | 4 | 1 | 0 |
| ber of | | | | | | | | | | |
| drug- | | | | | | | | | | |
| related | | | | | | | | | | |
| TEAEs | | | | | | | | | | |
| Num- | 1 | 1 | 0 | 0 | 1 | 2 | 4 | 2 | 1 | 0 |
| ber of | (16/7) | (16/7) | | | (16.7) | (33.3) | (66.7) | (33.3) | (7.1) | |
| partic- | | | | | | | | | | |
| ipants | | | | | | | | | | |
| with | | | | | | | | | | |
| at | | | | | | | | | | |
| least 1 | | | | | | | | | | |
| drug- | | | | | | | | | | |
| related | | | | | | | | | | |
| TEAE, | | | | | | | | | | |
| n (%) | | | | | | | | | | |
| Num- | 0 | 0 | 0 | 0 | 0 | 0 | 0 | 0 | 0 | 0 |
| ber of | | | | | | | | | | |
| TEAEs | | | | | | | | | | |
| leading | | | | | | | | | | |
| to study | | | | | | | | | | |
| drug | | | | | | | | | | |
| discon- | | | | | | | | | | |
| tinua- | | | | | | | | | | |
| tion | | | | | | | | | | |
| Num- | 0 | 0 | 0 | 0 | 1^{c} | 0 | 0 | 0 | 0 | 0 |
| ber of | | | | | | | | | | |
| SAEs | | | | | | | | | | |

^{a}Data presented are unblinded.
^{b}To determine food effect, 200 mg dose was given in fasting state and after a high-fat meal.
^{c}The 1 SAE was a traumatic brain injury and was not drug related.
SAE, serious adverse event;
TEAE, treatment-emergent adverse event.

Whole blood pharmacokinetic (PK) parameters, using actual sample collection times, and hemoglobin occupancy after single ascending dose are shown in Table 5 (containing data from both healthy volunteers and SCD subjects) and Table 6 (containing data from only healthy volunteer subjects).

TABLE 5
Whole Blood PK Parameters and Hemoglobin Occupancy after
Doses of Compound I in Both Healthy Volunteers and Subjects with SCD

| | HV | HV | SCD | HV | HV | HV | HV | HV |
|---|---|---|---|---|---|---|---|---|
| Parameter, | 50 | 100 | 100 | 200 | 400 | 800 | 1400 | 2200 |
| mean | mg | mg | mg | mg | mg | mg^{a} | mg^{a} | mg^{b} |
| t_{1/2}, days | 28.9 | 29.8 | 9.8 | 28.4 | 25.8 | ND | ND | ND |
| (CV %) | (8.4) | (22.3) | (14.7) | (15.6) | (10.8) | | | |
| Cₘₐₓ, μg/mL | 8.1 | 17.4 | 16.7 | 35.8 | 88.1 | 149.3 | 231.2 | 389.7 |
| (CV %) | (16.9) | (10.8) | (23.3) | (32.4) | (11.1) | (10.5) | (12.6) | (12.0) |
| AUC₀₋ₜ, | 6896 | 14,400 | 4690 | 29,290 | 61,920 | 74,330 | 136,900 | 48,010 |
| μg · h/mL | (21.5) | (14.7) | (26.8) | (26.1) | (9.3) | (4.5) | (7.8) | (10.0) |
| (CV %) | | | | | | | | |
| tₘₐₓ,^{c} h | 24 | 24 | 8 | 8 | 8 | 16 | 12 | 24 |
| (range) | (4, 48) | (8, 48) | (6, 36) | (4, 48) | (4, 120) | (1, 96) | (8, 48) | (12, 24) |
| B:P ratio^{d} | 220 | 214 | 69.3 | 206 | 185 | ND | ND | ND |
| % Hb | 0.88 | 1.9 | 3.3 | 3.8 | 9.5 | 16.8 | 25 | 43.9 |
| occupancy | (16.7) | (13.3) | (26.1) | (33.0) | (22.6) | (10.4) | (12.4) | (12.3) |
| based on | | | | | | | | |
| Cₘₐₓ | | | | | | | | |
| (CV %) | | | | | | | | |

^{a}Blood collected up to day 42.
^{b}Blood collected up to day 7.
^{c}tₘₐₓ reported as median.
^{d}B:P = mean AUC_{0-t blood}:mean AUC_{0-t plasma}.
AUC₀₋ₜ, area under the curve to last measurable concentration time;
Cₘₐₓ, maximal observed concentration;
CV, coefficient of variation;
Hb, hemoglobin;
HV, healthy volunteer;
ND, not determined;
PK, pharmacokinetics;
SCD, sickle cell disease;
t_{1/2}, half-life;
tₘₐₓ, time of maximal observed concentration.

TABLE 6
Geometric Mean (CV %) Values for PK Parameters After Single Oral Doses of
Compound I in Plasma, Whole Blood, and Red Blood Cells in Healthy Subjects

| | | | | | | |
|---|---|---|---|---|---|---|
| | Dose | Tₘₐₓ* | Cₘₐₓ | AUCₗₐₛₜ | AUC_{inf} | t_{1/2} |
| Cohort | (mg) | (hr) | (μg/mL) | (hr*μg/mL) | (hr*μg/mL) | (hr) |

Plasma

| | | | | | | | | | | | |
|---|---|---|---|---|---|---|---|---|---|---|---|
| 1 | 50 | 0.75 | (0.5, 1.0) | 0.40 | (15.8) | 31.1 | (17.1) | 34.2 | (19.0) | 689 | (10.2.1) |
| 2 | 100 | 1.0 | (0.5, 3.0) | 0.714 | (22.6) | 68.7 | (17.4) | 74.5 | (19.3) | 615 | (22.5) |
| 3 | 200 | 1.0 | (0.5, 2.0) | 1.29 | (23.3) | 135 | (31.0) | 152 | (34.8) | 740 | (20.4) |
| 5 | 400 | 1.0 | (0.5, 2.0) | 3.19 | (26) | 331 | (11.9) | 361 | (10.2) | 624 | (12.2) |
| 6 | 800 | 2.5 | (1.0, 3.0) | 4.25 | (22.2) | 609 | (14.6) | 623 | (12.6) | 497 | (11.8) |
| 7 | 1400 | 2.5 | (2.0, 4.0) | 7.78 | (24.3) | 1030 | (12.6) | 1080 | (12.9) | 514 | (10.6) |
| 8 | 2200 | 3.5 | (3.0, 6.0) | 9.30 | (9.7) | 1360 | (8.6) | 1430 | (9.7) | 531 | (18.7) |

Whole Blood

| | | | | | | | | | | | |
|---|---|---|---|---|---|---|---|---|---|---|---|
| 1 | 50 | 24.0 | (4, 48.02) | 8.00 | (17.3) | 6810 | (21.6) | 7490 | (23.6) | 683 | (9.6) |
| 2 | 100 | 24.0 | (8, 48) | 17.3 | (12.3) | 14300 | (14.6) | 15900 | (19.2) | 698 | (22.9) |
| 3 | 200 | 8.0 | (4.0, 48.05) | 34.2 | (33.9) | 28600 | (28.3) | 31600 | (31.0) | 677 | (14.8) |
| 5 | 400 | 8.0 | (4, 120) | 87.6 | (11.2) | 62200 | (8.8) | 67200 | (7.6) | 609 | (12.7) |
| 6 | 800 | 16.0 | (1, 96.02) | 149 | (10.2) | 103000 | (5.5) | 111000 | (7.2) | 625 | (14.4) |
| 7 | 1400 | 12.0 | (8, 48) | 230 | (12.8) | 192000 | (10.1) | 210000 | (11.9) | 656 | (11.9) |
| 8 | 2200 | 24.0 | (12, 24.15) | 387 | (12.2) | 264000 | (13.6) | 282000 | (16.1) | 590 | (15.4) |

Red Blood Cells*

| | | | | | | | | | | | |
|---|---|---|---|---|---|---|---|---|---|---|---|
| 1 | 50 | 24.0 | (4.0, 48.02) | 19.3 | (15.5) | 16500 | (20.4) | 18100 | (22.3) | 679 | (9.1) |
| 2 | 100 | 24.0 | (8.5, 48.0 | 41.7 | (23.3) | 34500 | (13.5) | 38500 | (14.0) | 696 | (23.4) |
| 3 | 200 | 8.0 | (4, 48.05) | 76.9 | (32.6) | 64400 | (25.2) | 71200 | (27.9) | 674 | (14.9) |
| 5 | 400 | 10.02 | ((4, 120) | 210 | (16.2) | 151000 | (12.7) | 163000 | (13.0) | 605 | (13.2) |
| 6 | 800 | 36.0 | (1, 144) | 351 | (16.1) | 247000 | (6.2) | 266000 | (4.9) | 623 | (14.5) |
| 7 | 1400 | 30.0 | (8, 144) | 543 | (15.4) | 455000 | (8.7) | 499000 | (9.9) | 657 | (11.9) |
| 8 | 2200 | 24.0 | (12, 24.15) | 910 | (8.8) | 626000 | (11.2) | 670000 | (13.7) | 590 | (15.4) |

AUCₗₐₛₜ, area under the concentration time curve to the last measurable concentration,
AUC_{inf}, area under the concentration time curve to infinity,
Cₘₐₓ, maximal observed concentration;
CV = coefficient of variation;
t_{1/2}, half-life;
tₘₐₓ, time of maximal observed concentration [median (range)]
*RBC concentration of Compound I = blood conc.- [(1-hematocrit)
*plasma conc.])/hematocrit

The mean blood to plasma ratios for Cₘₐₓ and AUC_{inf} ranged from 20 to 42 and 177 to 219, respectively, for the dose range of 50 to 2200 mg. In contrast, the mean RBC to plasma ratios for Cₘₐₓ and AUC_{inf} ranged from 49 to 99 and 420 to 530, respectively, for the dose range of 50 to 2200 mg.

Percent hemoglobin occupancy was calculated as follows, wherein MCHC refers to mean corpuscular hemoglobin concentration, and Hct refers to hematocrit:

$$\begin{matrix}\% Hb occupancy = {[ Compound I ]}_{RBC} / MCHC \\ {[ Compound I ]}_{RBC} = ( {[ Compound I ]}_{whole blood} - {( 1 - Hct ) [ Compound I ]}_{plasma} ) / Hct\end{matrix}$$

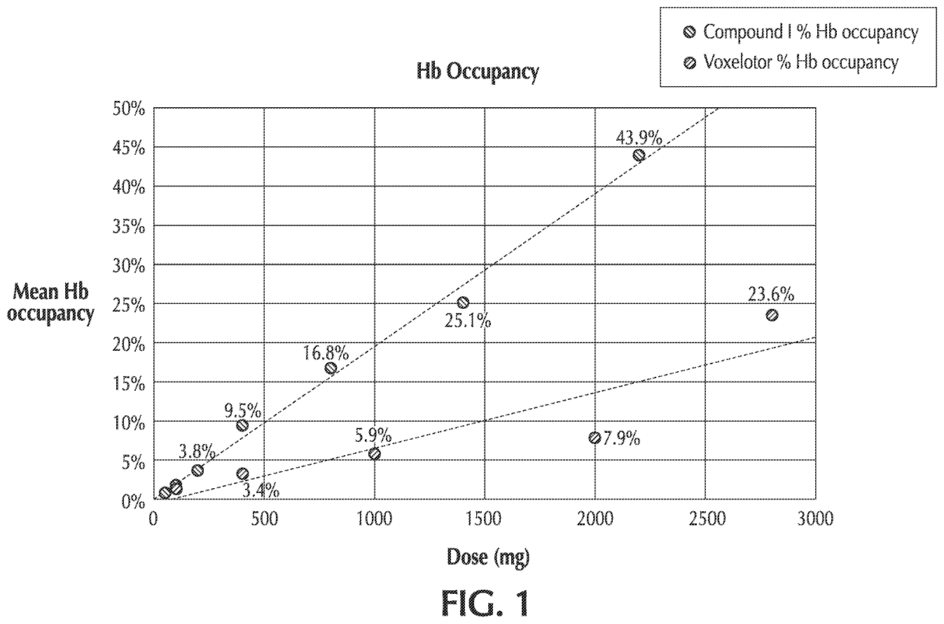

Single doses of Compound I showed a dose-dependent increase in percent Hb occupancy in healthy volunteers (FIG. 1). There was no observable difference in Hb occupancy between fasting and fed states (200 mg). These Hb occupancies exceed those reported in healthy volunteers receiving single doses of voxelotor over a similar range.

### Example 2: Healthy Volunteers and Multiple Ascending Dose (MAD)

To evaluate the safety and tolerability of multiple ascending doses (MAD) of Compound I, seven healthy volunteers aged 18 to 55 years old were administered a loading dose of 300 mg of Compound I for 3 days, followed by maintenance doses of 15 mg, 25 mg, 50 mg, or 75 mg of Compound I for 14 days. Three healthy volunteers aged 18 to 55 years old were administered placebo for 14 days. Primary endpoints were safety and tolerability; key secondary endpoints included PK and effect on electrocardiogram (ECG) parameters.

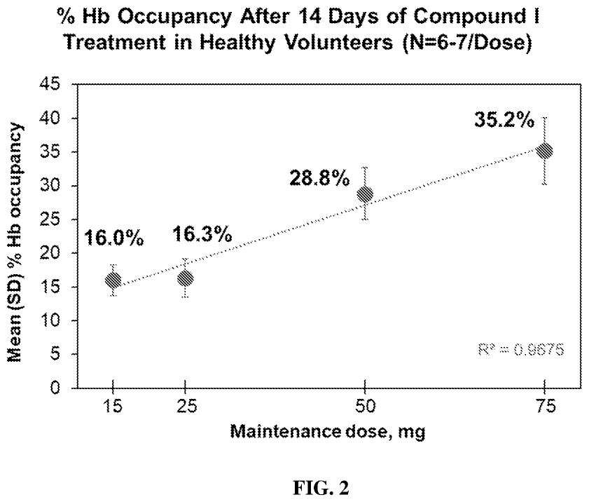

As shown in FIG. 2, Compound I demonstrated linear, dose-proportional PK among cohorts with multiple ascending doses in healthy volunteers.

Another study was initiated in healthy volunteer aged 18-55 years old to evaluate the safety and tolerability of multiple ascending doses of Compound I. Four cohorts of 10 subjects each (n=7 Compound I, n=3 placebo) were evaluated in a sequential double-blind manner. To reach steady state levels each subject received a loading dose regimen for 3 or 4 days followed by once daily medication for up to 14 days. Primary endpoints were safety and tolerability; key secondary endpoints included PK and effect on electrocardiogram (ECG) parameters. Table 7 below provides an overview of the doses of Compound I or placebo that were used in each cohort.

TABLE 7
Dosing Regimens Evaluated in Healthy
Subjects MAD Study (QD, mg/day)

| Cohort | Day 1 | Day 2 | Day 3 | Day 4 | Days 5-14 |
|---|---|---|---|---|---|
| 1^{a} | 200 | 200 | 200 | 15 | 15 |
| 2^{a} | 200 | 200 | 200 | 25 | 25 |
| 3^{a} | 500 | 400 | 400 | 50 | 50 |
| 4^{b} | 500 | 500 | 500 | 400 | 75 |

^{a}Second dose started on day 4 in cohorts 1-3;
^{b}Second dose started on day 5 in cohort 4

Endpoints were safety (clinical laboratory test results, adverse events, electrocardiogram (DCG) parameters, physical examination findings) and tolerability, and PK.

Demographics and baseline characteristics of the healthy volunteers for all cohorts are summarized in Table 8.

TABLE 8
Demographics of Participants in Healthy
Subjects MAD Study (n = 10/group)

Compound I Second
(Maintenance) Doses

| Variable | 15 (mg) | 25 (mg) | 50 (mg) | 75 (mg) |
|---|---|---|---|---|
| Age, years | 43.7 | 39.2 | 36.1 | 38.6 |
| (mean, SD) | (9.12) | (8.12) | (9.35) | (10.53) |
| Male, n (%) | 6 | 7 | 7 | 9 |
| | (60.0) | (70.0) | (70) | (90) |

Race, n (%)

| | | | | |
|---|---|---|---|---|
| White | 7 | 4(40) | 8 | 3 |
| | (70.0) | | (80) | (30) |
| Black or African | 3 | 5 | 2 | 7 |
| American | (30.0) | (50) | (20) | (70) |

TABLE 9
Overview of TEAEs in Healthy Subjects
MAD Study Population (n = 10/dose)

| | Compound I Second | | | |
|---|---|---|---|---|
| | (Maintenance) Doses | | | |
| Statistics | 15 mg | 25 mg | 50 mg | 75 mg |
| Number of TEAEs | 8 | 5 | 17 | 3 |
| Number of participants with | 4 | 3 | 7 | 3 |
| at least 1 TEAE, n (%) | (40.0) | (30.0) | (70) | (30.0) |
| Number of TEAEs grade ≥3 | 0 | 0 | 1 | 0 |
| Number of drug-related TEAEs | 1^{b} | 2^{c} | 2^{d} | 0 |
| Number of participants with | 1 | 1 | 1 | 0 |
| at least 1 drug-related TEAE, | (10.0) | (10.0) | (10.0) | |
| n (%) | | | | |
| Number of TEAEs leading to | 0 | 0 | 0 | 0 |
| study drug discontinuation | | | | |
| Number of SAEs | 0 | 0 | 0 | 0 |

^{a} Data presented are still blinded.
^{b}One drug-related TEAE was grade 1 somnolence in 1 participant.
^{c}One participant had 2 drug-related TEAEs vomiting and nausea in 1 participant.
^{d}One participant had 2 TEAEs of headaches and dizziness, MAD, multiple ascending dose; SAE, serious adverse event; TEAE, treatment-emergent adverse event.

Study drug was well tolerated. The safety data was not analyzed by cohort at this time since the study is ongoing and is blinded. A grade 4 increased CPK was reported, but was attributed to extreme exercise at the end of the study and therefore was not related to study drug.

Preliminary whole blood PK parameters (geometric mean and % CV) and hemoglobin occupancy in healthy volunteers of the 15 mg, 25 mg, 50 mg, and 75 mg MAD cohorts are summarized in Table 10. Plasma PK parameters are presented in Table 11.

TABLE 10
Whole Blood PK Parameters in Healthy Subjects
after Repeated Administration of Compound I

| | Day 14 | Day 14 | Day 14 | Day 14 |
|---|---|---|---|---|
| Parameter,^{a} mean | (15 mg) | (25 mg) | (50 mg) | (75 mg) |
| Cₘₐₓ, μg/mL | 130 | 141 | 236 | 308 |
| (CV %) | (14.3) | (16.6) | (16.3) | (14.0) |
| tₘₐₓ,^{b} | 6 | 2 | 3 | 3 |
| h (range) | (0.5-24) | (1-24) | (0-8) | (0-6) |
| AUC₀₋₂₄, μg · h/mL | 2840 | 3090 | 5140 | 6760 |
| (CV %) | (13.7) | (15.8) | (16.4) | (15.4) |
| Cₘᵢₙ μg/mL (CV %) | 121 | 135 | 210 | 297 |
| | (16.2) | (17.4) | (20.7) | (15.2) |
| B:P ratio^{c} | 182 | 194 | 149 | 140 |
| % Hb occupancy | 16.0^{d} | 16.3 | 25.8 | 35.2 |
| (CV %) | (14.3) | (17.4) | (14.9) | (13.9) |

^{a}Calculations based on nominal (anticipated) collection times, not actual.
^{b}tₘₐₓ reported as median.
^{c}B:P = mean AUC_{0-24 blood}:mean AUC_{0-24 plasma}.
^{d}To achieve a similar Hb occupancy with voxelotor, more than 300 mg MAD was needed. AUC0₋₂₄, area under the curve to 24 hours post-dose; Cₘₐₓ, maximal observed concentration; CV, coefficient of variation; Hb, hemoglobin; MAD, multiple ascending dose; PK, pharmacokinetics; SCD, sickle cell disease; t_{1/2}, half-life; tₘₐₓ time of maximal observed concentration.

TABLE 11
Plasma PK Parameters of in Healthy Subjects
After Repeated Administration of Compound I

| | Day 14 | Day 14 | Day 14 | Day 14 |
|---|---|---|---|---|
| Parameter^{a}, mean | (15 mg) | (25 mg) | (50 mg) | (75 mg) |
| Cₘₐₓ, μg/mL | 0.83 | 0.90 | 1.99 | 2.74 |
| (CV %) | (17.4) | (34.9) | (22.9) | (21.5) |
| tₘₐₓ,^{b} | 1 | 1.5 | 1 | 1.0 |
| h (range) | (0, 3) | (0.5, 24) | (0.5, 4) | (1, 2) |
| AUC₀₋₂₄, | 15.6 | 15.9 | 34.5 | 48.6 |
| μg · h/mL(CV %) | (16.1) | (23.2) | (24.1) | (18.0) |
| Cmin μg/mL (CV %) | 0.68 | 0.66 | 1.45 | 2.22 |
| | (18.8) | (26.8) | (25.3) | (14.7) |

^{a}Calculations based on nominal (anticipated) collection times, not actual.
^{b}tₘₐₓ reported as median.
^{c}B:P = mean AUC_{0-24 blood}:mean AUC_{0-24 plasma}.
^{d}To achieve a similar Hb occupancy with voxelotor, more than 300 mg MAD was needed. AUC0₋₂₄, area under the curve to 24 hours post-dose; Cₘₐₓ, maximal observed concentration; CV, coefficient of variation; Hb, hemoglobin; MAD, multiple ascending dose; PK, pharmacokinetics; SCD, sickle cell disease; t_{1/2}, half-life; tₘₐₓ time of maximal observed concentration.

### Example 3: Sickle Cell Disease (SCD Cohort)

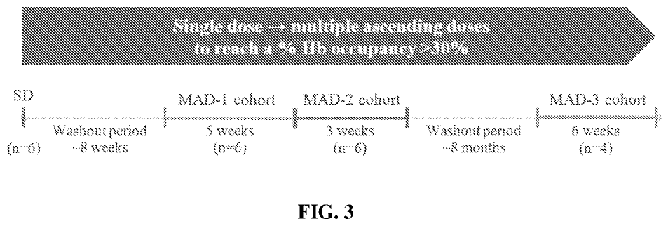

The study design of the single dose (SD) and multiple ascending dose (MAD-1, MAD-2, and MAD-3) cohorts of subjects having Sickle Cell Disease is shown in FIG. 3.

Patients with HbSS (homozygous for SCD), aged 18 to 60 years old, exhibiting baseline Hb ≥5.5 g/dL and ≥10.5 g/dL, and having no vaso-occlusive crises (VOCs) or transfusions within 30 days of screening, were administered a single dose of 100 mg of Compound I (single dose period, SD cohort; see Table 5 of Example 1, SCD 100 mg).

### MAD-1 and MAD-2 Cohorts

After a washout period of at least 56 days (so that elimination PK of Compound I could be assessed), the patients were administered a loading dose of 300 mg per day of Compound I for one day followed by 200 mg per day of Compound I for one day, and then were administered a maintenance dose of 50 mg per day of Compound I for five weeks (“MAD-1 Cohort”). The same patients then received another loading dose of 500 mg per day of Compound I for one day followed by 400 mg per day of Compound I for one day, and then were administered another maintenance dose of 100 mg per day of Compound I for three weeks (“MAD-2 Cohort”). The total treatment period of the MAD-1 Cohort and MAD-2 Cohort was 8 weeks.

Primary endpoints were safety and tolerability. Key secondary endpoints included PK and the relationship between time-matched Compound I concentrations and changes in clinical measures of anemia and hemolysis from baseline.

Pharmacodynamic (PD) markers such as p20 (partial pressure of O₂ at which Hb is 20% saturated with O₂) and p50 (partial pressure of O₂ at which Hb is 50% saturated with O₂) were determined by measuring oxygen equilibrium curves (OECs) after 2 dose escalations. OECs were generated by hemoximetry, which relates the extent of Hb-O₂ saturation to the partial pressure of O₂ (pO₂) and measures the binding affinity of O₂ to Hb.

Demographics and baseline characteristics of the SCD cohort are summarized in Table 12. The treatment-emergent adverse events of the SCD cohort are summarized in Table 13A and Table 13B. The majority of treatment emergent adverse events were grade 1 or 2 and unrelated to study drug. Compound I was safe and well tolerated in both the single ascending dose portion and the multiple ascending dose phases.

TABLE 12
Demographics & Baseline Characteristics
of SCD Study Population (N = 6)

| | |
|---|---|
| Mean age, years (SD) | 20.2 |
| | (2.23) |
| Male, n (%) | 4 |
| | (66.7) |

Race, n (%)

| | |
|---|---|
| Black or African | 5 |
| American | (83.3) |
| Other | 1 |
| | (16.7) |
| HbSS genotype, n (%) | 6 |
| | (100) |
| Hb level, g/dL, mean (SD), | 8.2 (0.4), |
| range | 7.7-9.1 |
| Current hydroxyurea | 6 |
| use, n (%) | (100) |

Number of VOCs within 12
months of screening, n (%)

| | |
|---|---|
| 0 | 2 |
| | (33.3) |
| 1 | 1 |
| | (16.7) |
| 2 | 3 |
| | (50.0) |

Number of transfusions within 12
months of screening, n (%)

| | |
|---|---|
| 0 | 4 |
| | (66.7) |
| 1 | 2 |
| | (33.3) |

TABLE 13A
Overview of TEAEs in SCD (N = 6) for
the Single Dose and Multiple Dose Portions

| | |
|---|---|
| Number of TEAEs | 24 |
| Number of participants with at least | 5 |
| 1 TEAE, n (%) | (83.3) |
| Number of TEAEs grade ≥3 | 5 |
| Number of drug-related TEAEs | 3^{a} |
| Number of participants with at least 1 | 2 |
| drug-related TEAE, n (%) | (33.3) |
| Number of TEAEs leading to study drug | 0 |
| discontinuation | |
| Number of SAEs | 2^{b} |

^{a}The drug-related TEAEs were 1 grade 2 headache and 2 cases of grade 1 diarrhea.
^{b}The 2 SAEs were grade 3 VOCs. HbSS, homozygous for SCD; SAE, serious adverse event; SCD, sickle cell disease; TEAE, treatment-emergent adverse event; VOC, vaso-occlusive crisis.

TABLE 13B
Overview of TEAEs, Single Dose, MAD-1 (50
mg qd), and MAD-2 (100 mg qd) (n = 6)

| | |
|---|---|
| Number of TEAEs | 33 |
| Number of participants with at least | 6 |
| 1 TEAE, n (%) | (100) |
| Number of TEAEs grade ≥3 | 7 |
| Number of drug-related TEAEs | 4 |
| Number of participants with at least | 2 |
| 1 drug-related TEAE, n (%) | (33.3) |
| Number of TEAEs leading to study drug | 0 |
| discontinuation | |
| Number of SAEs | 3 |

The PK profile of a single 100 mg dose in whole blood and plasma was evaluated using standard noncompartmental analysis. The findings from the SCD subjects were compared to those in healthy subjects, as shown in Table 14. The preliminary PK parameter estimates of Compound I in whole blood from SCD subjects differed from healthy subjects in terms of elimination t½, AUC, Tₘₐₓ, and blood to plasma ratio. In brief, the elimination t½ and Tₘₐₓ were nearly 3× faster or occurred earlier, respectively, in SCD than in healthy subjects, and the blood to plasma ratio was about 30% of the values in healthy subjects (most likely due to the fewer RBCs compared to healthy subjects). For the plasma compartment, the differences between the 2 populations were most notable in terms of elimination t1/2, which again was about 2.5 times faster in SCD.

TABLE 14
Comparison of Whole Blood PK Parameters (Geometric
mean CV %) after a 100 mg dose of Compound I
in Healthy Subjects and Subjects with SCD

| | Compound I | Compound I | Compound I | Compound I |
|---|---|---|---|---|
| | in healthy | in SCD | in healthy | in SCD |
| | subjects | subjects | volunteers | subjects |
| Parameter | (n = 6) | (n = 6) | (n = 6) | (n = 6) |
| Matrix | Blood | | Plasma | |
| Cₘₐₓ | 17.3 | 16.2 | 0.714 | 1.01 |
| (μg/mL) | (12.3) | (25.9) | (22.6) | (20.8) |
| Tₘₐₓ | 24 | 8 | 1.0 | 1.5 |
| (h) (median) | (8, 48) | (6, 36) | (0.5, 3) | (1, 2) |
| AUC₀₋ₜ | 14,300 | 4,540 | 68.7 | 67.2 |
| (μg/mL*h) | (14.6) | (29.5) | (17.4) | (14.0) |
| t½ | 29.1 | 9.8 | 25.6 | 10.5 |
| (days) | (22.9) | (13.6) | (22.5) | (22.8) |
| B:P ratio | 216 | 69.3 | NA | NA |
| AUC_{0-inf} | | | | |

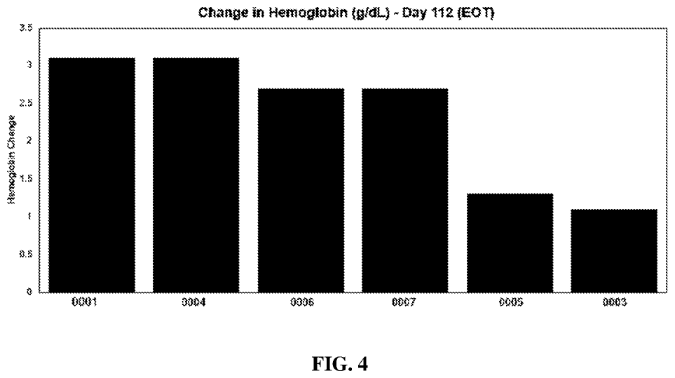

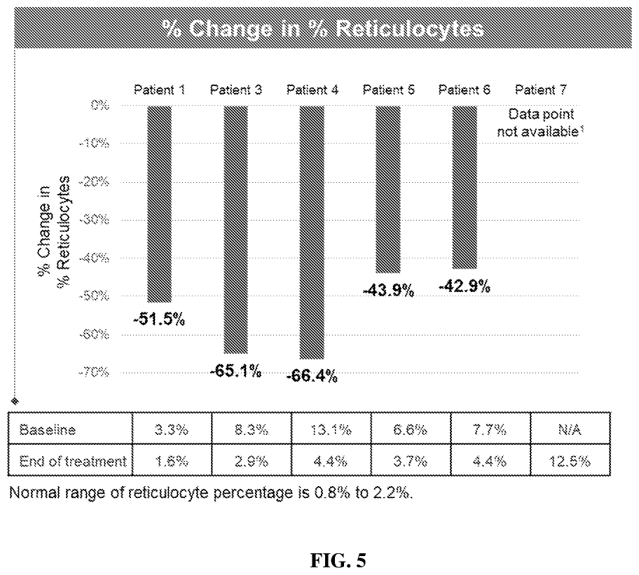

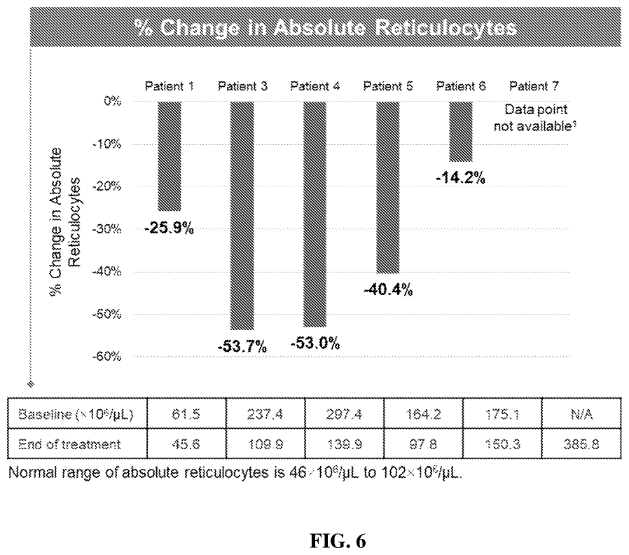

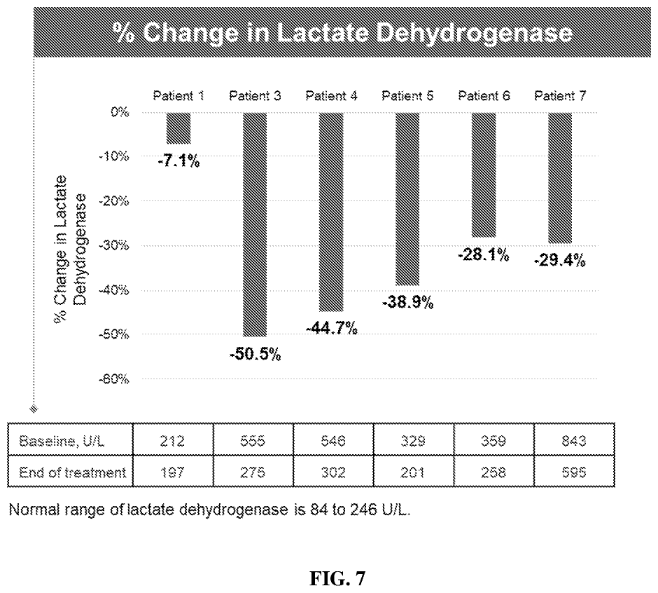

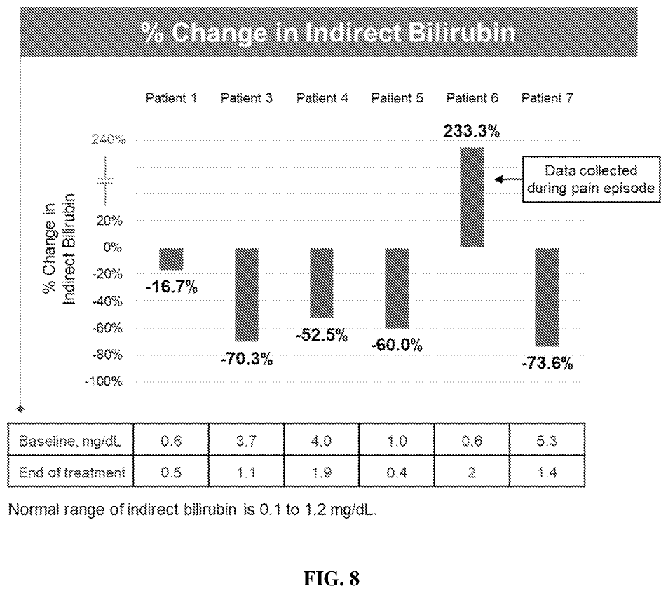

FIG. 4 shows the change in hemoglobin at 8 weeks following two rounds of treatment with of Compound I (day 56 to day 112) in six SCD patients. At the end of the study, Hb increased by up to 1.3 g/dL (mean increase of 2.3 g/dL), and all six subjects experienced improvements in hemolysis markers including reticulocytes (FIG. 5), absolute reticulocytes (FIG. 6), lactate dehydrogenase (LDH) (FIG. 7), and indirect bilirubin (FIG. 8). The mean (SD) percent Hb occupancy, or when trough (Cₘᵢₙ) and apparent peak levels (Cₘₐₓ) of study drug are reached in plasma and whole blood after 5 weeks of 50 mg QD and 3 weeks of 100 mg QD are shown in Table 15. Individual Hb occupancy values for the 100 mg cohort at Cₘₐₓ ranged from 19.7% to 41.8%. When steady state was reached at the end of each treatment period, there was little difference between Cₘᵢₙ and Cₘₐₓ values which is consistent with the findings in healthy subjects. At 100 mg dose level, two out of six patients achieved Hb occupancy >40%. Mean values are consistent with those observed during the weeks preceding the value obtained at the end of once daily 50 mg and 100 mg treatment periods, or at weeks 5 and 7 of repeated dose treatment (maintenance dose).

TABLE 15
Hemoglobin Occupancy (%) after Treatment
with Two Dose Levels of Compound I

| | |
|---|---|
| 50 mg QD × 5 weeks | 100 mg QD × 3 weeks |

| Statistic | Cₘᵢₙ | Cₘₐₓ | Cₘᵢₙ | Cₘₐₓ |
|---|---|---|---|---|
| Mean | 15.30 | 17.66 | 29.80 | 32.59 |
| SD | 5.72 | 7.75 | 8.30 | 9.35 |
| CV % | 37.5 | 44.0 | 27.9 | 28.7 |

The mean increase in hematocrit from baseline to end of week 8 of treatment was 6.5%. Following the last dose of Compound I, the concentrations of study drug were assessed over about 15 weeks and the elimination t1/2 averaged about 10 days. This was accompanied by decreases in mean hemoglobin of 2.2 g/dL. Following this 15-week washout period, the average hematocrit in 5 subjects returned to baseline levels (23.46% vs. 23.02%).

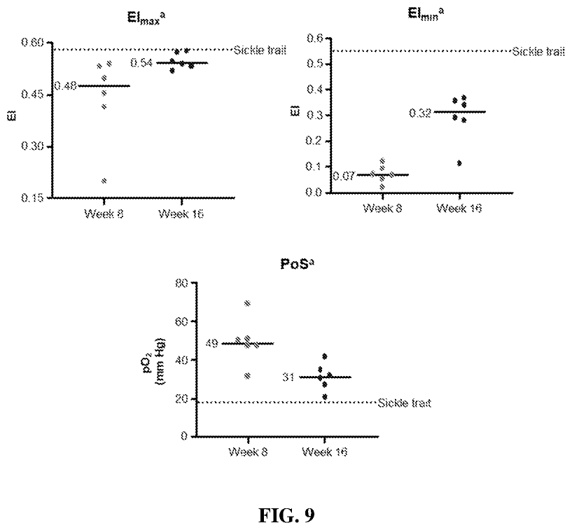

An ektacytometer laser optical rotational red cell analyzer (Lorrca; RR Mechatronics, NL) was used to analyze deformability of red blood cells using a defined value of shear stress with an increasing osmotic gradient (Osmoscan) as well as a newer technology to subject these cells to gradual deoxygenation (Oxygenscan). Improvements in red blood cell health were seen in all patients as demonstrated by Oxygenscan testing (FIG. 9).

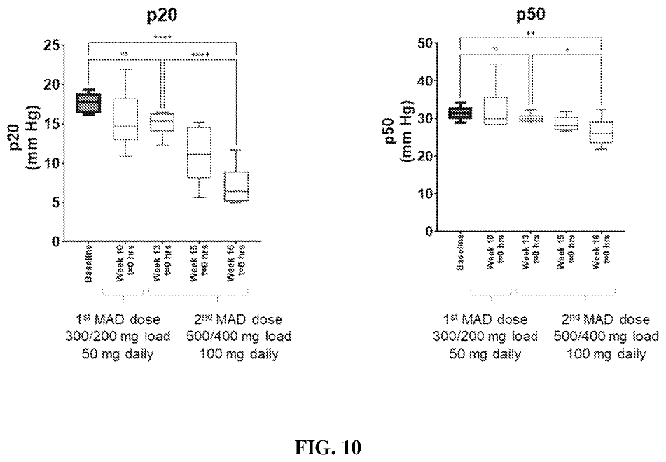

Hemoximetry box blots (FIG. 10) show p20 and p50 values of blood collected from patients with SCD (n=6) during dosing with Compound I. The p20 and p50 values for baseline (week 8) to week 10, and weeks 13 to 16 represent the 50 mg and 100 mg Compound I dosing periods, respectively. Both the p20 and p50 values decreased from baseline, indicating Hb modification of blood by Compound I and a dose-dependent increase in Hb-O₂ affinity. These changes in p20 and p50 values during treatment with Compound I correspond to left shifts of the OECs relative to baseline (no treatment). The change in p20 per dose is larger than that with p50 and represents a more sensitive measure of Hb modification and indicator of increased Hb-O₂ affinity. These results indicate that measurements of OECs allow for sensitive monitoring of the degree of increase in Hb-O₂ affinity and represent Hb modification by Compound I during treatment.

### MAD-3 Cohort

Four out of the six patients who participated in the MAD-1 and MAD-2 studies, as described above, proceeded to the MAD-3 study (n=4). While data for patient 003 is provided, the patient did not adhere to the MAD-3 dosing regimen.

Following the conclusion of the MAD-2 study and a washout period of about 8 months, the selected patients (n=4) were administered a loading dose of 300 mg BID for 4 days, followed thereafter by a maintenance dose of 150 mg QD (MAD-3 Cohort).

Primary endpoints were safety and tolerability. Key secondary endpoints included PK and the relationship between time-matched Compound I concentrations and the changes in clinical measures of anemia and hemolysis from baseline.

Pharmacodynamic (PD) markers such as p20 and p50 were determined by measuring OECs, which were generated by hemoximetry.

Demographics and baseline characteristics of the MAD-3 SCD cohort are summarized in Table 16. The treatment-emergent adverse events of the MAD-3 SCD cohort are summarized in Table 17.

TABLE 16
Demographics & Baseline Characteristics of SCD Patients
who Participated in MAD-3 Cohort (N = 4)

| | |
|---|---|
| Mean age, years (SD) | 19.8 |
| | (18.0, 23.0) |
| Male, n (%) | 2 |
| | (50.0) |

Race, n (%)

| | |
|---|---|
| Black or African | 3 |
| American | (75.0) |
| Other | 1 |
| | (25.0) |
| HbSS genotype, n (%) | 4(100) |
| Hb level, g/dL, mean (SD), | 9.2 (0.4), |
| range | 8.6-9.6 |
| Current hydroxyurea use, | 4 |
| n (%) | (100) |

Number of VOCs within 12
months of screening

| | |
|---|---|
| 0 | 2 |
| | (50.0) |
| 1 | 1 |
| | (25.0) |
| 2 | 3 |
| | (25.0) |

Number of transfusions within
12 months of screening

| | |
|---|---|
| 0 | 3 |
| | (75.0) |
| 1 | 1 |
| | (25.0) |

TABLE 17
Overview of TEAEs in the MAD-3 SCD Patients (N = 4)^{a}

| | |
|---|---|
| Number of TEAEs | 6 |
| Number of participants with at | 3 |
| least 1 TEAE, n (%) | (75) |
| Number of TEAEs grade ≥3 | 0 |
| Number of drug-related TEAEs | 1 |
| Number of participants with at | 1 |
| least 1 drug-related TEAE, n (%) | (25) |
| Number of TEAEs leading to study | 0 |
| drug discontinuation | |
| Number of SAEs | 0 |

^{a}Two patients did not move forward with the MAD-3 portion of the study because they initiated disease-modifying therapy before the start of MAD-3.

The hematocrit and Hb levels at week 6 in patients who participated in the MAD-3 cohort are shown in Table 18.

TABLE 18
Hematocrit and Hb Levels in MAD-3
Cohort SCD Patients at Week 6

| | Hematocrit | Hb levels |
|---|---|---|
| Patient | (%) | (g/dL) |
| 0001 | — | 12.9 |
| 0003 | 27.6 | 8.9 |
| 0004 | 37.8 | 13.6 |
| 0005 | 30.9 | 10.9 |

The concentrations of Compound I in whole blood and plasma at week 6 in patients who participated in the MAD-3 cohort are shown in Table 19.

TABLE 19
Whole Blood and Plasma PK (Cₘₐₓ) in
MAD-3 Cohort SCD Patients at Week 6

| | Whole Blood | Plasma |
|---|---|---|
| | (Cₘₐₓ) | (Cₘₐₓ) |
| | Concentration | Concentration |
| Patient | (μg/mL) | (μg/mL) |
| 0001 | 322 | 6.98 |
| 0003 | 36.7 | 1.61 |
| 0004 | 259 | 3.83 |
| 0005 | 308 | 7.61 |

The % Hb occupancy by Compound I at week 6 in patients who participated in the MAD-3 cohort are shown in Table 20.

TABLE 20
% Hb Occupancy in MAD-3 Cohort SCD Patients at Week 6

| Patient | Hb Occupancy (%) |
|---|---|
| 0001 | 48 |
| 0003 | 7 |
| 0004^{a} | 31 |
| 0005 | 46 |

^{a}Patient had increases from baseline in Hb (3 g/dL) and hematocrit (8.1%), which lower the occupancy value.

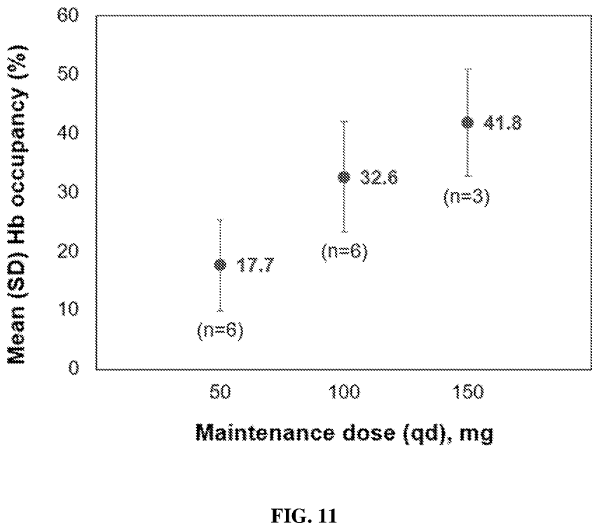

The mean % Hb occupancy of patients who participated in all three cohorts, MAD-1, MAD-2, and MAD-3 respectively, are shown in FIG. 11.

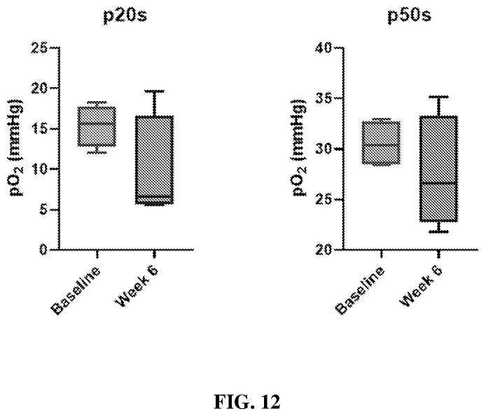

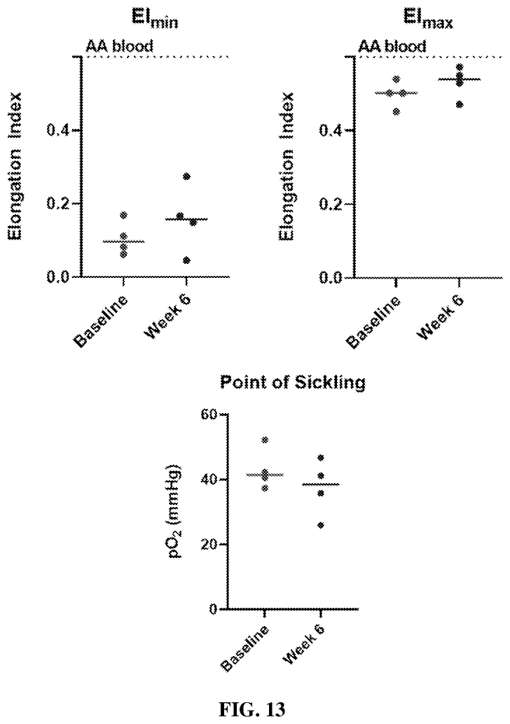

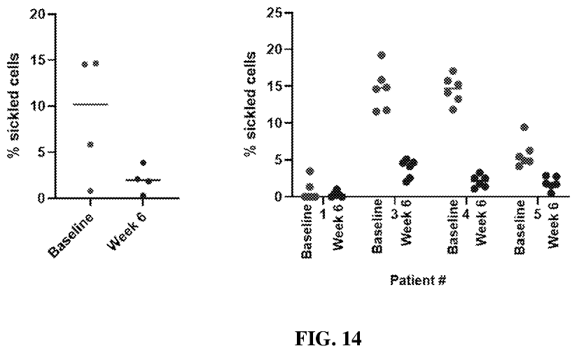

Hemoximetry box blots showing p20 and p50 values of blood collected from patients who participated in the MAD-3 cohort are shown in FIG. 12. Improvement in red blood cell health were also observed in the same MAD-3 cohort of SCD patients as shown in FIG. 13. A summary of changes in percentage of sickled red blood cells for all MAD-3 cohort SCD patients are shown in FIG. 14.

### CONCLUSION

In adults with SCD, treatment with Compound I using the dosing regimens as described herein wherein maintenance doses were 100 mg and 150 mg QD led to a mean Hb occupancy >30%, increased hematocrit, and increased Hb levels. Daily maintenance doses of Compound I at 150 mg were well tolerated in adults with SCD. Results from hemoximetry, ektacytometry, and peripheral blood smears suggest that Compound I maintenance doses of 100 mg and 150 mg improve red blood cell health.

### Example 4: Model-Informed Dose Selection of Compound I for Repeat Dosing

A “learn and confirm” model-informed approach was used to select the first dose for studies in healthy volunteers and patients with sickle cell disease (SCD) and comprised 2 steps: (1) a non-mechanistic population pharmacokinetics (popPK) model was developed using available data and continually refined to characterize plasma and whole-blood concentrations, and (2) Monte Carlo simulations were used to choose an appropriate first (loading) dose to achieve the targeted Hb occupancy of the second (maintenance) dose within the first week of treatment in subsequent cohorts.

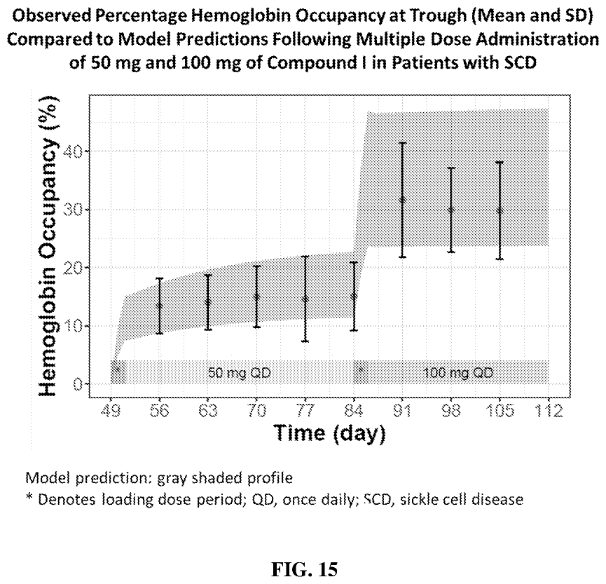

The model-selected first doses achieved steady-state concentrations within 1 week and were maintained by second doses of 50 mg as demonstrated by the consistent observed trough concentrations compared with the model predictions over the treatment duration as shown in FIG. 15. The sequence was repeated with new model-selected first doses achieving steady-state concentrations within 1 week and were maintained by second doses of 100 mg.

Using the popPK model, a first (loading) dose of 300 mg twice daily (BID) for 4 days followed by second (maintenance) dose of 150 mg per day thereafter was selected to achieve a targeted Hb occupancy of 38% to 59%.

### Example 5: A Phase 2/3 Randomized and Multicenter Study of Compound I Administered Orally to Participants with Sickle Cell Disease and an Open-Label Pharmacokinetics Study in Pediatric Participants with Sickle Cell Disease

This example describes a three-part Phase 2/3 study of orally administered Compound I to participants with SCD.

### Part A

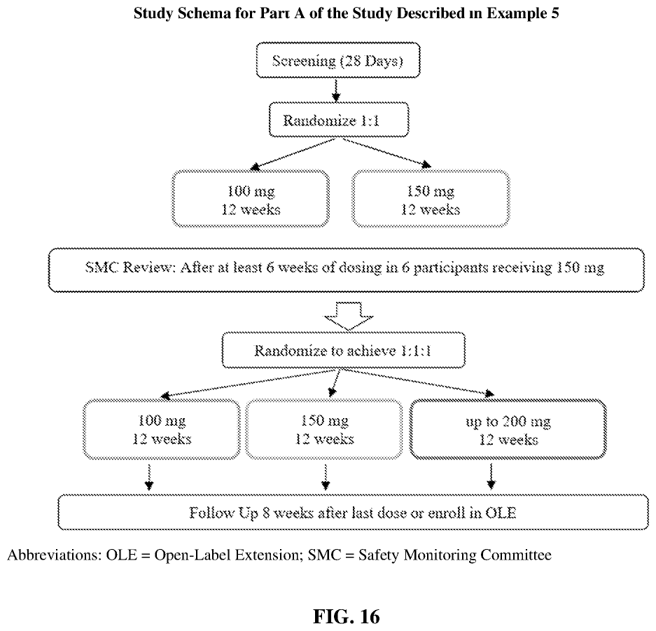

Part A of this study is a Phase 2 randomized, open-label, dose-finding study in approximately 60 adult participants with SCD. The primary objective of Part A of this study is to assess the effects of Compound I in adult participants with SCD as measured by change in hemoglobin. A study schema for Part A of the study is shown in FIG. 16.

Participants are screened for eligibility in the study from Day −28 through Day −1. Eligible participants are randomized on Day 1 based on a 1:1 (100 mg: 150 mg) allocation and will receive their initial dose (loading dose) of Compound I. After completion of Day 1 assessments and subsequent loading doses, participants will continue with administration of their maintenance dose daily through Week 12.

After completion of at least 6 weeks of dosing of 6 adult participants from the 150 mg treatment group, safety, tolerability, and available PK, Hb occupancy, and PD data is reviewed to assess whether the maintenance dose may be increased up to a maximum of 200 mg.

After a determination has been made to include a higher dose treatment arm, participants enrolled thereafter are randomized 1:1:1 (100 mg: 150 mg: up to 200 mg of Compound I respectively) or adaptive method to ensure balance and receive a loading dose regimen over 4 days followed by daily maintenance doses through Week 12. Loading doses to be used in this study will not exceed 2200 mg within 24 hours period.

The proposed loading and maintenance doses to achieve the desired exposures and targeted % Hb occupancies for each dose group are shown in Table 21 below.

TABLE 21
Model-Predicted Dosing Regimen

| Dose | | | Mean Targeted | Mean Targeted |
|---|---|---|---|---|
| Group | Loading | Maintenance | % Hb Occupancy | % HbOccupancy |
| (N = 20) | Doses | Dose | (Model 1)^{a} | (Model 2)^{b} |
| 1 | 200 mg | 100 mg QD | ~36% | ~32% |
| | BID × | | | |
| | 4 days | | | |
| 2 | 300 mg | 150 mg QD | ~54% | ~46% |
| | BID × | | | |
| | 4 days | | | |
| 3 | 400 mg | 200 mg QD | ~71% | ~57%^{c} |
| | BID × | | | |
| | 4 days | | | |

^{a}Model 1 is described in Example 4 above.
^{b}Mean targeted % Hb occupancy values from Model 2 incorporate changes in hemoglobin (ΔHb) and hematocrit (ΔHct) based on observed changes in Hb and Hct in SCD patients treated with Compound I (MAD-2 and MAD-3 cohorts as described above).
^{c}Value calculated based on assumption of an equivalent change in Hb and Hct as observed in the MAD-3 cohort as described above.

The primary endpoint of Part A of the study is to assess the change from baseline in hemoglobin through Week 12.

The secondary endpoint of Part A of the study is to evaluate the effects of Compound I on total hemoglobin and clinical measures of hemolysis, and to evaluate the safety and tolerability as well as the PK and PD properties of multiple dose Compound I administration. Additional secondary endpoints of Part B of the study also include:
Proportion of participants with an increase from baseline of >1 g/dL in Hb through Week 12
Percent change from baseline in hemolysis measures, including indirect bilirubin, absolute and % reticulocytes, and LDH through Week 12
Incidence of adverse events, changes in laboratory assessments, ECGs, and vital signs
Effect on Hb OEC as measured by p50 through Week 12, and
AUC₀₋₂₄, Cₘₐₓ, Tₘₐₓ, blood to plasma (B:P) ratios of these PK parameters (except Tₘₐₓ) after the first dose.

The exploratory endpoint of Part A of the study is to evaluate the effects of Compound I on neurocognitive function, erythropoietin (EPO) levels, quality of life (QOL) assessments, RBC deformability, RBC mitochondrial content, VOCs, and other biomarkers. Additional exploratory endpoints of Part A of the study include:
Change from baseline through Week 12 in the Executive abilities composite score (using Dimensional Change Card Sort Test, Flanker Inhibitory Control and Attention Test, and Pattern Comparison Processing Speed Test as assessed by the NIH Toolbox Cognition Module).
Change from baseline in EPO levels through Week 12.
Change from baseline in QOL assessments through Week 12 including PGI-C, CGI-C, EQ-5D-5L, and PROMIS-29.
Change from baseline in RBC deformability and RBC mitochondrial content through Week 12.
Annualized rate of VOC through Week 12.
Time to first VOC from randomization.
Change from baseline in other biomarkers at Week 12 (including genomic analysis, membrane fragility, flow adhesion, and protein markers by multiplex assay which may include ICAMs, VCAMs, P-selectin, E-selectin, ACR, KIM, and markers of hemolysis such as levels of haptoglobin, hemopexin, soluble C3, and cell free hemoglobin).

### Part B

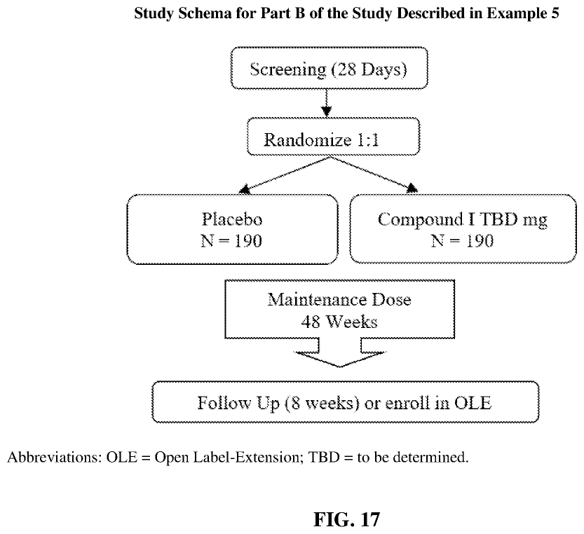

Part B is a Phase 3, randomized, double-blind, placebo-controlled study in adult and pediatric participants. Part B will initiate after selection of the optimum dose from Part A of the study. Part B of the study will assess the safety and efficacy of Compound I in increasing the Hb response in approximately 380 adult and pediatric participants with SCD, with approximately 190 participants receiving placebo and approximately 190 participants receiving Compound I optimal dose selected from Part A of the study. A study schema for Part B of the study is shown in FIG. 17.

The primary objective of Part B of the study is to assess the effects of an optimum Compound I dose as compared to placebo in adult and pediatric participants with SCD as measured by hemoglobin response.

In Part B of the study, participants are screened for eligibility in the study from Day −28 through Day −1. Eligible adult participants are randomized on Day 1 based on a 1:1 (Compound I: Placebo) allocation and will receive their initial dose (loading dose) at the clinical site. After completion of Day 1 assessments and subsequent loading doses, participants will continue with administration of their maintenance dose daily from Week 1 through Week 48. Participants will return to the clinical site for assessments of safety, efficacy, and PK/PD. The incidence of VOC events will be collected every 4 weeks. After completion of treatment at Week 48 (Day 337), participants will return for final follow-up visit 8 weeks later (Week 56, Day 393), approximately 5 half-lives after the last dose. Participants may choose to enroll in an OLE study under a separate protocol after completing Week 48 visits. Participants not entering the OLE study will be followed for approximately 8 weeks after completing dosing to further assess safety.

The primary endpoint of Part B of the study is to assess the proportion of participants with an increase from baseline of >1 g/dL in Hb at Week 48.

The secondary endpoint of Part B of the study is to evaluate the effects of Compound I compared to placebo on total hemoglobin, clinical measures of hemolysis, and relevant clinical outcomes, and to evaluate the safety and tolerability of 48 weeks of daily administration of Compound I. Additional secondary endpoints also include:
Annualized rate of VOC through end of Week 48.
Proportion of participants with an increase from baseline of >1 g/dL in Hb at Week 24.
Change from baseline in Hb through Weeks 24 and 48.
Percent change from baseline in hemolysis measures, including indirect bilirubin, absolute and % reticulocytes, and LDH through Week 48.
Change from baseline in QOL assessments through Week 48 including PGI-C, CGI-C, and PROMIS-29 physical function, anxiety, depression, fatigue, social function, pain interference, and pain intensity.
Incidence of adverse events, changes in laboratory assessments, ECGs, and vital signs.
Trough plasma and blood concentrations, B:P ratio.
Percent Hb occupancy through Week 48.
Time to first VOC from randomization.
Time to first Hb increase from baseline of >1 g/dL during the study.

The exploratory endpoint of Part B of the study is to evaluate the effects of Compound I as compared to placebo on neurocognitive function, EPO levels, QOL assessments, RBC deformability, RBC mitochondrial content, VOCs, and renal biomarkers. Additional exploratory endpoints also include:
Change from baseline in other biomarkers through Week 48 (including genomic analysis [optional] membrane fragility, flow adhesion, and protein markers by multiplex assay which may include ICAMs, VCAMs, P-selectin, E-selectin, ACR, KIM, and markers of hemolysis such as levels of haptoglobin, hemopexin, soluble C3, and cell free hemoglobin).
Change from baseline through Week 48 in the Executive abilities composite score (using Dimensional Change Card Sort Test, Flanker Inhibitory Control and Attention Test, and Pattern Comparison Processing Speed Test) as assessed by the NIH Toolbox Cognition Module.
Change from baseline in EPO levels through Week 48.
Change from baseline in QOL assessments through Week 48 including EQ-5D-5L.
Change from baseline in RBC deformability and mitochondrial content through Week 48.

### Part C

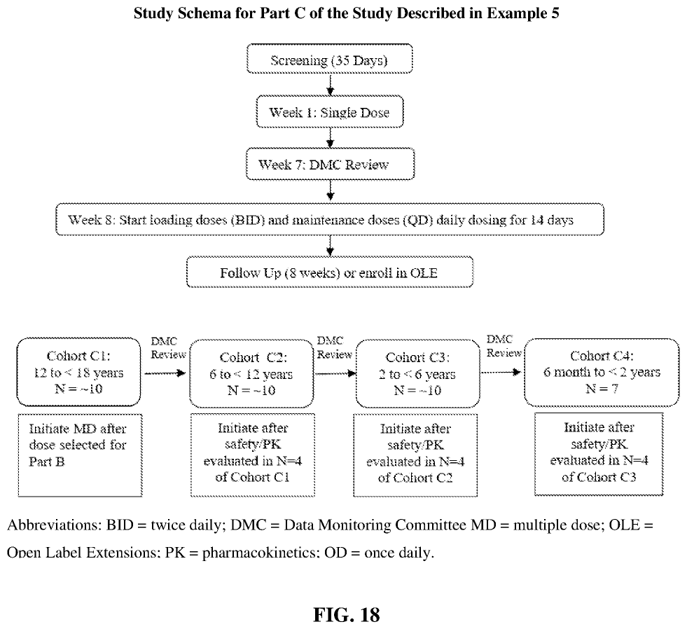

Part C of the study is a single arm, open-label, multi-dose PK study in four age-group cohorts of pediatric participants with SCD, and the multi-dose portion of Part C of the study will begin after the dose has been selected for Part B of the study. A study schema for Part B of the study is shown in FIG. 18.

Pediatric participants are assessed for study eligibility during a 35-day Screening Period (additional time allowed in Screening due to blood draw volume restrictions in pediatric participants). On study Day 1, eligible pediatric participants will receive a single dose of Compound I (100 mg dose in cohort C1, dose level for cohorts C2, C3, and C4 to be determined based on emerging data). Safety, tolerability, and available PK data of Compound I will be assessed approximately 7 weeks after at least 4 participants have completed single-dose (SD) administration in each Cohort and provide a recommendation on proceeding with multiple-dose (MD) dosing. Pediatric participants will receive a loading dose regimen over 4 days (starting on MD Day 1) followed by once daily dosing for a total treatment duration of 14 days. Doses given in the MD portion will be informed by emerging clinical trial data and will not exceed dose levels established as safe in adult clinical trials or previous pediatric cohorts. Participants may choose to enroll in an open-label extension (OLE) study under a separate protocol after completing MD Week 2 visits. Participants not entering the OLE study will be followed for approximately 8 weeks after completing dosing to further assess safety.

Part C is composed of four cohorts:
Cohort C1 includes approximately 10 participants aged 12 to less than 18 years, at least 30% of whom must be 12 to less than 15 years old.
Cohort C2 includes approximately 10 participants aged 6 to less than 12 years, at least 30% of whom must be 6 to less than 9 years old.
Cohort C3 includes approximately 10 participants aged 2 to less than 6 years.
Cohort C4 includes approximately 7 participants aged 6 months to less than 2 years.

Cohorts C2-C4 will be initiated after completion of 14 days of MD dosing and Data Monitoring Committee (DMC) review of safety, tolerability, and PK data has been evaluated in at least 4 participants of the preceding Cohort. Up to 10 additional participants may be enrolled in each cohort if needed to evaluate additional dose levels and/or to further characterize PK or safety if deemed necessary. Participants from Part C will not be eligible to enroll in Part B of the study.

### Inclusion Criteria

Key inclusion criteria for Parts A, B, and C include:
Male or female with SCD. Documentation of SCD genotype homozygous for sickle cell allele (HbSS) or double heterozygote for sickle hemoglobin (HbS) and β-0 thalassemia (HbSB) may be based on history of laboratory testing or must be confirmed by laboratory testing during Screening.
Hb≥5.5 and ≤10.5 g/dL during Screening and considered stable by the Investigator.
For participants taking hydroxyurea (HU) and/or L-glutamine, the dose must be stable for at least 90 days prior to signing the ICF or assent and with no anticipated need for dose adjustments during the study in the opinion of the Investigator.
Female participants of child-bearing potential, must agree to use highly effective methods of contraception or practice abstinence from study start to 120 days after the last dose of study drug. Males who are not surgically sterile with partners of childbearing potential must agree to use a highly effective method of birth control during the study and for 120 days after the last dose of study drug. In addition, males who are not surgically sterile with a partner who is pregnant must agree to condom use or maintain sexual abstinence during the study and for 120 days after the last dose of study drug.
Female participants of child-bearing potential must have a negative pregnancy test before administration of study drug.
Participant has provided documented informed consent (Part A and Part B for adult participants) or written informed parental/guardian consent and participant assent has been obtained per institutional review board (IRB)/Ethics Committee (EC) policy and requirements, consistent with International Council for Harmonisation (ICH) guidelines (Part B for pediatric participants and Part C).

Additional key inclusion criteria for Part A include:
Age 18 to ≤65 years, inclusive at Screening
Males must agree to not donate sperm from study start through 120 days after final dose.

Additional key inclusion criteria for Part B include:
Age 12 to ≤65 years, inclusive at Screening. Participants 12 to <18 years will only be enrolled in Part B after evaluation of safety in Part C Cohort C1.
Males must agree to not donate sperm from study start through 120 days after the final dose
More than or equal to 2 and ≤10 VOCs within 12 months of Screening.

### Exclusion Criteria

The key exclusion criteria for Parts A, B, and C include:
More than 10 VOCs within 12 months of Screening.
Female participant who is breastfeeding or pregnant.
Receiving regularly scheduled RBC transfusion therapy (also termed chronic, prophylactic, or preventive transfusion) or has received an RBC or exchange transfusion for any reason within 90 days of Day 1.
Hospitalized for sickle cell crisis or other vaso-occlusive event within 14 days of signing the ICF.
Screening laboratory test of alanine aminotransferase (ALT)>4×upper limit of normal (ULN) for age.
Acute illness or clinically significant bacterial, fungal, parasitic, or viral infection which requires therapy, including acute bacterial infection requiring antibiotics within 14 days prior to the study drug administration.
Participants known to have active hepatitis A, B, or C or human immunodeficiency virus (HIV).
Participants with active, symptomatic coronavirus disease 2019 (COVID-19) infection.
Estimated glomerular filtration rate (eGFR)<60 mL/min/1.73 m² at the Screening Visit, calculated by the central laboratory, or is on chronic dialysis.
History of malignancy within the past 2 years prior to treatment Day 1 requiring chemotherapy and/or radiation (with the exception of local therapy for non-melanoma skin malignancy).
History of unstable or deteriorating cardiac or pulmonary disease within 6 months prior to consent.
Has received COVID-19 vaccine (first dose, second dose, or booster dose), authorized by regional regulatory authority, within 7 days prior to Day 1
Received EPO or other hematopoietic growth factor treatment within 28 days of signing ICF or is anticipated to require such agents during the study.
Current or recent use of voxelotor. Recent use is defined as within 10 days prior to Day 1.
Current or recent use of crizanlizumab. Recent use is defined as within 90 days prior to Day 1.
Ongoing or recent use of strong or moderate inducers of cytochrome P450 (CYP) or CYP3A4/CYP3A5. Recent is defined as within 5 elimination half-lives or 14 days, whichever is longer prior to Day 1.
Ongoing or recent use of strong or moderate inhibitors of CYP3A4/CYP3A5. Recent is defined as within 5 elimination half-lives prior to Day 1.
Ongoing or recent use of the P-glycoprotein substrates digoxin or dabigatran. Recent is defined as within 5 elimination half-lives prior to Day 1.
Use of prohibited prescription or nonprescription drugs and dietary supplements (including herbal and alternative medications). Marijuana use is allowed, except for 24 hours prior to neurocognitive assessments as outlined in the Schedule of Assessments.
Known allergy to Compound I or other Hb polymerization inhibitors.
History of severe allergic reaction (including anaphylaxis) to any substance, or previous status asthmaticus.
Unlikely to comply with the study procedures

Additional key exclusion criteria for Part C only include:
History of stroke or meeting criteria for primary stroke prophylaxis (history of two transcranial doppler [TCD] measurements≥200 cm/sec by nonimaging TCD or ≥185 cm/sec by TCDi).

### Dose

Dose selection is based on safety and PK results from previous studies as described in Examples 1, 2, and 3.

A loading dose is administered followed by daily maintenance dosing for multi-dose regimens.

### Duration of Treatment

In Part A, all adult participants will receive maintenance dosing through approximately Week 12.

In Part B, all adult and pediatric participants will receive maintenance dosing for 48 weeks.

In Part C, pediatric participants in Cohort C1 will receive a single dose of 100 mg followed by daily dosing starting at approximately Week 8 continuing for 14 days. The multi-day dosing regimen for all Part C cohorts and single dose levels for Cohorts C₂ to C₄ will be informed by emerging data. Doses utilized will not exceed doses determined to have acceptable tolerability in adults or older age pediatric cohorts. Part C cohorts C3 and C4 will be administered Compound I as either a powder for oral solution, or a dispersible tablet.

All patents and other references cited in the specification are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

What is claimed is:

1. A method for treating sickle cell disease in a subject in need thereof, comprising administering to the subject a first dose of Compound I of formula: 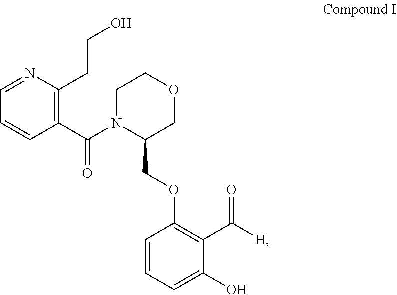
or a pharmaceutically acceptable salt thereof, for one, two, three, four, five, six, or seven days,
followed thereafter by a second dose of Compound I, or a pharmaceutically acceptable salt thereof, wherein the administrations of the first dose and the second dose provide about 25% to about 60% hemoglobin occupancy by Compound I.

2. The method of claim 1, wherein the administrations of the first dose and the second dose provide about 30% to about 50% hemoglobin occupancy by Compound I.

3. A method for treating sickle cell disease in a subject in need thereof, comprising administering to the subject a first dose of about 200 mg per day to about 1600 mg per day of Compound I of formula: 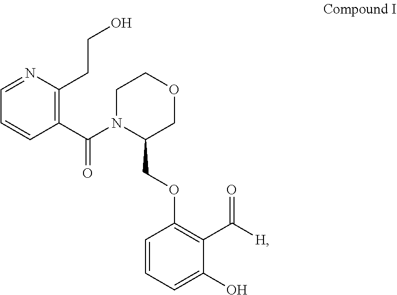
or a pharmaceutically acceptable salt thereof, for one, two, three, four, five, six, or seven days,
followed thereafter by a second dose of about 25 mg per day to about 500 mg per day of Compound I, or a pharmaceutically acceptable salt thereof.

4. The method of claim 3, comprising administering to the subject a first dose of about 300 mg per day to about 1500 mg per day of Compound I of formula: 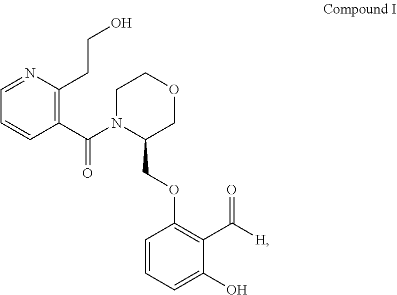
or a pharmaceutically acceptable salt thereof, for one, two, three, four, five, six, or seven days,
followed thereafter by a second dose of about 25 mg per day to about 250 mg per day of Compound I, or a pharmaceutically acceptable salt thereof.

5. The method of claim 3, comprising administering to the subject a first dose of about 200 mg per day to about 900 mg per day of Compound I of formula: 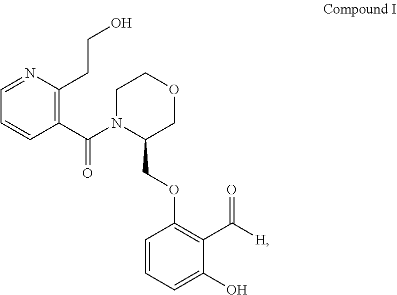
or a pharmaceutically acceptable salt thereof, for one, two, three, four, five, six, or seven days,
followed thereafter by a second dose of about 25 mg per day to about 250 mg per day of Compound I, or a pharmaceutically acceptable salt thereof.

6. The method of any one of claims 1-5, wherein the first dose is administered for one, two, three, or four days.

7. The method of any one of claims 1-6, wherein the first dose is administered for one, two, or three days.

8. The method of any one of claims 1-6, wherein the first dose is administered for four days.

9. The method of any one of claims 1-7, wherein the first dose is administered for three days.

10. The method of any one of claims 1-7, wherein the first dose is administered for two days.

11. The method of any one of claims 1-5, wherein the first dose comprises:
about 600 mg per day of Compound I, or a pharmaceutically acceptable salt thereof, administered for one day;
followed by about 500 mg per day of Compound I, or a pharmaceutically acceptable salt thereof, administered for one day or two days.

12. The method of any one of claims 1-5, wherein the first dose is:
about 700 mg per day of Compound I, or a pharmaceutically acceptable salt thereof, administered for one day;
followed by about 700 mg per day of Compound I, or a pharmaceutically acceptable salt thereof, administered for one day or two days.

13. The method of any one of claims 1-5, wherein the first dose is:
about 800 mg per day of Compound I, or a pharmaceutically acceptable salt thereof, for one day;
followed by about 700 mg per day of Compound I, or a pharmaceutically acceptable salt thereof, administered for one day or two days.

14. The method of any one of claims 1-5, wherein the first dose is:
about 900 mg per day of Compound I, or a pharmaceutically acceptable salt thereof, administered for one day;
followed by about 800 mg per day of Compound I, or a pharmaceutically acceptable salt thereof, administered for one, two, three, or four days.

15. The method of any one of claims 1-5, wherein the first dose is:
about 500 mg per day of Compound I, or a pharmaceutically acceptable salt thereof, administered for one day;
followed by about 400 mg per day of Compound I, or a pharmaceutically acceptable salt thereof, administered for one day or two days.

16. The method of any one of claims 1-4, wherein the first dose is:
about 1000 mg per day of Compound I, or a pharmaceutically acceptable salt thereof, administered for one day;
followed by about 900 mg per day of Compound I, or a pharmaceutically acceptable salt thereof, administered for one day or two days.

17. The method of any one of claims 1-5, wherein the first dose is:
about 300 mg per day of Compound I, or a pharmaceutically acceptable salt thereof, administered for one day;
followed by about 200 mg per day of Compound I, or a pharmaceutically acceptable salt thereof, administered for one day or two days.

18. The method of any one of claims 1-5, wherein the first dose is:
about 600 mg per day of Compound I, or a pharmaceutically acceptable salt thereof, administered for one day;
followed by about 500 mg per day of Compound I, or a pharmaceutically acceptable salt thereof, administered for one day.

19. The method of any one of claims 1-5, wherein the first dose is:
about 700 mg per day of Compound I, or a pharmaceutically acceptable salt thereof, administered for one day;
followed by about 700 mg per day of Compound I, or a pharmaceutically acceptable salt thereof, administered for one day.

20. The method of any one of claims 1-5, wherein the first dose is:
about 800 mg per day of Compound I, or a pharmaceutically acceptable salt thereof, administered for one day;
followed by about 700 mg per day of Compound I, or a pharmaceutically acceptable salt thereof, administered for one day.

21. The method of any one of claims 1-5, wherein the first dose is:
about 900 mg per day of Compound I, or a pharmaceutically acceptable salt thereof, administered for one day;
followed by about 800 mg per day of Compound I, or a pharmaceutically acceptable salt thereof, administered for one, two, or three days.

22. The method of any one of claims 1-5, wherein the first dose is:
about 500 mg per day of Compound I, or a pharmaceutically acceptable salt thereof, administered for one day;
followed by about 400 mg per day of Compound I, or a pharmaceutically acceptable salt thereof, administered for one day.

23. The method of any one of claims 1-4, wherein the first dose is:
about 1000 mg per day of Compound I, or a pharmaceutically acceptable salt thereof, administered for one day;
followed by about 900 mg per day of Compound I, or a pharmaceutically acceptable salt thereof, administered for one day.

24. The method of any one of claims 1-5, wherein the first dose is:
about 300 mg per day of Compound I, or a pharmaceutically acceptable salt thereof, administered for one day;
followed by about 200 mg per day of Compound I, or a pharmaceutically acceptable salt thereof, administered for one day.

25. The method of any one of claims 1-24, wherein the second dose is about 25 mg per day to about 250 mg per day of Compound I, or a pharmaceutically acceptable salt thereof.

26. The method of any one of claims 1-24, wherein the second dose is about 25 mg per day to about 150 mg per day of Compound I, or a pharmaceutically acceptable salt thereof.

27. The method of any one of claims 1-24, wherein the second dose is about 25 mg per day to about 100 mg per day of Compound I, or a pharmaceutically acceptable salt thereof.

28. The method of any one of claims 1-24, wherein the second dose is about 50 mg per day of Compound I, or a pharmaceutically acceptable salt thereof.

29. The method of any one of claims 1-24, wherein the second dose is about 100 mg per day of Compound I, or a pharmaceutically acceptable salt thereof.

30. The method of any one of claims 1-24, wherein the second dose is about 150 mg per day of Compound I, or a pharmaceutically acceptable salt thereof.

31. The method of any one of claims 1-24, wherein the second dose is about 200 mg per day of Compound I, or a pharmaceutically acceptable salt thereof.

32. The method of any one of claims 1-24, wherein the second dose is about 250 mg per day of Compound I, or a pharmaceutically acceptable salt thereof.

33. The method of any one of claims 1-5, wherein:
the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 200 mg per day of Compound I, or a pharmaceutically acceptable salt thereof.

34. The method of any one of claims 1-5, wherein:
the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 175 mg per day of Compound I, or a pharmaceutically acceptable salt thereof.

35. The method of any one of claims 1-5, wherein:
the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 150 mg per day of Compound I, or a pharmaceutically acceptable salt thereof.

36. The method of any one of claims 1-5, wherein:
the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 125 mg per day of Compound I, or a pharmaceutically acceptable salt thereof.

37. The method of any one of claims 1-5, wherein:
the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 100 mg per day of Compound I, or a pharmaceutically acceptable salt thereof.

38. The method of any one of claims 1-5, wherein:
the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 75 mg per day of Compound I, or a pharmaceutically acceptable salt thereof.

39. The method of any one of claims 1-5, wherein:
the first dose is:
about 500 mg per day of Compound I, or a pharmaceutically acceptable salt thereof, administered for one day;
followed by about 400 mg per day of Compound I, or a pharmaceutically acceptable salt thereof, administered for one day; and
followed thereafter by a second dose of about 50 mg per day of Compound I, or a pharmaceutically acceptable salt thereof.

40. The method of any one of claims 1-5, wherein:
the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 200 mg per day of Compound I, or a pharmaceutically acceptable salt thereof.

41. The method of any one of claims 1-5, wherein:
the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 175 mg per day of Compound I, or a pharmaceutically acceptable salt thereof.

42. The method of any one of claims 1-5, wherein:
the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 150 mg per day of Compound I, or a pharmaceutically acceptable salt thereof.

43. The method of any one of claims 1-5 wherein:
the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 125 mg per day of Compound I, or a pharmaceutically acceptable salt thereof.

44. The method of any one of claims 1-5, wherein:
the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 100 mg per day of Compound I, or a pharmaceutically acceptable salt thereof.

45. The method of any one of claims 1-5, wherein:
the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 75 mg per day of Compound I, or a pharmaceutically acceptable salt thereof.

46. The method of any one of claims 1-5, wherein:
the first dose is:
about 300 mg per day of Compound I, or a pharmaceutically acceptable salt thereof, administered for one day;
followed by about 200 mg per day of Compound I, or a pharmaceutically acceptable salt thereof, administered for one day; and
followed thereafter by a second dose of about 50 mg per day of Compound I, or a pharmaceutically acceptable salt thereof.

47. The method of any one of claims 1-5, wherein the first dose is:
about 400 mg per day of Compound I, or a pharmaceutically acceptable salt thereof, administered for four, five, six, or seven days; followed thereafter by a second dose of about 200 mg per day of Compound I, or a pharmaceutically acceptable salt thereof.

48. The method of any one of claims 1-5, wherein the first dose is:
about 400 mg per day of Compound I, or a pharmaceutically acceptable salt thereof, administered for four, five, six, or seven days; followed thereafter by a second dose of about 175 mg per day of Compound I, or a pharmaceutically acceptable salt thereof.

49. The method of any one of claims 1-5, wherein the first dose is:
about 400 mg per day of Compound I, or a pharmaceutically acceptable salt thereof, administered for four, five, six, or seven days; followed thereafter by a second dose of about 150 mg per day of Compound I, or a pharmaceutically acceptable salt thereof.

50. The method of any one of claims 1-5, wherein the first dose is:
about 400 mg per day of Compound I, or a pharmaceutically acceptable salt thereof, administered for four, five, six, or seven days; followed thereafter by a second dose of about 125 mg per day of Compound I, or a pharmaceutically acceptable salt thereof.

51. The method of any one of claims 1-5, wherein the first dose is:
about 400 mg per day of Compound I, or a pharmaceutically acceptable salt thereof, administered for four, five, six, or seven days; followed thereafter by a second dose of about 100 mg per day of Compound I, or a pharmaceutically acceptable salt thereof.

52. The method of any one of claims 1-5, wherein the first dose is:
about 400 mg per day of Compound I, or a pharmaceutically acceptable salt thereof, administered for four, five, six, or seven days; followed thereafter by a second dose of about 75 mg per day of Compound I, or a pharmaceutically acceptable salt thereof.

53. The method of any one of claims 1-5, wherein the first dose is:
about 400 mg per day of Compound I, or a pharmaceutically acceptable salt thereof, administered for four, five, six, or seven days; followed thereafter by a second dose of about 50 mg per day of Compound I, or a pharmaceutically acceptable salt thereof.

54. The method of any one of claims 1-5, wherein the first dose is:
about 600 mg per day of Compound I, or a pharmaceutically acceptable salt thereof, administered for four, five, six, or seven days; followed thereafter by a second dose of about 200 mg per day of Compound I, or a pharmaceutically acceptable salt thereof.

55. The method of any one of claims 1-5, wherein the first dose is:
about 600 mg per day of Compound I, or a pharmaceutically acceptable salt thereof, administered for four, five, six, or seven days, followed thereafter by a second dose of about 175 mg per day of Compound I, or a pharmaceutically acceptable salt thereof.

56. The method of any one of claims 1-5, wherein the first dose is:
about 600 mg per day of Compound I, or a pharmaceutically acceptable salt thereof, administered for four, five, six, or seven days, followed thereafter by a second dose of about 150 mg per day of Compound I, or a pharmaceutically acceptable salt thereof.

57. The method of any one of claims 1-5, wherein the first dose is:
about 600 mg per day of Compound I, or a pharmaceutically acceptable salt thereof, administered for four, five, six, or seven days, followed thereafter by a second dose of about 125 mg per day of Compound I, or a pharmaceutically acceptable salt thereof.

58. The method of any one of claims 1-5, wherein the first dose is:
about 600 mg per day of Compound I, or a pharmaceutically acceptable salt thereof, administered for four, five, six, or seven days, followed thereafter by a second dose of about 100 mg per day of Compound I, or a pharmaceutically acceptable salt thereof.

59. The method of any one of claims 1-5, wherein the first dose is:
about 600 mg per day of Compound I, or a pharmaceutically acceptable salt thereof, administered for four, five, six, or seven days, followed thereafter by a second dose of about 75 mg per day of Compound I, or a pharmaceutically acceptable salt thereof.

60. The method of any one of claims 1-5, wherein the first dose is:
about 600 mg per day of Compound I, or a pharmaceutically acceptable salt thereof, administered for four, five, six, or seven days, followed thereafter by a second dose of about 50 mg per day of Compound I, or a pharmaceutically acceptable salt thereof.

61. The method of any one of claims 1-5, wherein the first dose is:
about 800 mg per day of Compound I, or a pharmaceutically acceptable salt thereof, administered for four, five, six, or seven days, followed thereafter by a second dose of about 200 mg per day of Compound I, or a pharmaceutically acceptable salt thereof.

62. The method of any one of claims 1-5, wherein the first dose is:
about 800 mg per day of Compound I, or a pharmaceutically acceptable salt thereof, administered for four, five, six, or seven days, followed thereafter by a second dose of about 175 mg per day of Compound I, or a pharmaceutically acceptable salt thereof.

63. The method of any one of claims 1-5, wherein the first dose is:
about 800 mg per day of Compound I, or a pharmaceutically acceptable salt thereof, administered for four, five, six, or seven days, followed thereafter by a second dose of about 150 mg per day of Compound I, or a pharmaceutically acceptable salt thereof.

64. The method of any one of claims 1-5, wherein the first dose is:
about 800 mg per day of Compound I, or a pharmaceutically acceptable salt thereof, administered for four, five, six, or seven days, followed thereafter by a second dose of about 125 mg per day of Compound I, or a pharmaceutically acceptable salt thereof.

65. The method of any one of claims 1-5, wherein the first dose is:
about 800 mg per day of Compound I, or a pharmaceutically acceptable salt thereof, administered for four, five, six, or seven days, followed thereafter by a second dose of about 100 mg per day of Compound I, or a pharmaceutically acceptable salt thereof.

66. The method of any one of claims 1-5, wherein the first dose is:
about 800 mg per day of Compound I, or a pharmaceutically acceptable salt thereof, administered for four, five, six, or seven days, followed thereafter by a second dose of about 75 mg per day of Compound I, or a pharmaceutically acceptable salt thereof.

67. The method of any one of claims 1-5, wherein the first dose is:
about 800 mg per day of Compound I, or a pharmaceutically acceptable salt thereof, administered for four, five, six, or seven days, followed thereafter by a second dose of about 50 mg per day of Compound I, or a pharmaceutically acceptable salt thereof.

68. The method of any one of claims 1-67, wherein the first dose of Compound I, or a pharmaceutically acceptable salt thereof, is administered once daily (QD).

69. The method of any one of claims 1-67, wherein the first dose of Compound I, or a pharmaceutically acceptable salt thereof, is administered twice daily (BID).

70. The method of any one of claims 1-67, wherein the first dose of Compound I, or a pharmaceutically acceptable salt thereof, is administered three times daily (TID).

71. The method of any one of claims 1-67, wherein the first dose of Compound I, or a pharmaceutically acceptable salt thereof, is administered four times daily (QID).

72. The method of any one of claims 1-67, wherein the first dose of Compound I, or a pharmaceutically acceptable salt thereof, is administered every six hours (Q4H).

73. The method of any one of claims 1-5, wherein the first dose is:
about 200 mg twice daily (BID) of Compound I, or a pharmaceutically acceptable salt thereof, administered for four, five, six, or seven days; followed thereafter by a second dose of about 200 mg per day of Compound I, or a pharmaceutically acceptable salt thereof.

74. The method of any one of claims 1-5, wherein the first dose is:
about 200 mg twice daily (BID) of Compound I, or a pharmaceutically acceptable salt thereof, administered for four, five, six, or seven days; followed thereafter by a second dose of about 175 mg per day of Compound I, or a pharmaceutically acceptable salt thereof.

75. The method of any one of claims 1-5, wherein the first dose is:
about 200 mg twice daily (BID) of Compound I, or a pharmaceutically acceptable salt thereof, administered for four, five, six, or seven days; followed thereafter by a second dose of about 150 mg per day of Compound I, or a pharmaceutically acceptable salt thereof.

76. The method of any one of claims 1-5, wherein the first dose is:
about 200 mg twice daily (BID) of Compound I, or a pharmaceutically acceptable salt thereof, administered for four, five, six, or seven days; followed thereafter by a second dose of about 125 mg per day of Compound I, or a pharmaceutically acceptable salt thereof.

77. The method of any one of claims 1-5, wherein the first dose is:
about 200 mg twice daily (BID) of Compound I, or a pharmaceutically acceptable salt thereof, administered for four, five, six, or seven days; followed thereafter by a second dose of about 100 mg per day of Compound I, or a pharmaceutically acceptable salt thereof.

78. The method of any one of claims 1-5, wherein the first dose is:
about 200 mg twice daily (BID) of Compound I, or a pharmaceutically acceptable salt thereof, administered for four, five, six, or seven days; followed thereafter by a second dose of about 75 mg per day of Compound I, or a pharmaceutically acceptable salt thereof.

79. The method of any one of claims 1-5, wherein the first dose is:
about 200 mg twice daily (BID) of Compound I, or a pharmaceutically acceptable salt thereof, administered for four, five, six, or seven days; followed thereafter by a second dose of about 50 mg per day of Compound I, or a pharmaceutically acceptable salt thereof.

80. The method of any one of claims 1-5, wherein the first dose is:
about 300 mg twice a day (BID) of Compound I, or a pharmaceutically acceptable salt thereof, administered for four, five, six, or seven days, followed thereafter by a second dose of about 200 mg per day of Compound I, or a pharmaceutically acceptable salt thereof.

81. The method of any one of claims 1-5, wherein the first dose is:
about 300 mg twice a day (BID) of Compound I, or a pharmaceutically acceptable salt thereof, administered for four, five, six, or seven days, followed thereafter by a second dose of about 175 mg per day of Compound I, or a pharmaceutically acceptable salt thereof.

82. The method of any one of claims 1-5, wherein the first dose is:
about 300 mg twice a day (BID) of Compound I, or a pharmaceutically acceptable salt thereof, administered for four, five, six, or seven days, followed thereafter by a second dose of about 150 mg per day of Compound I, or a pharmaceutically acceptable salt thereof.

83. The method of any one of claims 1-5, wherein the first dose is:
about 300 mg twice a day (BID) of Compound I, or a pharmaceutically acceptable salt thereof, administered for four, five, six, or seven days, followed thereafter by a second dose of about 125 mg per day of Compound I, or a pharmaceutically acceptable salt thereof.

84. The method of any one of claims 1-5, wherein the first dose is:
about 300 mg twice a day (BID) of Compound I, or a pharmaceutically acceptable salt thereof, administered for four, five, six, or seven days, followed thereafter by a second dose of about 100 mg per day of Compound I, or a pharmaceutically acceptable salt thereof.

85. The method of any one of claims 1-5, wherein the first dose is:
about 300 mg twice a day (BID) of Compound I, or a pharmaceutically acceptable salt thereof, administered for four, five, six, or seven days, followed thereafter by a second dose of about 75 mg per day of Compound I, or a pharmaceutically acceptable salt thereof.

86. The method of any one of claims 1-5, wherein the first dose is:
about 300 mg twice a day (BID) of Compound I, or a pharmaceutically acceptable salt thereof, administered for four, five, six, or seven days, followed thereafter by a second dose of about 50 mg per day of Compound I, or a pharmaceutically acceptable salt thereof.

87. The method of any one of claims 1-5, wherein the first dose is:
about 200 mg three times daily (TID) of Compound I, or a pharmaceutically acceptable salt thereof, administered for four, five, six, or seven days, followed thereafter by a second dose of about 200 mg per day of Compound I, or a pharmaceutically acceptable salt thereof.

88. The method of any one of claims 1-5, wherein the first dose is:
about 200 mg three times daily (TID) of Compound I, or a pharmaceutically acceptable salt thereof, administered for four, five, six, or seven days, followed thereafter by a second dose of about 175 mg per day of Compound I, or a pharmaceutically acceptable salt thereof.

89. The method of any one of claims 1-5, wherein the first dose is:
about 200 mg three times daily (TID) of Compound I, or a pharmaceutically acceptable salt thereof, administered for four, five, six, or seven days, followed thereafter by a second dose of about 150 mg per day of Compound I, or a pharmaceutically acceptable salt thereof.

90. The method of any one of claims 1-5, wherein the first dose is:
about 200 mg three times daily (TID) of Compound I, or a pharmaceutically acceptable salt thereof, administered for four, five, six, or seven days, followed thereafter by a second dose of about 125 mg per day of Compound I, or a pharmaceutically acceptable salt thereof.

91. The method of any one of claims 1-5, wherein the first dose is:
about 200 mg three times daily (TID) of Compound I, or a pharmaceutically acceptable salt thereof, administered for four, five, six, or seven days, followed thereafter by a second dose of about 100 mg per day of Compound I, or a pharmaceutically acceptable salt thereof.

92. The method of any one of claims 1-5, wherein the first dose is:
about 200 mg three times daily (TID) of Compound I, or a pharmaceutically acceptable salt thereof, administered for four, five, six, or seven days, followed thereafter by a second dose of about 75 mg per day of Compound I, or a pharmaceutically acceptable salt thereof.

93. The method of any one of claims 1-5, wherein the first dose is:
about 200 mg three times daily (TID) of Compound I, or a pharmaceutically acceptable salt thereof, administered for four, five, six, or seven days, followed thereafter by a second dose of about 50 mg per day of Compound I, or a pharmaceutically acceptable salt thereof.

94. The method of any one of claims 1-5, wherein the first dose is:
about 150 mg four times daily (QID) of Compound I, or a pharmaceutically acceptable salt thereof, administered for four, five, six, or seven days, followed thereafter by a second dose of about 200 mg per day of Compound I, or a pharmaceutically acceptable salt thereof.

95. The method of any one of claims 1-5, wherein the first dose is:
about 150 mg four times daily (QID) of Compound I, or a pharmaceutically acceptable salt thereof, administered for four, five, six, or seven days, followed thereafter by a second dose of about 175 mg per day of Compound I, or a pharmaceutically acceptable salt thereof.

96. The method of any one of claims 1-5, wherein the first dose is:
about 150 mg four times daily (QID) of Compound I, or a pharmaceutically acceptable salt thereof, administered for four, five, six, or seven days, followed thereafter by a second dose of about 150 mg per day of Compound I, or a pharmaceutically acceptable salt thereof.

97. The method of any one of claims 1-5, wherein the first dose is:
about 150 mg four times daily (QID) of Compound I, or a pharmaceutically acceptable salt thereof, administered for four, five, six, or seven days, followed thereafter by a second dose of about 125 mg per day of Compound I, or a pharmaceutically acceptable salt thereof.

98. The method of any one of claims 1-5, wherein the first dose is:
about 150 mg four times daily (QID) of Compound I, or a pharmaceutically acceptable salt thereof, administered for four, five, six, or seven days, followed thereafter by a second dose of about 100 mg per day of Compound I, or a pharmaceutically acceptable salt thereof.

99. The method of any one of claims 1-5, wherein the first dose is:
about 150 mg four times daily (QID) of Compound I, or a pharmaceutically acceptable salt thereof, administered for four, five, six, or seven days, followed thereafter by a second dose of about 75 mg per day of Compound I, or a pharmaceutically acceptable salt thereof.

100. The method of any one of claims 1-5, wherein the first dose is:
about 150 mg four times daily (QID) of Compound I, or a pharmaceutically acceptable salt thereof, administered for four, five, six, or seven days, followed thereafter by a second dose of about 50 mg per day of Compound I, or a pharmaceutically acceptable salt thereof.

101. The method of any one of claims 1-5, wherein the first dose is:
about 150 mg every six hours (Q6H) of Compound I, or a pharmaceutically acceptable salt thereof, administered for four, five, six, or seven days, followed thereafter by a second dose of about 200 mg per day of Compound I, or a pharmaceutically acceptable salt thereof.

102. The method of any one of claims 1-5, wherein the first dose is:
about 150 mg every six hours (Q6H) of Compound I, or a pharmaceutically acceptable salt thereof, administered for four, five, six, or seven days, followed thereafter by a second dose of about 175 mg per day of Compound I, or a pharmaceutically acceptable salt thereof.

103. The method of any one of claims 1-5, wherein the first dose is:
about 150 mg every six hours (Q6H) of Compound I, or a pharmaceutically acceptable salt thereof, administered for four, five, six, or seven days, followed thereafter by a second dose of about 150 mg per day of Compound I, or a pharmaceutically acceptable salt thereof.

104. The method of any one of claims 1-5, wherein the first dose is:
about 150 mg every six hours (Q6H) of Compound I, or a pharmaceutically acceptable salt thereof, administered for four, five, six, or seven days, followed thereafter by a second dose of about 125 mg per day of Compound I, or a pharmaceutically acceptable salt thereof.

105. The method of any one of claims 1-5, wherein the first dose is:
about 150 mg every six hours (Q6H) of Compound I, or a pharmaceutically acceptable salt thereof, administered for four, five, six, or seven days, followed thereafter by a second dose of about 100 mg per day of Compound I, or a pharmaceutically acceptable salt thereof.

106. The method of any one of claims 1-5, wherein the first dose is:
about 150 mg every six hours (Q6H) of Compound I, or a pharmaceutically acceptable salt thereof, administered for four, five, six, or seven days, followed thereafter by a second dose of about 75 mg per day of Compound I, or a pharmaceutically acceptable salt thereof.

107. The method of any one of claims 1-5, wherein the first dose is:
about 150 mg every six hours (Q6H) of Compound I, or a pharmaceutically acceptable salt thereof, administered for four, five, six, or seven days, followed thereafter by a second dose of about 50 mg per day of Compound I, or a pharmaceutically acceptable salt thereof.

108. The method of any one of claims 1-5, wherein the first dose is:
about 400 mg twice daily (BID) of Compound I, or a pharmaceutically acceptable salt thereof, administered for four, five, six, or seven days, followed thereafter by a second dose of about 200 mg per day of Compound I, or a pharmaceutically acceptable salt thereof.

109. The method of any one of claims 1-5, wherein the first dose is:
about 400 mg twice daily (BID) of Compound I, or a pharmaceutically acceptable salt thereof, administered for four, five, six, or seven days, followed thereafter by a second dose of about 175 mg per day of Compound I, or a pharmaceutically acceptable salt thereof.

110. The method of any one of claims 1-5, wherein the first dose is:
about 400 mg twice daily (BID) of Compound I, or a pharmaceutically acceptable salt thereof, administered for four, five, six, or seven days, followed thereafter by a second dose of about 150 mg per day of Compound I, or a pharmaceutically acceptable salt thereof.

111. The method of any one of claims 1-5, wherein the first dose is:
about 400 mg twice daily (BID) of Compound I, or a pharmaceutically acceptable salt thereof, administered for four, five, six, or seven days, followed thereafter by a second dose of about 125 mg per day of Compound I, or a pharmaceutically acceptable salt thereof.

112. The method of any one of claims 1-5, wherein the first dose is:
about 400 mg twice daily (BID) of Compound I, or a pharmaceutically acceptable salt thereof, administered for four, five, six, or seven days, followed thereafter by a second dose of about 100 mg per day of Compound I, or a pharmaceutically acceptable salt thereof.

113. The method of any one of claims 1-5, wherein the first dose is:
about 400 mg twice daily (BID) of Compound I, or a pharmaceutically acceptable salt thereof, administered for four, five, six, or seven days, followed thereafter by a second dose of about 75 mg per day of Compound I, or a pharmaceutically acceptable salt thereof.

114. The method of any one of claims 1-5, wherein the first dose is:
about 400 mg twice daily (BID) of Compound I, or a pharmaceutically acceptable salt thereof, administered for four, five, six, or seven days, followed thereafter by a second dose of about 50 mg per day of Compound I, or a pharmaceutically acceptable salt thereof.

115. The method of any one of claims 47-114, wherein the first dose of Compound I, or a pharmaceutically acceptable salt thereof, is administered for four days.

116. The method of any one of claims 47-114, wherein the first dose of Compound I, or a pharmaceutically acceptable salt thereof, is administered for five days.

117. The method of any one of claims 47-114, wherein the first dose of Compound I, or a pharmaceutically acceptable salt thereof, is administered for six days.

118. The method of any one of claims 47-114, wherein the first dose of Compound I, or a pharmaceutically acceptable salt thereof, is administered for seven days.

119. A method for treating sickle cell disease in a subject in need thereof, comprising administering to the subject a first dose of about 200 mg twice daily (BID) of Compound I of formula: 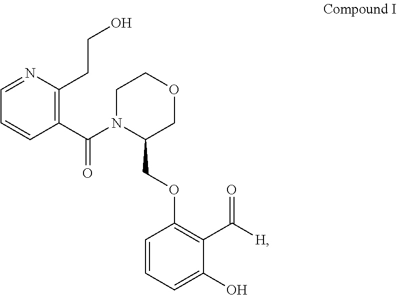
or a pharmaceutically acceptable salt thereof, for four days,
followed thereafter by a second dose of 100 mg per day of Compound I, or a pharmaceutically acceptable salt thereof.

120. A method for treating sickle cell disease in a subject in need thereof, comprising administering to the subject a first dose of about 300 mg twice daily (BID) of Compound I of formula: 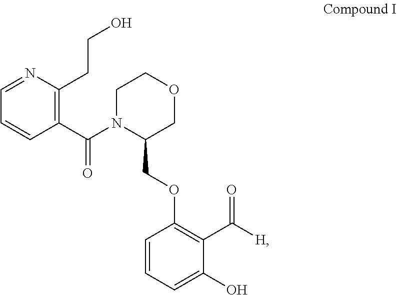
or a pharmaceutically acceptable salt thereof, for four days,
followed thereafter by a second dose of 150 mg per day of Compound I, or a pharmaceutically acceptable salt thereof.

121. A method for treating sickle cell disease in a subject in need thereof, comprising administering to the subject a first dose of about 400 mg twice daily (BID) of Compound I of formula: 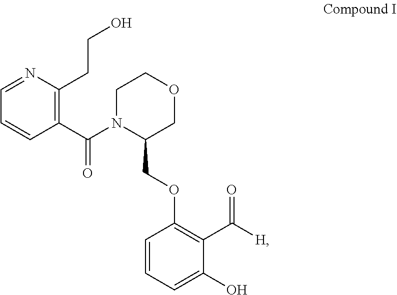
or a pharmaceutically acceptable salt thereof, for four days,
followed thereafter by a second dose of 200 mg per day of Compound I, or a pharmaceutically acceptable salt thereof.

122. The method of any one of claims 1-121, wherein the second dose of Compound I, or a pharmaceutically acceptable salt thereof, is administered for at least one week.

123. The method of any one of claims 1-122, wherein the second dose of Compound I, or a pharmaceutically acceptable salt thereof, is administered for at least ten weeks.

124. The method of any one of claims 1-123, wherein the second dose of Compound I, or a pharmaceutically acceptable salt thereof, is administered for at least fifty weeks.

125. The method of any one of claims 1-124, wherein the second dose of Compound I, or a pharmaceutically acceptable salt thereof, is administered for at least one year.

126. The method of any one of claims 1-125, wherein the second dose of Compound I, or a pharmaceutically acceptable salt thereof, is administered for at least five years.

127. The method of any one of claims 1-126, wherein the second dose of Compound I, or a pharmaceutically acceptable salt thereof, is administered for the lifetime of the subject.

128. The method of any one of claims 1-127, wherein the second dose of Compound I, or a pharmaceutically acceptable salt thereof, is administered once daily (QD).

129. The method of any one of claims 1-128, wherein Compound I, or a pharmaceutically acceptable salt thereof, is administered as a pharmaceutical composition.

130. The method of any one of claims 1-129, wherein Compound I is administered as a pharmaceutical composition.

131. The method of any one of claims 1-130, wherein Compound I, or a pharmaceutically acceptable salt thereof, is administered orally.

132. The method of any one of claims 1-131, wherein Compound I is a crystalline form characterized by an X-ray powder diffractogram comprising the following peaks: 18.3°, 23.4°, and 26.1 °2θ±0.2 °2θ (Compound I Form I), as determined on a diffractometer using Cu-Kα radiation.

133. The method of claim 132, wherein the diffractogram further comprises one or more peaks at:
10.8° or 17.3 °2θ±0.2 °2θ.

134. The method of claim 132 or 133, wherein the crystalline form is characterized by a differential scanning calorimetry (DSC) curve comprising an endotherm at about 111° C. (onset temperature).

135. The method of any one of claims 1-134, further comprising administering an additional therapeutic agent.

136. The method of claim 135, wherein the additional therapeutic agent is hydroxyurea.

137. The method of any one of claims 1-136, wherein the percent hemoglobin occupancy in the subject, as provide by the administration of Compound I, or a pharmaceutically acceptable salt thereof, reaches about 25% to about 60% after about 8 hours to about 24 hours following the completion of the first dose (or loading dose) of Compound I, or a pharmaceutically acceptable salt thereof.

138. The method of claim 137, wherein the percent hemoglobin occupancy in the subject, as provided by the administration of Compound I, or a pharmaceutically acceptable salt thereof, reaches about 25% to about 60% after about 8 hours to about 12 hours following the completion of the first dose (or loading dose) of Compound I, or a pharmaceutically acceptable salt thereof.

139. The method of any one of claims 1-136, wherein the percent hemoglobin occupancy in the subject, as provided by the administration of Compound I, or a pharmaceutically acceptable salt thereof, reaches about 25% to about 60% prior to administration of the second dose (or maintenance dose) of Compound I, or a pharmaceutically acceptable salt thereof.

140. The method of any one of claims 1-139, wherein the administration of Compound I, or a pharmaceutically acceptable salt thereof, provides a blood concentration of Compound I of about 50 μg/mL to about 800 μg/mL.

141. The method of claim 137, wherein the administration of Compound I, or a pharmaceutically acceptable salt thereof, provides a blood concentration of Compound I of about 300 μg/mL to about 475 μg/mL.

142. A compound for use in a method of treating sickle cell disease in a subject in need thereof, wherein the compound is Compound I of formula: 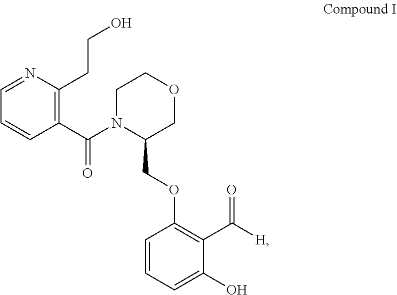
or a pharmaceutically acceptable salt thereof, and wherein the method is the method of any one of claims 1-141.

143. A compound for use in a method of treating sickle cell disease in a subject in need thereof, wherein the compound is Compound I of formula: 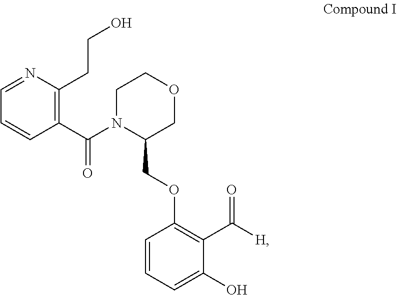
or a pharmaceutically acceptable salt thereof, and wherein the method comprises administering to the subject a first dose of Compound I, or a pharmaceutically acceptable salt thereof, for one, two, three, four, five, six, or seven days, followed thereafter by a second dose of Compound I, or a pharmaceutically acceptable salt thereof, wherein the administrations of the first dose and the second dose provide about 25% to about 60% hemoglobin occupancy by Compound I.

144. A compound for use in a method of treating sickle cell disease in a subject in need thereof, wherein the compound is Compound I of formula: 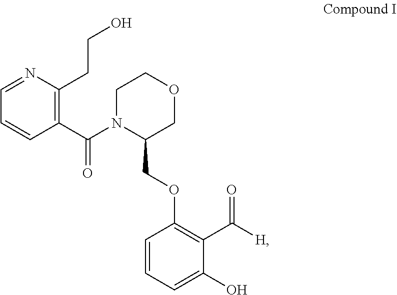
or a pharmaceutically acceptable salt thereof, and wherein the method comprises administering to the subject a first dose of Compound I, or a pharmaceutically acceptable salt thereof, for one, two, three, four, five, six, or seven days, followed thereafter by a second dose of about 25 mg per day to about 500 mg per day of Compound I, or a pharmaceutically acceptable salt thereof.

145. A compound for use in a method of treating sickle cell disease in a subject in need thereof, wherein the compound is Compound I of formula: 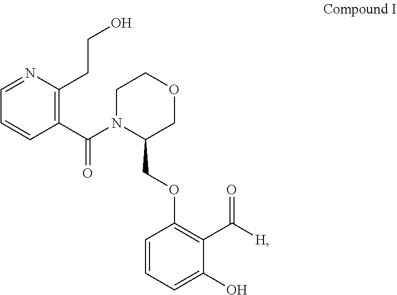
or a pharmaceutically acceptable salt thereof, and wherein the method comprises administering to the subject a first dose of about 200 mg twice daily (BID) of Compound I, or a pharmaceutically acceptable salt thereof, for four days,
followed thereafter by a second dose of 100 mg per day of Compound I, or a pharmaceutically acceptable salt thereof.

146. A compound for use in a method of treating sickle cell disease in a subject in need thereof, wherein the compound is Compound I of formula: 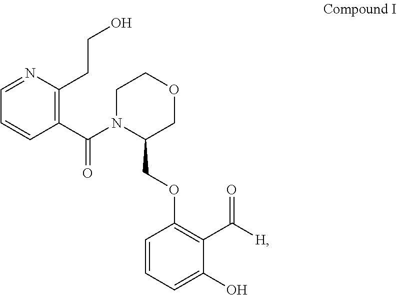
or a pharmaceutically acceptable salt thereof, and wherein the method comprises administering to the subject a first dose of about 300 mg twice daily (BID) of Compound I, or a pharmaceutically acceptable salt thereof, for four days,
followed thereafter by a second dose of 150 mg per day of Compound I, or a pharmaceutically acceptable salt thereof.

147. A compound for use in a method of treating sickle cell disease in a subject in need thereof, wherein the compound is Compound I of formula: 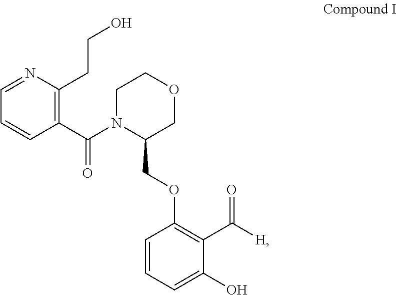
or a pharmaceutically acceptable salt thereof, and wherein the method comprises administering to the subject a first dose of about 400 mg twice daily (BID) of Compound I, or a pharmaceutically acceptable salt thereof, for four days,
followed thereafter by a second dose of 200 mg per day of Compound I, or a pharmaceutically acceptable salt thereof.

148. A compound for use in a method of treating, as claimed in claim 143, wherein the administration of the first dose and the second dose of Compound I provide about 30% to about 50% hemoglobin occupancy by Compound I.

149. A compound for use in a method of treating, as claimed in any one of claims 143, 144, and 148, wherein the method comprises administering to the subject a first dose of about 300 mg per day to about 1500 mg per day of Compound I, or a pharmaceutically acceptable salt thereof, for one, two, three, four, five, six, or seven days, followed thereafter by a second dose of about 25 mg per day to about 250 mg per day of Compound I, or a pharmaceutically acceptable salt thereof.

150. A compound for use in a method of treating, as claimed in any one of claims 143, 144, and 148, wherein the method comprises administering to the subject a first dose of about 200 mg per day to about 900 mg per day of Compound I, or a pharmaceutically acceptable salt thereof, for one, two, three, four, five, six, or seven days, followed thereafter by a second dose of about 25 mg per day to about 250 mg per day of Compound I, or a pharmaceutically acceptable salt thereof.

151. A compound for use in a method of treating, as claimed in any one of claims 143, 144, and 148-150, wherein the second dose is about 25 mg per day, 50 mg per day, 100 mg per day, 125 mg per day, 150 mg per day, 200 mg per day, or 250 mg per day of Compound I, or a pharmaceutically acceptable salt thereof.

152. A compound for use in a method of treating, as claimed in any one of claims 143, 144, and 148-151, wherein the first dose is about 400 mg per day of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

153. A compound for use in a method of treating, as claimed in any one of claims 143, 144, and 148-151, wherein the first dose is about 600 mg per day of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

154. A compound for use in a method of treating, as claimed in any one of claims 143, 144, and 148-151, wherein the first dose is about 800 mg per day of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

155. A compound for use in a method of treating, as claimed in any one of claims 143, 144, and 148-154, wherein the first dose of Compound I, or a pharmaceutically acceptable salt thereof, is administered once daily (QD), twice daily (BID), three times daily (TID), four times daily (QID), or every six hours (Q4H).

156. A compound for use in a method of treating, as claimed in any one of claims 143, 144, and 148-151, wherein the first dose is about 200 mg twice daily (BID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

157. A compound for use in a method of treating, as claimed in any one of claims 143, 144, and 148-151, wherein the first dose is about 300 mg twice daily (BID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

158. A compound for use in a method of treating, as claimed in any one of claims 143, 144, and 148-151, wherein the first dose is about 200 mg three times daily (TID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

159. A compound for use in a method of treating, as claimed in any one of claims 143, 144, and 148-151, wherein the first dose is about 150 mg four times daily (QID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

160. A compound for use in a method of treating, as claimed in any one of claims 143, 144, and 148-151, wherein the first dose is about 150 mg every six hours (Q6H) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

161. A compound for use in a method of treating, as claimed in any one of claims 143, 144, and 148-151, wherein the first dose is about 400 mg twice daily (BID) of Compound I, or a pharmaceutically acceptable salt thereof, administered for four, five, six, or seven days, followed thereafter by a second dose of about 25 mg per day, 50 mg per day, 75 mg per day, 100 mg per day, 125 mg per day, 150 mg per day, 175 mg per day, or 200 mg per day of Compound I, or a pharmaceutically acceptable salt thereof.

162. A compound for use in a method of treating, as claimed in any one of claims 143-161, wherein the first dose of Compound I, or a pharmaceutically acceptable salt thereof, is administered for four days.

163. A compound for use in a method of treating, as claimed in any one of claims 143-162, wherein the second dose of Compound I, or a pharmaceutically acceptable salt thereof, is administered for at least one week, at least ten weeks, or at least fifty weeks.

164. A compound for use in a method of treating, as claimed in any one of claims 143-163, wherein the second dose of Compound I, or a pharmaceutically acceptable salt thereof, is administered for at least one year, at least five years, or at least ten years.

165. A compound for use in a method of treating, as claimed in any one of claims 143-164, wherein the second dose of Compound I, or a pharmaceutically acceptable salt thereof, is administered for the lifetime of the subject.

166. A compound for use in a method of treating, as claimed in any one of claims 143-165, wherein the second dose of Compound I, or a pharmaceutically acceptable salt thereof, is administered once daily (QD).

167. A compound for use in a method of treating, as claimed in any one of claims 143-166, wherein the second dose of Compound I, or a pharmaceutically acceptable salt thereof, is administered as a pharmaceutical composition.

168. A compound for use in a method of treating, as claimed in any one of claims 143-167, wherein Compound I, or a pharmaceutically acceptable salt thereof, is administered orally.

169. A compound for use in a method of treating, as claimed in any one of claims 143-168, wherein Compound I is a crystalline form characterized by an X-ray powder diffractogram comprising the following peaks: 18.3°, 23.4°, and 26.1 °2θ±0.2 °2θ (Compound I Form I), as determined on a diffractometer using Cu-Kα radiation.

170. A compound for use in a method of treating, as claimed in claim 169, wherein the diffractogram further comprises one or more peaks at: 10.8° or 17.3 °2θ±0.2 °2θ.

171. A compound for use in a method of treating, as claimed in claim 169 or 170, wherein the crystalline form is characterized by a differential scanning calorimetry (DSC) curve comprising an endotherm at about 111° C. (onset temperature).

172. A compound for use in a method of treating, as claimed in any one of claims 143-171, further comprising administering an additional therapeutic agent.

173. A compound for use in a method of treating, as claimed in claim 172, wherein the additional therapeutic agent is hydroxyurea.

174. A compound for use in a method of treating, as claimed in any one of claims 143-173, wherein the percent hemoglobin occupancy in the subject, as provided by the administration of Compound I, or a pharmaceutically acceptable salt thereof, reaches about 25% to about 60% after about 8 hours to about 24 hours following the completion of the first dose (or loading dose) of Compound I, or a pharmaceutically acceptable salt thereof.

175. A compound for use in a method of treating, as claimed in any one of claims 143-174, wherein the percent hemoglobin occupancy in the subject, as provided by the administration of Compound I, or a pharmaceutically acceptable salt thereof, reaches about 25% to about 60% after about 8 hours to about 12 hours following the completion of the first dose (or loading dose) of Compound I, or a pharmaceutically acceptable salt thereof.

176. A compound for use in a method of treating, as claimed in any one of claims 143-175, wherein the percent hemoglobin occupancy in the subject, as provided by the administration of Compound I, or a pharmaceutically acceptable salt thereof, reaches about 25% to about 60% prior to administration of the second dose (or maintenance dose) of Compound I, or a pharmaceutically acceptable salt thereof.

177. A compound for use in a method of treating, as claimed in any one of claims 143-176, wherein the administration of Compound I, or a pharmaceutically acceptable salt thereof, provides a blood concentration of Compound I of about 50 μg/mL to about 800 μg/mL.

178. A compound for use in a method of treating, as claimed in claim 177, wherein the administration of Compound I, or a pharmaceutically acceptable salt thereof, provides a blood concentration of Compound I of about 300 μg/mL to about 475 μg/mL.
